SEQUENCE LISTING (201 sequences)

SEQ ID NO: 1 - RNA, 21 nt, synthetic construct
  source 1..21: mol_type = other RNA
gctcttcagtttcacaccatt

SEQ ID NO: 2 - RNA, 21 nt, synthetic construct
  source 1..21: mol_type = other RNA
tggtgtgaaactgaagagctt

SEQ ID NO: 3 - RNA, 19 nt, synthetic construct
  source 1..19: mol_type = other RNA
ggttcgattttcatttgag

SEQ ID NO: 4 - RNA, 19 nt, synthetic construct
  source 1..19: mol_type = other RNA
ctcaaatgaaaatcgaacc

SEQ ID NO: 5 - RNA, 24 nt, synthetic construct
  source 1..24: mol_type = other RNA
agatgacacgattctttaccaaat

SEQ ID NO: 6 - RNA, 25 nt, synthetic construct
  source 1..25: mol_type = other RNA
agattgtagatatgaagcgtgccgt

SEQ ID NO: 7 - RNA, 24 nt, synthetic construct
  source 1..24: mol_type = other RNA
ggaatagtacttacgcaatgctcg

SEQ ID NO: 8 - RNA, 19 nt, synthetic construct
  source 1..19: mol_type = other RNA
ccgtaactctagtacagct

SEQ ID NO: 9 - RNA, 19 nt, synthetic construct
  source 1..19: mol_type = other RNA
agctgtactagagttacgg

SEQ ID NO: 10 - RNA, 25 nt, synthetic construct
  source 1..25: mol_type = other RNA
agcggacactcaatgagatctacca

SEQ ID NO: 11 - RNA, 25 nt, synthetic construct
  source 1..25: mol_type = other RNA
tcaagatcaaccaaaccaccatgga

SEQ ID NO: 12 - RNA, 25 nt, synthetic construct
  source 1..25: mol_type = other RNA
gccaaacagagccttcacaaccagc

SEQ ID NO: 13 - RNA, 25 nt, synthetic construct
  source 1..25: mol_type = other RNA
acaccaaacctgctcactaaaaaaa

SEQ ID NO: 14 - RNA, 25 nt, synthetic construct
  source 1..25: mol_type = other RNA
gaattttcatttcaaccagtttgca

SEQ ID NO: 15 - RNA, 25 nt, synthetic construct
  source 1..25: mol_type = other RNA
ataaactcaacgttgaaaatgtcct

SEQ ID NO: 16 - RNA, 19 nt, synthetic construct
  source 1..19: mol_type = other RNA
gacattacatccacttata

SEQ ID NO: 17 - RNA, 19 nt, synthetic construct
  source 1..19: mol_type = other RNA
ttaaaggacttgtaccata

SEQ ID NO: 18 - RNA, 19 nt, synthetic construct
  source 1..19: mol_type = other RNA
gcacatcacatctgtaaca

SEQ ID NO: 19 - RNA, 19 nt, synthetic construct
  source 1..19: mol_type = other RNA
gtacacagcaggacgaaga

SEQ ID NO: 20 - RNA, 29 nt, synthetic construct
  source 1..29: mol_type = other RNA
gtcaagagaagctggcctgattatgtgga

SEQ ID NO: 21 - RNA, 29 nt, synthetic construct
  source 1..29: mol_type = other RNA
cctgtggctactgaggtagttgacaaaga

SEQ ID NO: 22 - RNA, 29 nt, synthetic construct
  source 1..29: mol_type = other RNA
gaaggagttccagcttctgctcgccaata

SEQ ID NO: 23 - RNA, 29 nt, synthetic construct
  source 1..29: mol_type = other RNA
cagaacctcattccttctttggagcaggc

SEQ ID NO: 24 - RNA, 25 nt, synthetic construct
  source 1..25: mol_type = other RNA
gtacctttcgagaatctctatttgt

SEQ ID NO: 25 - RNA, 25 nt, synthetic construct
  source 1..25: mol_type = other RNA
ctgaagcacctgttgtgcaatctga

SEQ ID NO: 26 - RNA, 25 nt, synthetic construct
  source 1..25: mol_type = other RNA
agcatcgggtgttgttgtcatcaca

SEQ ID NO: 27 - RNA, 19 nt, synthetic construct
  source 1..19: mol_type = other RNA
gctctgacgcatacggatt

SEQ ID NO: 28 - RNA, 19 nt, synthetic construct
  source 1..19: mol_type = other RNA
ctgaaatgcttctgcgtaa

SEQ ID NO: 29 - RNA, 19 nt, synthetic construct
  source 1..19: mol_type = other RNA
acaacaagaagctgacgta

SEQ ID NO: 30 - RNA, 19 nt, synthetic construct
  source 1..19: mol_type = other RNA
cctatcaagcacacgcaaa

SEQ ID NO: 31 - RNA, 22 nt, synthetic construct
  source 1..22: mol_type = other RNA
ggagaagcgtcagatagcgcgt

SEQ ID NO: 32 - RNA, 25 nt, synthetic construct
  source 1..25: mol_type = other RNA
agaactacacgcagtacatccctct

SEQ ID NO: 33 - RNA, 25 nt, synthetic construct
  source 1..25: mol_type = other RNA
agattggacgaccaaatatttctga

SEQ ID NO: 34 - RNA, 19 nt, synthetic construct
  source 1..19: mol_type = other RNA
agaaccaggttgcaagatt

SEQ ID NO: 35 - RNA, 19 nt, synthetic construct
  source 1..19: mol_type = other RNA
aatcttgcaacctggttct

SEQ ID NO: 36 - DNA, 20 bp, synthetic construct
  source 1..20: mol_type = other DNA
agcggcggcaagaaataatg

SEQ ID NO: 37 - DNA, 20 bp, synthetic construct
  source 1..20: mol_type = other DNA
aacccaacatccaaggcact

SEQ ID NO: 38 - DNA, 21 bp, synthetic construct
  source 1..21: mol_type = other DNA
gggtagcagcagatgtagttt

SEQ ID NO: 39 - DNA, 21 bp, synthetic construct
  source 1..21: mol_type = other DNA
ccagtcacaaacagtcctaca

SEQ ID NO: 40 - DNA, 19 bp, synthetic construct
  source 1..19: mol_type = other DNA
cctggtgtggtgtggttta

SEQ ID NO: 41 - DNA, 22 bp, synthetic construct
  source 1..22: mol_type = other DNA
atccttctctatgtgggctttc

SEQ ID NO: 42 - DNA, 22 bp, synthetic construct
  source 1..22: mol_type = other DNA
ggtgcattacagagaggagaaa

SEQ ID NO: 43 - DNA, 22 bp, synthetic construct
  source 1..22: mol_type = other DNA
gtgtgtttcacgcacagataag

SEQ ID NO: 44 - DNA, 22 bp, synthetic construct
  source 1..22: mol_type = other DNA
tctatggtcacaggagagaagg

SEQ ID NO: 45 - DNA, 23 bp, synthetic construct
  source 1..23: mol_type = other DNA
catgcctatgtctggctctattc

SEQ ID NO: 46 - DNA, 22 bp, synthetic construct
  source 1..22: mol_type = other DNA
cctgatgtacctgatgctatgg

SEQ ID NO: 47 - DNA, 21 bp, synthetic construct
  source 1..21: mol_type = other DNA
cctcctgaaacatcgcactat

SEQ ID NO: 48 - DNA, 20 bp, synthetic construct
  source 1..20: mol_type = other DNA
gaaccagcagagtggagatg

SEQ ID NO: 49 - DNA, 22 bp, synthetic construct
  source 1..22: mol_type = other DNA
ggcatagatacctgggctaatg

SEQ ID NO: 50 - DNA, 23 bp, synthetic construct
  source 1..23: mol_type = other DNA
gacaatggcttactccaggagac

SEQ ID NO: 51 - DNA, 21 bp, synthetic construct
  source 1..21: mol_type = other DNA
tgctgccttcttctgtccatg

SEQ ID NO: 52 - DNA, 20 bp, synthetic construct
  source 1..20: mol_type = other DNA
cagagctcctacccactgct

SEQ ID NO: 53 - DNA, 20 bp, synthetic construct
  source 1..20: mol_type = other DNA
cttctccttctccagcacca

SEQ ID NO: 54 - DNA, 19 bp, synthetic construct
  source 1..19: mol_type = other DNA
ccaggacagaccacattca

SEQ ID NO: 55 - DNA, 20 bp, synthetic construct
  source 1..20: mol_type = other DNA
ctggacacccattccagact

SEQ ID NO: 56 - DNA, 20 bp, synthetic construct
  source 1..20: mol_type = other DNA
tgcaccaccaactgcttagc

SEQ ID NO: 57 - DNA, 21 bp, synthetic construct
  source 1..21: mol_type = other DNA
ggcatggactgtggtcatgag

SEQ ID NO: 58 - DNA, 18 bp, synthetic construct
  source 1..18: mol_type = other DNA
caccaccaactgcttagc

SEQ ID NO: 59 - DNA, 21 bp, synthetic construct
  source 1..21: mol_type = other DNA
ggcatggactgtggtcatgag

SEQ ID NO: 60 - DNA, 20 bp, synthetic construct
  source 1..20: mol_type = other DNA
aggcagggagtgtgtgaact

SEQ ID NO: 61 - DNA, 20 bp, synthetic construct
  source 1..20: mol_type = other DNA
gtggttgtggtggtctgaca

SEQ ID NO: 62 - DNA, 20 bp, synthetic construct
  source 1..20: mol_type = other DNA
cttgggcctcgacttacatc

SEQ ID NO: 63 - DNA, 20 bp, synthetic construct
  source 1..20: mol_type = other DNA
cttgggcctcgacttacatc

SEQ ID NO: 64 - DNA, 20 bp, synthetic construct
  source 1..20: mol_type = other DNA
catgtgtgtccagcctgaat

SEQ ID NO: 65 - DNA, 20 bp, synthetic construct
  source 1..20: mol_type = other DNA
ggcacagtcgatacactgga

SEQ ID NO: 66 - DNA, 22 bp, synthetic construct
  source 1..22: mol_type = other DNA
gtaacccagggaggtcaataaa

SEQ ID NO: 67 - DNA, 23 bp, synthetic construct
  source 1..23: mol_type = other DNA
gcctctacatcactagcactaac

SEQ ID NO: 68 - DNA, 20 bp, synthetic construct
  source 1..20: mol_type = other DNA
gagaaagcgggtctcggaag

SEQ ID NO: 69 - DNA, 20 bp, synthetic construct
  source 1..20: mol_type = other DNA
attggccattgcacgagtga

SEQ ID NO: 70 - DNA, 22 bp, synthetic construct
  source 1..22: mol_type = other DNA
gtaacccagggaggtcaataaa

SEQ ID NO: 71 - DNA, 23 bp, synthetic construct
  source 1..23: mol_type = other DNA
gcctctacatcactagcactaac

SEQ ID NO: 72 - DNA, 20 bp, synthetic construct
  source 1..20: mol_type = other DNA
tgatcgattgggctgcttta

SEQ ID NO: 73 - DNA, 22 bp, synthetic construct
  source 1..22: mol_type = other DNA
cagcacgagtagagggatattg

SEQ ID NO: 74 - DNA, 21 bp, synthetic construct
  source 1..21: mol_type = other DNA
tgaagagtgtgacaggaaacc

SEQ ID NO: 75 - DNA, 20 bp, synthetic construct
  source 1..20: mol_type = other DNA
cacctcgctgctctcaatta

SEQ ID NO: 76 - DNA, 21 bp, synthetic construct
  source 1..21: mol_type = other DNA
gaggaaggtgtgtggagattc

SEQ ID NO: 77 - DNA, 22 bp, synthetic construct
  source 1..22: mol_type = other DNA
gcctaggtggttgagagtaatg

SEQ ID NO: 78 - DNA, 20 bp, synthetic construct
  source 1..20: mol_type = other DNA
agaacagctgcacccacttc

SEQ ID NO: 79 - DNA, 20 bp, synthetic construct
  source 1..20: mol_type = other DNA
gcatcacctcctccaggtaa

SEQ ID NO: 80 - DNA, 20 bp, synthetic construct
  source 1..20: mol_type = other DNA
atattggggcttgcctttct

SEQ ID NO: 81 - DNA, 20 bp, synthetic construct
  source 1..20: mol_type = other DNA
gtgtaattccagggggaggt

SEQ ID NO: 82 - DNA, 20 bp, synthetic construct
  source 1..20: mol_type = other DNA
gcattcatgaccaggatcac

SEQ ID NO: 83 - DNA, 21 bp, synthetic construct
  source 1..21: mol_type = other DNA
caaagaagatctctgggcgta

SEQ ID NO: 84 - DNA, 22 bp, synthetic construct
  source 1..22: mol_type = other DNA
ctttgaggaattggtcgagagg

SEQ ID NO: 85 - DNA, 21 bp, synthetic construct
  source 1..21: mol_type = other DNA
gagagccataaggtgctgtca

SEQ ID NO: 86 - DNA, 20 bp, synthetic construct
  source 1..20: mol_type = other DNA
tccgacagtggttgatcgac

SEQ ID NO: 87 - DNA, 22 bp, synthetic construct
  source 1..22: mol_type = other DNA
cctcacgattgtagtcctgctt

SEQ ID NO: 88 - DNA, 20 bp, synthetic construct
  source 1..20: mol_type = other DNA
caccctgaccttgcctattt

SEQ ID NO: 89 - DNA, 18 bp, synthetic construct
  source 1..18: mol_type = other DNA
agctgcttggcggattag

SEQ ID NO: 90 - DNA, 21 bp, synthetic construct
  source 1..21: mol_type = other DNA
tgggatcacctttgacctttc

SEQ ID NO: 91 - DNA, 21 bp, synthetic construct
  source 1..21: mol_type = other DNA
ggctcggctattgtcctttat

SEQ ID NO: 92 - DNA, 21 bp, synthetic construct
  source 1..21: mol_type = other DNA
ctgaaggacgaaggactgaaa

SEQ ID NO: 93 - DNA, 19 bp, synthetic construct
  source 1..19: mol_type = other DNA
agccagcagcagtgataaa

SEQ ID NO: 94 - DNA, 20 bp, synthetic construct
  source 1..20: mol_type = other DNA
gttcccatcggagcgatcat

SEQ ID NO: 95 - DNA, 20 bp, synthetic construct
  source 1..20: mol_type = other DNA
gtgctgctgaggaatccagt

SEQ ID NO: 96 - DNA, 20 bp, synthetic construct
  source 1..20: mol_type = other DNA
gttcccatcggagcgatcat

SEQ ID NO: 97 - DNA, 20 bp, synthetic construct
  source 1..20: mol_type = other DNA
gtgctgctgaggaatccagt

SEQ ID NO: 98 - DNA, 20 bp, synthetic construct
  source 1..20: mol_type = other DNA
ggaagtggtgctggttagga

SEQ ID NO: 99 - DNA, 20 bp, synthetic construct
  source 1..20: mol_type = other DNA
tgatgagaacaagggctgtg

SEQ ID NO: 100 - DNA, 20 bp, synthetic construct
  source 1..20: mol_type = other DNA
gaaagcaggagcaatggaag

SEQ ID NO: 101 - DNA, 20 bp, synthetic construct
  source 1..20: mol_type = other DNA
agagggcaatctcatcctca

SEQ ID NO: 102 - DNA, 20 bp, synthetic construct
  source 1..20: mol_type = other DNA
aaggacaacggccacaagta

SEQ ID NO: 103 - DNA, 20 bp, synthetic construct
  source 1..20: mol_type = other DNA
acgatcacggcgagaatgaa

SEQ ID NO: 104 - DNA, 20 bp, synthetic construct
  source 1..20: mol_type = other DNA
ctgcccatggctttcaaggt

SEQ ID NO: 105 - DNA, 23 bp, synthetic construct
  source 1..23: mol_type = other DNA
gccgagtagttttcatcattgcc

SEQ ID NO: 106 - DNA, 22 bp, synthetic construct
  source 1..22: mol_type = other DNA
ccttcccaagtacaccatcatc

SEQ ID NO: 107 - DNA, 21 bp, synthetic construct
  source 1..21: mol_type = other DNA
ctgccacatcgacctctaaac

SEQ ID NO: 108 - DNA, 22 bp, synthetic construct
  source 1..22: mol_type = other DNA
caggtcgttctgatagtgaagg

SEQ ID NO: 109 - DNA, 22 bp, synthetic construct
  source 1..22: mol_type = other DNA
cttgtgggactgatttcgtttg

SEQ ID NO: 110 - DNA, 20 bp, synthetic construct
  source 1..20: mol_type = other DNA
ggagaccatggagaccctca

SEQ ID NO: 111 - DNA, 21 bp, synthetic construct
  source 1..21: mol_type = other DNA
gtcagggaactctcccacttg

SEQ ID NO: 112 - DNA, 21 bp, synthetic construct
  source 1..21: mol_type = other DNA
agagaaatgagtgagtgtggg

SEQ ID NO: 113 - DNA, 21 bp, synthetic construct
  source 1..21: mol_type = other DNA
acaggaggtgttcccctttgc

SEQ ID NO: 114 - DNA, 20 bp, synthetic construct
  source 1..20: mol_type = other DNA
ccagggaaccgcttatatgt

SEQ ID NO: 115 - DNA, 20 bp, synthetic construct
  source 1..20: mol_type = other DNA
ctgggtcacattgttggaag

SEQ ID NO: 116 - DNA, 20 bp, synthetic construct
  source 1..20: mol_type = other DNA
cacctggagctgtaccagaa

SEQ ID NO: 117 - DNA, 20 bp, synthetic construct
  source 1..20: mol_type = other DNA
tgcagtgtgttatccctgct

SEQ ID NO: 118 - DNA, 20 bp, synthetic construct
  source 1..20: mol_type = other DNA
ccatcaccggcagaacacag

SEQ ID NO: 119 - DNA, 20 bp, synthetic construct
  source 1..20: mol_type = other DNA
atccagctgcagagcattga

SEQ ID NO: 120 - DNA, 20 bp, synthetic construct
  source 1..20: mol_type = other DNA
gcggcggcaagaaataatgg

SEQ ID NO: 121 - DNA, 20 bp, synthetic construct
  source 1..20: mol_type = other DNA
aacccaacatccaaggcact

SEQ ID NO: 122 - DNA, 20 bp, synthetic construct
  source 1..20: mol_type = other DNA
cacaaagtgctgctgttctg

SEQ ID NO: 123 - DNA, 22 bp, synthetic construct
  source 1..22: mol_type = other DNA
ttccatcaagcctgaggtattt

SEQ ID NO: 124 - DNA, 22 bp, synthetic construct
  source 1..22: mol_type = other DNA
ggataagacccgagctttgatt

SEQ ID NO: 125 - DNA, 22 bp, synthetic construct
  source 1..22: mol_type = other DNA
ctgccaggtaactgtcttcttc

SEQ ID NO: 126 - DNA, 22 bp, synthetic construct
  source 1..22: mol_type = other DNA
ggtgcattacagagaggagaaa

SEQ ID NO: 127 - DNA, 22 bp, synthetic construct
  source 1..22: mol_type = other DNA
gtgtgtttcacgcacagataag

SEQ ID NO: 128 - DNA, 22 bp, synthetic construct
  source 1..22: mol_type = other DNA
ggactcaagtccttacctcttc

SEQ ID NO: 129 - DNA, 20 bp, synthetic construct
  source 1..20: mol_type = other DNA
cctggctcaacatgctacta

SEQ ID NO: 130 - DNA, 21 bp, synthetic construct
  source 1..21: mol_type = other DNA
ggttgacttccggctttctaa

SEQ ID NO: 131 - DNA, 22 bp, synthetic construct
  source 1..22: mol_type = other DNA
tcttctggctgctcacaatatc

SEQ ID NO: 132 - DNA, 22 bp, synthetic construct
  source 1..22: mol_type = other DNA
tctatggtcacaggagagaagg

SEQ ID NO: 133 - DNA, 23 bp, synthetic construct
  source 1..23: mol_type = other DNA
catgcctatgtctggctctattc

SEQ ID NO: 134 - DNA, 22 bp, synthetic construct
  source 1..22: mol_type = other DNA
cctgatgtacctgatgctatgg

SEQ ID NO: 135 - DNA, 21 bp, synthetic construct
  source 1..21: mol_type = other DNA
cctcctgaaacatcgcactat

SEQ ID NO: 136 - DNA, 23 bp, synthetic construct
  source 1..23: mol_type = other DNA
cacagagagacagacttgagaac

SEQ ID NO: 137 - DNA, 21 bp, synthetic construct
  source 1..21: mol_type = other DNA
acttggatacccttggcttta

SEQ ID NO: 138 - DNA, 20 bp, synthetic construct
  source 1..20: mol_type = other DNA
ggccatctgcgtatcttctt

SEQ ID NO: 139 - DNA, 21 bp, synthetic construct
  source 1..21: mol_type = other DNA
aagggcctagtacccagaata

SEQ ID NO: 140 - DNA, 20 bp, synthetic construct
  source 1..20: mol_type = other DNA
gaaccagcagagtggagatg

SEQ ID NO: 141 - DNA, 22 bp, synthetic construct
  source 1..22: mol_type = other DNA
ggcatagatacctgggctaatg

SEQ ID NO: 142 - DNA, 20 bp, synthetic construct
  source 1..20: mol_type = other DNA
caagagaaagcgggtctcgg

SEQ ID NO: 143 - DNA, 20 bp, synthetic construct
  source 1..20: mol_type = other DNA
acattggccattgcacgagt

SEQ ID NO: 144 - DNA, 22 bp, synthetic construct
  source 1..22: mol_type = other DNA
cctctggctaccatccttattc

SEQ ID NO: 145 - DNA, 22 bp, synthetic construct
  source 1..22: mol_type = other DNA
cggtctccagtattagcatctg

SEQ ID NO: 146 - DNA, 22 bp, synthetic construct
  source 1..22: mol_type = other DNA
gtaacccagggaggtcaataaa

SEQ ID NO: 147 - DNA, 23 bp, synthetic construct
  source 1..23: mol_type = other DNA
gcctctacatcactagcactaac

SEQ ID NO: 148 - DNA, 21 bp, synthetic construct
  source 1..21: mol_type = other DNA
gctggtagaagaggccattag

SEQ ID NO: 149 - DNA, 20 bp, synthetic construct
  source 1..20: mol_type = other DNA
ggtgctgacatctggatgaa

SEQ ID NO: 150 - DNA, 20 bp, synthetic construct
  source 1..20: mol_type = other DNA
gcagggaaactgctgatagt

SEQ ID NO: 151 - DNA, 20 bp, synthetic construct
  source 1..20: mol_type = other DNA
gtggaatccaggcagctatt

SEQ ID NO: 152 - DNA, 21 bp, synthetic construct
  source 1..21: mol_type = other DNA
gacagaggatcaagggcttta

SEQ ID NO: 153 - DNA, 23 bp, synthetic construct
  source 1..23: mol_type = other DNA
agaaggctctcaccttagagata

SEQ ID NO: 154 - DNA, 21 bp, synthetic construct
  source 1..21: mol_type = other DNA
gaggaaggtgtgtggagattc

SEQ ID NO: 155 - DNA, 22 bp, synthetic construct
  source 1..22: mol_type = other DNA
gcctaggtggttgagagtaatg

SEQ ID NO: 156 - DNA, 20 bp, synthetic construct
  source 1..20: mol_type = other DNA
gcattcatgaccaggatcac

SEQ ID NO: 157 - DNA, 21 bp, synthetic construct
  source 1..21: mol_type = other DNA
caaagaagatctctgggcgta

SEQ ID NO: 158 - DNA, 20 bp, synthetic construct
  source 1..20: mol_type = other DNA
tgggaccagcaaaggttatt

SEQ ID NO: 159 - DNA, 21 bp, synthetic construct
  source 1..21: mol_type = other DNA
gatcccatcctcacagtgttt

SEQ ID NO: 160 - DNA, 22 bp, synthetic construct
  source 1..22: mol_type = other DNA
ggacgaagactcagcagtttat

SEQ ID NO: 161 - DNA, 23 bp, synthetic construct
  source 1..23: mol_type = other DNA
caggctgctataagtggatgtaa

SEQ ID NO: 162 - DNA, 21 bp, synthetic construct
  source 1..21: mol_type = other DNA
gttcctggtcactgtctcttt

SEQ ID NO: 163 - DNA, 22 bp, synthetic construct
  source 1..22: mol_type = other DNA
gctcctccagataccacataag

SEQ ID NO: 164 - DNA, 20 bp, synthetic construct
  source 1..20: mol_type = other DNA
actggattcctcagcagcac

SEQ ID NO: 165 - DNA, 20 bp, synthetic construct
  source 1..20: mol_type = other DNA
gcctgagcagaaggtgtagg

SEQ ID NO: 166 - DNA, 20 bp, synthetic construct
  source 1..20: mol_type = other DNA
gacctcgtgaaggacaacgg

SEQ ID NO: 167 - DNA, 20 bp, synthetic construct
  source 1..20: mol_type = other DNA
ttgacgatcacggcgagaat

SEQ ID NO: 168 - DNA, 20 bp, synthetic construct
  source 1..20: mol_type = other DNA
gaatctcccaggcggtattt

SEQ ID NO: 169 - DNA, 21 bp, synthetic construct
  source 1..21: mol_type = other DNA
cctcttcctttcggaggattt

SEQ ID NO: 170 - DNA, 20 bp, synthetic construct
  source 1..20: mol_type = other DNA
gcataccacctgccctttat

SEQ ID NO: 171 - DNA, 23 bp, synthetic construct
  source 1..23: mol_type = other DNA
cctttgctttgctctgttcttag

SEQ ID NO: 172 - DNA, 22 bp, synthetic construct
  source 1..22: mol_type = other DNA
caggtcgttctgatagtgaagg

SEQ ID NO: 173 - DNA, 22 bp, synthetic construct
  source 1..22: mol_type = other DNA
cttgtgggactgatttcgtttg

SEQ ID NO: 174 - DNA, 20 bp, synthetic construct
  source 1..20: mol_type = other DNA
gggagacatcgacgagatgc

SEQ ID NO: 175 - DNA, 20 bp, synthetic construct
  source 1..20: mol_type = other DNA
caggaaaggagctacacagc

SEQ ID NO: 176 - DNA, 19 bp, synthetic construct
  source 1..19: mol_type = other DNA
ccatcaccggcagaacaca

SEQ ID NO: 177 - DNA, 20 bp, synthetic construct
  source 1..20: mol_type = other DNA
agstgcagagcattgactcc

SEQ ID NO: 178 - DNA, 20 bp, synthetic construct
  source 1..20: mol_type = other DNA
tgcaccaccaactgcttagc

SEQ ID NO: 179 - DNA, 21 bp, synthetic construct
  source 1..21: mol_type = other DNA
ggcatggactgtggtcatgag

SEQ ID NO: 180 - DNA, 20 bp, synthetic construct
  source 1..20: mol_type = other DNA
gcattcatgaccaggatcac

SEQ ID NO: 181 - DNA, 21 bp, synthetic construct
  source 1..21: mol_type = other DNA
caaagaagatctctgggcgta

SEQ ID NO: 182 - DNA, 20 bp, synthetic construct
  source 1..20: mol_type = other DNA
cagagctcctacccactgct

SEQ ID NO: 183 - DNA, 20 bp, synthetic construct
  source 1..20: mol_type = other DNA
cttctccttctccagcacca

SEQ ID NO: 184 - DNA, 20 bp, synthetic construct
  source 1..20: mol_type = other DNA
catgtgtgtccagcctgaat

SEQ ID NO: 185 - DNA, 20 bp, synthetic construct
  source 1..20: mol_type = other DNA
ggcacagtcgatacactgga

SEQ ID NO: 186 - DNA, 22 bp, synthetic construct
  source 1..22: mol_type = other DNA
ctctacatctgcaaggtggagc

SEQ ID NO: 187 - DNA, 23 bp, synthetic construct
  source 1..23: mol_type = other DNA
agaggaggaagtcagaatctggg

SEQ ID NO: 188 - DNA, 20 bp, synthetic construct
  source 1..20: mol_type = other DNA
cacctggagctgtaccagaa

SEQ ID NO: 189 - DNA, 20 bp, synthetic construct
  source 1..20: mol_type = other DNA
tgcagtgtgttatccctgct

SEQ ID NO: 190 - DNA, 20 bp, synthetic construct
  source 1..20: mol_type = other DNA
atattggggcttgcctttct

SEQ ID NO: 191 - DNA, 20 bp, synthetic construct
  source 1..20: mol_type = other DNA
gtgtaattccagggggaggt

SEQ ID NO: 192 - DNA, 21 bp, synthetic construct
  source 1..21: mol_type = other DNA
agagaaatgagtgagtgtggg

SEQ ID NO: 193 - DNA, 21 bp, synthetic construct
  source 1..21: mol_type = other DNA
acaggaggtgttcccctttgc

SEQ ID NO: 194 - DNA, 20 bp, synthetic construct
  source 1..20: mol_type = other DNA
aggcagggagtgtgtgaact

SEQ ID NO: 195 - DNA, 20 bp, synthetic construct
  source 1..20: mol_type = other DNA
gtggttgtggtggtctgaca

SEQ ID NO: 196 - DNA, 18 bp, synthetic construct
  source 1..18: mol_type = other DNA
caccaccaactgcttagc

SEQ ID NO: 197 - DNA, 21 bp, synthetic construct
  source 1..21: mol_type = other DNA
ggcatggactgtggtcatgag

SEQ ID NO: 198 - DNA, 20 bp, synthetic construct
  source 1..20: mol_type = other DNA
gcattcatgaccaggatcac

SEQ ID NO: 199 - DNA, 21 bp, synthetic construct
  source 1..21: mol_type = other DNA
caaagaagatctctgggcgta

SEQ ID NO: 200 - DNA, 19 bp, synthetic construct
  source 1..19: mol_type = other DNA
ccaggacagaccacattca

SEQ ID NO: 201 - DNA, 20 bp, synthetic construct
  source 1..20: mol_type = other DNA
ctggacacccattccagact

(19) United States
(12) Patent Application Publication
(10) Pub. No.: US 2025/0361485 A1
(43) Pub. Date: Nov. 27, 2025

(54) MODIFIED CD4+ T CELLS EXPRESSING IL-37 AND METHODS OF USE THEREOF

(71) Applicant: The Regents of the University of Colorado, A Body Corporate, Denver, CO (US)

(72) Inventors: Mayumi FUJITA, Englewood, CA (US); Douglas OSBORNE, Denver, CO (US); Maki NAKAYAMA, Aurora, CO (US)

(21) Appl. No.: 18/996,444

(22) PCT Filed: Jul. 18, 2023
(86) PCT No.: PCT/US2023/070445

§ 371 Date: Jan. 17, 2025

Related U.S. Application Data
(60) Provisional application No. 63/390,259, filed on Jul. 18, 2022.

(51) Int. Cl. C12N 5/0783; A61K 35/17; A61K 40/11; A61K 40/35; A61K 40/50; A61P 37/06

(52) U.S. Cl. CPC C12N 5/0637; A61K 35/17; A61K 40/11; A61K 40/35; A61K 40/50; A61P 37/06; C12N 2501/23

(57) ABSTRACT

Disclosed herein are methods of producing a population of modified CD4+T cells expressing nuclear IL-37. Also disclosed are methods of using the modified CD4+T cells expressing nuclear IL-37 for the treatment of immune or inflammatory diseases, disorders, or conditions.

### RELATED APPLICATIONS

This application claims priority to, and the benefit of, U.S. Provisional Application 63/390,259, filed Jul. 18, 2022, the entire contents of which are incorporated herein by reference.

### STATEMENT REGARDING FEDERALLY SPONSORED RESEARCH

This invention was made with government support under 1R01AI156534-01A1 awarded by the National Institute of Health and under 5I01BX001228-10 awarded by the U.S. Department of Veterans Affairs. The government has certain rights in the invention.

### INCORPORATION BY REFERENCE OF SEQUENCE LISTING

The Sequence Listing XML associated with this application is provided electronically in XML file format and is hereby incorporated by reference in its entirety into the specification. The name of the XML file containing the Sequence Listing XML is “UNCO-047_001WO_SeqList_ST26.xml”. The XML file is 178,344 bytes in size, created on Jul. 17, 2023.

### TECHNICAL FIELD

The disclosure is directed to modified CD4+ T cell compositions (i.e., engineered CD4+ T cells), and methods of making and using the same for the adoptive therapy and treatment of immune or inflammatory diseases, disorders, or conditions.

### BACKGROUND

Immune tolerance is vital in preventing immune dysregulation, autoimmunity, and immune rejection. Among cell populations known to be responsible for immune tolerance are regulatory T (Treg) cells. Treg cells play a vital role in suppressing immune responses, inducing tolerance and maintaining homeostasis. Active suppression by Treg cells plays an important role in the down-regulation of T cell responses to foreign and self-antigens. Because FOXP3+ Treg cells play an essential role in the modulation of immune responses and maintenance of peripheral self-tolerance, these cells have the potential to be used as a tool to treat autoimmunity and prevent transplant rejection. Expression of the transcription factor forkhead box P3 (FOXP3) is critical for Treg suppressive function, and while the continual expression of FOXP3 stabilizes Treg cell populations and preserves tolerance, the loss of FOXP3 results in dysfunctional Treg cells. The loss of FOXP3 expression can lead to immune imbalance, tissue injury, and the therapeutic failure of autoimmunity and transplantation.

FOXP3 expression in human Treg cells is dependent on the anti-inflammatory cytokine Interleukin-37 (IL-37). Human Treg cells express the highest IL-37 mRNA levels among blood cells isolated from healthy human individuals. However, the instability of Treg cells presents a problem for current immunotherapies using the adoptive transfer of Treg cells. Following the adoptive transfer, Treg cells can convert to inflammatory T cells, exacerbating the disease. Under inflammatory or pathogenic circumstances, some Treg cells lose FOXP3 expression and/or their suppressive function. Accordingly, there exists a long-felt and unmet need in the art for improved methods for producing and sustaining stable, immunosuppressive FOXP3+ Treg cells for improved immunotherapies.

Disclosed herein are methods for producing a population of modified CD4+ T cells that express IL-37, an anti-inflammatory cytokine critical in maintaining the immunosuppressive function of Treg cells and inducing Treg-like phenotype in non-Treg CD4+ T cells. High expression of IL-37 in the population of modified Treg cells disclosed herein produces a stable Treg cell. High expression of IL-37 in the population of disease-related CD4+ T cells disclosed herein produces non-pathogenic T cell or potentially a Treg cell. The present disclosure provides methods for producing a population of modified CD4+ T cells expressing IL-37. The present disclosure also provides methods of treating an immune-mediated health condition or disorder using the population of modified CD4+ T cells.

### SUMMARY

The disclosure provides a method of producing a population of modified CD4+ T cells comprising introducing into a plurality of human T cells, a composition comprising an Interleukin-37 (IL-37) or a nucleic acid sequence encoding the IL-37 under suitable conditions that express the IL-37 in the nucleus of the human T cell, thereby producing a plurality of modified CD4+ T cells.

The disclosure also provides composition comprising a population of modified CD4+ T cells produced by the methods described herein.

The disclosure also provides a method of treating an immune disease or disorder or an inflammatory disease or disorder comprising administering to a subject in need thereof a therapeutically effective amount of a composition comprising a population of modified CD4+ T cells that express nuclear IL-37.

In some embodiments, the population of modified CD4+ T cells are regulatory T cells or effector T cells. In some embodiments, the CD4+ cells are non-regulatory T cells. In some embodiments, the cells are T conv cells. In some embodiments, the population of modified CD4+ T cells are regulatory T cells.

In some embodiments, the nuclear expression of IL-37 in the plurality of modified CD4+ T cells is at least about 1-fold, at least about 2-fold, at least about 3-fold, at least about 4-fold, at least about 5-fold, at least about 6-fold, at least about 7-fold, at least about 8-fold, at least about 9-fold or at least about 10-fold greater than the nuclear expression of IL-37 in a population of wildtype human T cells. In some embodiments, the nuclear expression of IL-37 in the plurality of modified CD4+ T cells is at least about 5-fold greater than the nuclear expression of IL-37 in a population of wildtype human T cells.

In some embodiments, the nuclear expression of IL-37 in the plurality of modified CD4+ T cells is about 1-fold to 2-fold, about 1-fold to 3-fold, about 1-fold to 4-fold, about 1-fold to 5-fold, about 1-fold to 6-fold, about 1-fold to 7-fold, about 1-fold to 8-fold, about 1-fold to 9-fold, about 1-fold to 10-fold, about 2-fold to 3-fold, about 2-fold to 4-fold, about 2-fold to 5-fold, about 2-fold to 6-fold, about 2-fold to 7-fold, about 2-fold to 8-fold, about 2-fold to 9-fold, about 2-fold to 10-fold, about 3-fold to 4-fold, about 3-fold to 5-fold, about 3-fold to 6-fold, about 3-fold to 7-fold, about 3-fold to 8-fold, about 3-fold to 9-fold, about 3-fold to 10-fold, about 4-fold to 5-fold, about 4-fold to 6-fold, about 4-fold to 7-fold, about 4-fold to 8-fold, about 4-fold to 9-fold, about 4-fold to 10-fold, about 5-fold to 6-fold, about 5-fold to 7-fold, about 5-fold to 8-fold, about 5-fold to 9-fold, about 5-fold to 10-fold, about 6-fold to 7-fold, about 6-fold to 8-fold, about 6-fold to 9-fold, about 6-fold to 10-fold, about 7-fold to 8-fold, about 7-fold to 9-fold, about 7-fold to 10-fold, about 8-fold to 9-fold, about 8-fold to 10-fold, about 9-fold to 10-fold greater than the nuclear expression of IL-37 in a population of wildtype human T cells. In some embodiments, the nuclear expression of IL-37 in the plurality of modified CD4+ T cells is about 5-fold to about 10-fold greater than the nuclear expression of IL-37 in a population of wildtype human T cells.

In some embodiments, the expression of nuclear IL-37 in the population of modified CD4+ T cells is at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or at least about 100% greater than the expression of nuclear IL-37 in a population of wildtype CD4+ T cells. In some embodiments, the expression of nuclear IL-37 in the population of modified CD4+ T cells is at least about 50% greater than the expression of nuclear IL-37 in a population of wildtype CD4+ T cells. In some embodiments, the expression of nuclear IL-37 in the population of modified CD4+ T cells is at least about 85% greater than the expression of nuclear IL-37 in a population of wildtype CD4+ T cells.

In some embodiments, the expression of nuclear IL-37 in the population of modified CD4+ T cells is about 10% to 20%, about 10% to 30%, about 10% to 40%, about 10% to 50%, about 10% to 60%, about 10% to 70%, about 10% to 80%, about 10% to 90%, about 10% to 100%, about 20% to 30%, about 20% to 40%, about 20% to 50%, about 20% to 60%, about 20% to 70%, about 20% to 80%, about 20% to 90%, about 20% to 100%, about 30% to 40%, about 30% to 50%, about 30% to 60%, about 30% to 70%, about 30% to 80%, about 30% to 90%, about 30% to 100%, about 40% to 50%, about 40% to 60%, about 40% to 70%, about 40% to 80%, about 40% to 90%, about 40% to 100%, about 50% to 60%, about 50% to 70%, about 50% to 80%, about 50% to 90%, about 50% to 100%, about 60% to 70%, about 60% to 80%, about 60% to 90%, about 60% to 100%, about 70% to 80%, about 70% to 90%, about 70% to 100%, about 80% to 90%, about 80% to 100%, about 90% to 100% greater than the expression of nuclear IL-37 in a population of wildtype CD4+ T cells. In some embodiments, the expression of nuclear IL-37 in the population of modified CD4+ T cells is about 50% to about 80% greater than the expression of nuclear IL-37 in a population of wildtype CD4+ T cells.

In some embodiments, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the plurality of modified CD4+ T cells express at least one marker of a regulatory T cell. In some embodiments, at least about 75% of the plurality of modified CD4+ T cells express at least one marker of a regulatory T cell. In some embodiments, at least about 95% of the plurality of modified CD4+ T cells express at least one marker of a regulatory T cell.

In some embodiments, at least one marker of a regulatory T cell is selected from a group consisting of FOXP3, CD25, CD4, CTLA4, IL-10, GITR, TGF-beta and CD127. In some embodiments, the least one marker is FOXP3. In some embodiments, at least one marker is FOXP3 and CD25.

In some embodiments, the population of modified CD4+ T cells are allogeneic CD4+ T cells. In some embodiments, the population of modified CD4+ T cells are autologous CD4+ T cells.

In some embodiments, the treatment of an immune disease or disorder selected from the group consisting of: allergic contact hypersensitivity, graft versus host disease, transplant rejection, type 1 diabetes, systemic lupus erythematosus, inflammatory bowel disease, Crohn's disease, ulcerative colitis and multiple sclerosis. In some embodiments, the immune disease or disorder is allergic contact hypersensitivity. In some embodiments, the immune disease or disorder is graft versus host disease. In some embodiments, the immune disease or disorder is inflammatory bowel disease. In some embodiments, the immune disease or disorder is type 1 diabetes.

In some embodiments, the treatment of an inflammatory disease or disorder selected from the group consisting of: inflammatory diseases or disorders affecting the digestive system, joints, skin, respiratory system, and nervous system.

In some embodiments, the inflammatory disease or disorder is selected from the group consisting of psoriasis, traumatic brain injury, bronchitis and pneumonitis. In some embodiments, the inflammatory disease or disorder is psoriasis. In some embodiments, the inflammatory disease or disorder is traumatic brain injury. In some embodiments, the inflammatory disease or disorder is bronchitis. In some embodiments, the inflammatory disease or disorder is pneumonitis.

### BRIEF DESCRIPTION OF THE DRAWINGS

FIGS. 1A-F show a series of graphs depicting that IL-37 is highly expressed in human T regulatory cells. FIG. 1A shows a series of contour plots depicting a flow cytometry gating strategy to analyze cell surface protein and IL-37 mRNA expression in myeloid cells. FIG. 1B shows a series of contour plots depicting a flow cytometry gating strategy to analyze cell surface protein and IL-37 mRNA expression in lymphoid cells. FIG. 1C shows two pie charts depicting the percentage of cellular subsets among IL37-expressing cells from five healthy human donors. The left chart shows unstimulated cells. The right chart shows cells stimulated with 100 ng/ml LPS for 24 hours. Cellular subsets and percentages are as indicated in the pie charts. mDC: myeloid-derived dendritic cell; pDC: plasmacytoid dendritic cell; NK: natural killer cells. FIG. 1D shows a graph depicting the frequency of IL-37 mRNA-expressing cells in the myeloid (left) and lymphoid (right) subset in the absence (−) or presence (+) of LPS. Cellular subsets are as indicated in the legend. mDC: myeloid-derived dendritic cell; pDC: plasmacytoid dendritic cell; NK: natural killer cells. FIG. 1E shows a series of histograms depicting IL-37 expression in myeloid (left) and lymphoid (right) cell subsets that were unstimulated (top) or treated with 100 ng/ml LPS (bottom) for 24 hours. Cellular subsets are as indicated in the legend. mDC: myeloid-derived dendritic cell; pDC: plasmacytoid dendritic cell; NK: natural killer cells. FIG. 1F shows a graph depicting the mean fluorescent intensity (MFI) of IL37 mRNA expression in the unstimulated and LPS-treated cellular subsets depicted in FIG. 1E. Cellular subsets are as depicted in the legend.

FIG. 2 shows a schematic of a pLenti-IL37-C-Myc-DDK-P2A-Puro vector (IL37 OE) (Origene) transduced into Jurkat cells for the expression of IL37.

FIGS. 3A-E show a series of graphs depicting that IL37 overexpression induces FOXP3 in Jurkat cells using the vector depicted in FIG. 2. FIG. 3A shows bar graphs of IL37 mRNA expression in Jurkat cells transfected with an empty vector control or IL37 OE Jurkat cells (IL37 OE). GAPDH served as an internal control, and the values represent IL37 gene expression as a ratio to GAPDH expression. Expression measured using qRT-PCR. FIG. 3B shows IL-37 and FOXP3 immunoblotting of empty vector control and IL37 OE Jurkat cells. Actin was used as an internal control. Gray bars depict IL37 OE Jurkat cells. Black bars depict vector control. Representative immunoblotting (left panels) and quantification of bands (right panels) of three immunoblot experiments. The band densities of proteins were quantified with Image J, normalized to actin, and expressed as fold changes compared to control Jurkat cells expressing an empty vector. FIG. 3C shows bar graphs of FOXP3 mRNA expression in vector control and IL37 OE Jurkat cells. GAPDH served as an internal control, and the values represent FOXP3 gene expression as a ratio to GAPDH expression. FIGS. 3D-E shows histograms (FIG. 3D) and bar graphs depicting densitometry measurement quantification (FIG. 3E) of FOXP3 protein expression in vector control and IL37 OE Jurkat cells. IgG used for negative control staining (solid gray histogram). Expression is presented as mean fluorescent intensity (MFI) in IL37 OE Jurkat cells compared to vector control. Small horizontal lines indicate mean±s.e.m. *P<0.05; **P<0.01; ***P<0.001; ****P<0.0001 (Student's t-test). Data are representative of three independent experiments.

FIGS. 4A-H show a series of graphs depicting IL37 overexpressing (IL37 OE) Jurkat cells are phenotypically similar to T regulatory (Treg) cells. FIG. 4A shows a bar graph depicting qRT-PCR analysis of CTLA-4 mRNA expression in IL37 OE Jurkat cells compared to vector control. FIG. 4B shows a bar graph depicting qRT-PCR analysis of IL-10 mRNA expression in IL37 OE Jurkat cells compared to vector control. FIG. 4C shows a bar graph depicting qRT-PCR analysis of GITR mRNA expression in IL37 OE Jurkat cells compared to vector control. FIG. 4D shows a bar graph depicting qRT-PCR analysis of TGFB mRNA expression in IL37 OE Jurkat cells compared to vector control. In each of FIGS. 4A-D, GAPDH served as an internal control, and the values represent the gene expression level as a ratio to GAPDH expression. FIG. 4E shows a histogram depicting CTLA-4 expression in IL37 OE Jurkat cells compared to vector control. IgG was used for negative control staining. FIG. 4F shows quantification by mean fluorescent intensity (MFI) of the CTLA-4 expression depicted in FIG. 4E. FIG. 4G shows a series of contour plots depicting IL-10+ cell gating in unstimulated (unstim) and anti-CD3/CD28-treated (+CD3/CD28) IL37 OE Jurkat cells compared to vector control cells. IgG was used for negative control staining. FIG. 4H shows a bar graph depicting the quantification of IL-10 expression in Jurkat cells shown in FIG. 4G. Small horizontal lines indicate mean±s.e.m. *P<0.05; **P<0.01; ***P<0.001; ****P<0.0001 (Student's t-test). Data are representative of four independent experiments.

FIGS. 5A-C show a series of graphs depicting that IL-37 overexpressing (IL37 OE) Jurkat cells are highly suppressive in vitro T cell suppression assay. FIG. 5A and FIG. 5B show a series of graphs of in vitro T cell suppression assay by vector control and IL37 OE Jurkat cells. Histograms (FIG. 5A) and % proliferation (FIG. 5B) showing division of CFSE-labeled CD4+CD25− T cell responders (Tresp) purified from healthy human donor PBMCs, cultured with anti-CD3 and either vector control Jurkat cells (black bars), IL37 OE Jurkat cells (gray bars), or human primary Treg cells (white bars) at Treg:Tresp ratios of 4:1 to 1:2 for 5 days. Purified Tresp cells without Jurkat cells or Treg cells (0:1) were used as a positive control of Tresp proliferation without suppression (an orange bar). The x-axis depicts the ratio of Jurkat cells to Tresp cells. The y-axis depicts percent proliferation. Black bars depict vector control Jurkat cells. FIG. 5C shows bar graphs of ELISA analysis of IL-37 in the supernatant of vector control and IL37 OE Jurkat cells. Recombinant human IL-37 controls at 2 ng/ml and 1 ng/ml were used as positive controls. Data represents mean±s.e.m. NS, not significant (P>0.05); *P<0.05; **P<0.01; ***P<0.001; ****P<0.0001 (Student's t-test). Data are representative of two independent experiments.

FIGS. 6A-B show a series of graphs depicting increased FOXP3 expression in transgenic (Tg) Treg cells expressing IL-37. FIG. 6A shows a histogram depicting FOXP3 expression in IL-37 transgenic (Tg) Treg cells compared wildtype (WT) and isotype control. FIG. 6B shows a bar graph depicting the quantification of FOXP3 expression as presented in FIG. 6A. Expression is presented as mean fluorescent intensity (MFI).

FIGS. 7A-C show a series of graphs depicting the enhanced suppressive function of transgenic (Tg) Treg cells expressing IL-37 in an in vitro T cell suppression assay. FIG. 7A shows a series of histograms depicting the division of CFSE-labelled T cell responder (Tresp) cells (CD4+CD25−) from WT mice co-incubated with either Treg cells from WT mice or IL37 transgenic (Tg) mice for 3 days at the indicated ratios. FIG. 7B shows a bar graph depicting the proliferation index of the T cell cocultures presented in FIG. 7A. FIG. 7C shows a bar graph depicting the percent (%) suppression of T cell cocultures analyzed in FIG. 7A.

FIGS. 8A-D show a series of graphs and microscopy images depicting IL37 transgenic Treg (IL37 Treg) cells suppressing skin inflammation in vivo using a mouse model. FIG. 8A shows a schematic depicting an exemplary mouse contact hypersensitivity (CHS) model. Either donor IL-37 Transgenic (Tg) Treg cells or donor WT Treg cells are adoptively transferred into WT mice (Recipient) following hapten sensitization (DNFB) and prior to a subsequent hapten challenge (DNFB). DNFB is Dinitrofluorobenzen. FIG. 8B shows a line graph depicting ear swelling in mice administered IL-37 Tg-Treg cells compared to WT Treg cells and vehicle controls in the CHS model depicted in FIG. 8A. Treatments are as indicated in the legend. FIG. 8C shows a series of H&E microscopy images of ear specimens taken 48 hours after the DNFB challenge in the mouse model depicted in FIG. 8A. Ear specimens were taken from mice receiving either vehicle or DNFB treatment and adoptive transfer of cells as indicated. Scale bar, 100 μm. FIG. 8D shows a series of contour plots (left panels) and quantification (right) of CFSE+CD3+ T cells present in the DNFB-treated ears of mice adoptively transferred with either PBS, WT Treg or IL37 Tg Treg cells. Contour plots from the ears of mice injected with PBS and challenged with DNFB were used as a negative control for CFSE gating. Numbers in contour plots depict percentages of cells. Each symbol represents an individual mouse; small horizontal lines indicate mean±s.e.m. (n=5-8 mice per group) (FIGS. 8A-D). Data represent mean±s.e.m. *p<0.05; **p<0.01; ***p<0.001. Statistics in (FIG. 8B) depict the comparison to mice injected with PBS and challenged with DNFB (Student's t-test). Data are representative of two experiments.

FIGS. 9A-D show a series of graphs depicting the role of elevated IL-37 in preventing Treg cell conversion to Tconv cells in inflammatory conditions in vitro. FIG. 9A shows bar graphs of Foxp3 mRNA expression analyzed via qRT-PCT in WT and IL37 Tg Treg cells cultured for 48 h with anti-CD3/CD28 in the absence (−IL-6) or presence (+IL-6) of 20 ng/ml mouse IL-6. GAPDH served as an internal control. FIG. 9B shows histograms (upper) and quantification (lower) of Foxp3 in WT and IL37 Tg CD4+ T cells cultured for 48 h with anti-CD3/CD28 in the absence (−IL-6) or presence (+IL-6) of 20 ng/ml IL-6. FIG. 9C shows bar graphs of IL37 mRNA in IL37 Tg Treg cells cultured for 48 h with anti-CD3/CD28 in the absence (−IL-6) or presence (+IL-6) of 20 ng/ml mouse IL-6. GAPDH served as an internal control. FIG. 9D shows contour plots (left) and quantification (right) of the percentage of CD25+FOXP3+ Treg cells in IL-37^{hi} and IL-37^{lo} Treg cells cultured for 48 h with anti-CD3/CD28 in the absence (−IL-6) or presence (+IL-6) of 20 ng/ml human IL-6. FIG. 9E shows bar graphs depicting Th lineage gene expression: TBET, GATA, RORγT, and Th17-specific gene expression: STAT3, IRF4, and IL17 in siIL37 human Tregs. GAPDH served as an internal control. FIG. 9F shows bar graphs depicting Th lineage gene expression: Gata, Rorγt Irf4, and Il17 in unstimulated or TCR-stimulated mouse Tregs+/−IL-6. Gapdh served as an internal control. Each symbol represents an individual donor/mouse; small horizontal lines indicate mean±s.e.m. Data represent mean±s.e.m. (n=5-8 mice per group or 3-6 donors per group). NS, not significant (p>0.05); *P<0.05; **P<0.01; and ***P<0.001 (Student's t-test). Data are representative of 2-3 independent experiments. FIG. 9G shows a series of contour plots depicting Treg and Tconv cells from ear specimens taken from mice 48 hours after the DFNB challenge in the CHS model depicted in FIG. 8A. FIG. 9H shows a bar graph depicting quantification (frequency) of Treg and Tconv cells in samples presented in FIG. 9G.

FIGS. 10A-B shows a series of bar graphs depicting gene expression knockdown in human Treg cells 24 hours after transfection with IL-37 siRNA (siIL37) compared to scramble siRNA control (siCtrl). Expression was analyzed by qRT-PCR. FIG. 10A shows the knockdown of IL-37 expression in Treg cells. FIG. 10B shows the knockdown of FOXP3 expression in Treg cells.

FIGS. 11A-F show a series of images and graphs depicting that IL-37 interacts with p-SMAD3 to translocate into the nucleus and induce FOXP3 expression in human Treg cells. FIG. 11A shows a seriew of confocal microscopic images depicting IL-37 localization in Treg compared to Tconv cells. The confocal images depict cells stained with the DNA-intercalating dye DAPI, IL-37 and overlay of IL-37 and DAPI in Treg and Tconv cells. Right, quantification of IL-37 intensity in >100 individual Tconv and Treg cells. Far right, Pearson's coefficient of IL-37 colocalization with DAPI counted using overlay images of IL-37 and DAPI in Tconv and Treg cells. FIG. 11B shows images of immunoblotting of IL-37 on SMAD3 immunoprecipitants of resting Tconv and Treg cells lysates. FIG. 11C shows a table depicting the list of Fullmoon® protein and phosphorylation site antibody array measuring fold change between purified mouse Treg cells from WT and IL37 Tg. FIGS. 11D-E show histograms (FIG. 11D) and quantification (MFI) (FIG. 11E) of CD4 and p-SMAD3 Ser208 from IL-37hi and IL-37^{lo} human Treg cells. FIG. 11F shows confocal microscopic images of immunofluorescence proximity ligation assay (PLA) of IL-37 and p-SMAD3 S208 (dots) in human Tconv and Treg cells; DNA-intercalating dye DAPI. Scale bars, 10 μm. Each symbol represents an individual donor; small horizontal lines indicate mean±s.e.m., (n=4-5 donors per group) NS, not significant (p>0.05); **p<0.01; ***p<0.001 (Student's t-test). Data are representative of 2-4 independent experiments.

FIGS. 12A-B show a series of images and graphs depicting the role of caspase-1 in controlling the nuclear localization of IL-37 and its function in maintaining Foxp3 expression in mouse Treg cells and in vivo immune suppression under inflammatory conditions. FIG. 12A shows confocal microscopic images of unstimulated IL37 Tg and D20A mouse Treg cells. Images of cells stained with IL-37 (AF488), DAPI, and overlay of IL-37 and DAPI. Quantification of nuclear localization of IL-37 from images in (A), shown by Pearson's coefficient of IL-37 colocalization with DAPI (right) counted using overlay images of IL-37 and DAPI in Treg cells. FIG. 12B shows a series of bar graphs of IL37 (left) and FOXP3 (right) in WT, IL37 Tg, and D20A Treg cells. Expression was analyzed by qRT-PCR. GAPDH served as an internal control. Each symbol represents an individual donor; small horizontal lines indicate mean±s.e.m, (n≥100 cells/group) (FIG. 12A), (n=4-6 mice per group) (FIG. 12B). *p<0.05; **p<0.01; ***p<0.001; ****p<0.0001 (Student's t-test). Data are representative of 2-3 independent experiments.

FIGS. 13A-C show a series of images and graphs depicting caspase-1 activity is required for immune suppression. FIG. 13A shows a series of H&E stained microscopic images of ear specimens taken at 48 h after DNFB challenge from mice adoptively transferred with PBS, WT-Treg, IL37 Tg, or D20A Treg. Scale bar, 100 μm. FIG. 13B shows a line graph depicting CHS responses measured by ear thickness at 0, 6, 24, and 48 h after vehicle (vehicle+PBS) or DNFB challenge in WT mice adoptively transferred with PBS (DNFB+PBS), WT Treg cells (DNFB+WT-Treg), IL37 Tg Treg cells (DNFB+IL37 Tg-Treg) or D20A Treg cells (DNFB+D20ATreg). FIG. 13C shows a series of bar graphs depicting the frequency of CFSE-labeled cells among Treg cells (CD4+CD25^{hi}CD45RB^{lo}) (left) and Tconv cells (CD4+CD25^{lo}CD45RB^{hi}) (right) from ear specimens, taken at 48 h after DNFB challenge from mice adoptively transferred with WT-Treg, IL37 Tg Treg or D20A Treg cells. Each symbol represents an individual donor; small horizontal lines indicate mean±s.e.m, (n=4-6 mice per group). *p<0.05; **p<0.01; ***p<0.001; ****p<0.0001 (Student's t-test). Data are representative of 2-3 independent experiments.

FIGS. 14A-E show a series of graphs depicting that IL-37 overexpressing Jurkat cells are highly suppressive in vitro. FIGS. 14A-B shows a series of graphs of in vitro T cell suppression assay by vector control and IL37 OE Jurkat cells. Histograms (FIG. 14A) and % proliferation (FIG. 14B) showing division of CFSE-labeled CD4+CD25-T cell responders (Tresp) purified from healthy human donor PBMCs, cultured with anti-CD3 and either vector control Jurkat cells (black bars), IL37 OE Jurkat cells (gray bars), or human primary Treg cells (white bars) at Treg:Tresp ratios of 4:1 to 1:2 for 5 days. Purified Tresp cells without Jurkat cells or Treg cells (0:1) were used as a positive control of Tresp proliferation without suppression (an orange bar).

FIG. 14C shows bar graphs of ELISA analysis of IL-37 in the supernatant of vector control and IL37 OE Jurkat cells. Recombinant human IL-37 controls at 2 ng/ml and 1 ng/ml were used as positive controls. Data represents mean #s.e.m. NS, not significant (P>0.05); *P<0.05; **P<0.01; ***P<0.001; ****P<0.0001 (Student's t-test). Data are representative of two independent experiments. FIG. 14D shows a series of confocal microscopy images and western blots depicting IL-37 expression in Jurkat cells transduced with either empty vector (Empty), IL-37 vector (IL37), or IL-37D20A vector (D20A). IL-37 was stained with AF488. Cells were stained with DAPI. Western blot was performed on the same cells (Empty, IL37, D20A), as indicated. Cell lysates were probed with IL-37 antibody. Actin was detected as a control. FIG. 14E shows a series of graphs depicting CTLA4 and FOXP3 gene expression via qRT-PCR and FOXP3 surface expression via flow cytometry in empty-Jurkat cells (Empt) and IL-37 expressing Jurkat cells (IL37 or D20A) compared to IgG control (IgG).

FIGS. 15A-F show a series of graphs depicting the role of caspase-1 in controlling the nuclear localization of IL-37 and its function in maintaining FOXP3 expression in human Treg cells. FIG. 15A shows histograms (left) and quantification (MFI) (right) of p-caspase-1 Ser376 in Tconv and Treg cells. FIG. 15B shows flow cytometry-based FAM-FLICA® assay measuring caspase-1 activation in resting Tconv and Treg cells. FIG. 15C shows western blot detection of cleaved caspase-1 (cleaved casp-1) from Tconv and Treg cell lysates. Actin was used as a control. FIG. 15D shows histograms of p-SMAD3 Ser208 in Treg cells+/−the caspase-1 inhibitor Ac-YVAD-CMK. Tconv cells used as a control. FIG. 15E shows confocal microscopic images of unstimulated human Treg cells cultured for 24 h in the absence (−) or presence (+) of 50 μM caspase-1 inhibitor (Ac-YVAD-CMK). Images of cells stained with DAPI, IL-37 (AF488), and overlay of IL-37 and DAPI. Far right, plot profiles of fluorescence intensity, presented in arbitrary units (AU), spanning α to ω (white line in merged image) with lines matching the image stains. Scale bar, 5 μm. FIG. 15F shows histogram (left) and quantification (right) of IL-37 MFI in human Treg cells following 48 h culture in the absence (−) or presence (+) of 50 μM caspase-1 inhibitor (Ac-YVAD-CMK).

FIGS. 16A-D show FOXP3 mRNA (FIG. 16A) and protein (FIG. 16B) expression in human Treg cells, unstimulated (Unstim or -) or stimulated (CD3/CD28 or -) with anti-CD3/CD28 for 48 h in the absence or presence of 50 μM caspase-1 inhibitor (Ac-YVAD-CMK). mRNA expression was evaluated via qRT-PCR (FIG. 16A). Quantification of protein (FIG. 16B, right) is as indicated by values on contour plots (FIG. 16B, left). GAPDH served as an internal control. FIG. 16C shows bar graphs of FOXP3 gene expression following siRNA knockdown of caspase-1 in a population of human CD4+ T cells. Gene expression analyzed by qRT-PCR. GAPDH served as an internal control. FIG. 16D shows contour plots of FOXP3+ cell gating in human CD4+ T cells transfected with scramble siRNA (Scramble) and siCasp-1 48 h prior (left) and quantification of the percentage of FOXP3+ T cells (right). Cell populations on contour plots are identified in boxes. Each symbol represents an individual donor; small horizontal lines indicate mean±s.e.m. (n=4-6 donor per group). **P<0.01; ***P<0.001; ****p<0.0001 (Student's t-test). Data are representative of 2-4 independent experiments.

FIG. 17 shows an confocal microscopy image depicting IL-37 expression and localization in human Treg cells. IL-37 was stained with AF488 (green). Treg cells were stained with DAPI.

FIGS. 18A-C show a series of histograms and bar graphs depicting an in vitro T cell suppression assay of CFSE-labeled CD4+CD25-CD127+ T cell responders (Tresp) cultured with CD4+CD25+CD127-human Treg cells transfected with scrambled siRNA (black) or siIL37 (gray). FIG. 18A shows a series of histograms depicting the division of Tresp cells cultured with human Treg cells transfected with siRNA as indicated, at ratios between 2:1 and 32:1. Tresp cells cultured alone with anti-CD3 for 3 days served as a positive control (Tresp:Treg ratio of 1:0). FIG. 18B shows a bar graph depicting the proliferation indexes of the Tresp cells in the cultures depicted in FIG. 18A. Tresp:Treg cell ratios are as indicated. FIG. 18C shows a bar graph depicting the percent (%) suppression of the Tresp cell cultures as depicted in FIG. 18B. Each symbol represents an individual donor; small horizontal lines indicate mean±s.e.m. (n=3-5 donors per group). Data represent mean±s.e.m. *p<0.05; **p<0.01; ***p<0.001. Data are representative of three independent experiments

FIGS. 19A-D show a series of graphs depicting the mouse T cells and Treg cells from wild-type (WT) mice and IL-37 transgenic (Tg) mice. FIG. 19A shows pseudocolor plots (left) and quantification (right) of thymocytes from 6-8-week-old WT or IL37 Tg mice, stained with anti-CD4, anti-CD8, anti-CD25, and anti-CD45RB antibodies. Numbers in dot plots depict the percentage of cells with CD8 single-positive (SP) cells (top left), CD4 and CD8 double-positive (DP) cells (top right), CD4 and CD8 double-negative (DN) cells (bottom left), and CD4 SP cells (bottom right). FIG. 19B shows quantification of splenocytes, CD4+, and CD4+CD25+ T cells from WT or IL37 Tg mice following purification. FIG. 19C shows a histogram (left) and the bar graph (right) depicting Foxp3 expression in IL37 Tg Treg cells compared to WT Treg cells and an isotype control. Expression is presented as mean fluorescent intensity (MFI). FIG. 19D shows a series of histograms depicting an in vitro T cell suppression assay of CFSE-labeled CD4+CD25-T cell responders (Tresp) from WT mice cultured with anti-CD3 and Treg cells from WT or IL37 Tg mice at Treg:Tresp ratios of 1:2, 1:3, and 1:8 for 3 days. Data represent mean±s.e.m. (n=5-8 mice per group) (FIGS. 19A-D). NS, not significant (P>0.05); *P<0.05 (Student's t-test). Data are representative of 2-3 independent experiments.

FIGS. 20A-E show a series of graphs depicting the role of elevated IL-37 in preventing Treg cell conversion to Tconv cells in inflammatory conditions in vivo. FIG. 20A shows contour plots (top) and frequency (bottom) of CFSE-labeled cells among Treg cells (CD4+CD25^{hi}CD45RB^{lo}) and Tconv cells (CD4+CD25^{lo}CD45RB^{hi}) from ear specimens, taken at 48 h after DNFB challenge from mice adoptively transferred with WT-Treg or IL37 Tg Treg cells. Numbers in dot plots depict percentages of CFSE+ cells in Tconv or Treg cells. FIGS. 20B-E show a series of analyses from ear specimens (FIGS. 20B, 20C) and cervical lymph nodes (FIGS. 20D, 20E) taken at 48 h after DNFB challenge from mice adoptively transferred with PBS (DNFB+PBS), WT-Treg (DNFB+WT-Treg) or IL37 Tg Treg (DNFB+IL37 Tg-Treg). FIGS. 20B and 20D show pie charts depicting the frequency of CD8+, CD4+, and CD8−CD4− (DN) cells in CD3+ cells (upper), as well as Tconv (CD4+CD25^{lo}CD45RB^{hi}) and Treg (CD4+CD25^{hi}CD45RB^{lo}) cells in a population of CD4+ cells (lower). FIGS. 20C and 20E show bar graphs depicting the quantification of cell numbers, CD4+ and CD8+ T cells taken at 48 h after vehicle or DNFB challenge (+) from mice adoptively transferred with PBS, WT-Treg or IL37 Tg Treg cells. Each symbol represents an individual donor/mouse; small horizontal lines indicate mean±s.e.m. (n=3-5 mice/donors per group). *p<0.05; **p<0.01; ***p<0.001 (Student's t-test). Data are representative of two independent experiments.

FIGS. 21A-B show a series of graphs depicting a screening protocol and FOXP3 expression analysis in human Treg cells. FIG. 21A shows the screening protocol to determine which proteins are associated with the FOXP3 transcription factors: NFAT, Smad3, and STAT5, to analyze further their role in FOXP3 expression and human Treg cell function. FIG. 21B shows qRT-PCR analysis in resting Tconv and Treg cells of the remaining 15 of 20 proteins associated with the FOXP3 transcription factors: NFAT, Smad3, and STAT5. GAPDH served as an internal control. Each symbol represents an individual donor; small horizontal lines indicate mean±s.e.m., (n=4 donors per group). NS, not significant (P>0.05); *P<0.05 (Student's t-test). Data are representative of four independent experiments.

FIGS. 22A-C show a series of graphs depicting that FOXP3 does not control IL-37 expression in human Treg cells. FIG. 22A shows qRT-PCR analysis of FOXP3 (left) and IL37 (right) in human Treg cells 24 h following transfection with scramble siRNA (Ctrl) or pooled siRNA against FOXP3. Human Tconv cells were used as a reference for negative FOXP3 expression. GAPDH served as an internal control. FIGS. 22B-C show the expression of CTLA-4 (FIG. 22B) and IL-37 (FIG. 22C) in human Treg cells transfected with scramble siRNA (Ctrl) or pooled siRNA against FOXP3 48 h prior, followed by culturing in the absence (Unstim or -) or presence (CD3/CD28 or +) of anti-CD3/CD28 for 24 h. Histogram of unstimulated (left) and stimulated (middle) Treg cells and quantification (right). Tconv cells were used as a reference. Each symbol represents an individual donor; small horizontal lines indicate mean±s.e.m. (n=3-5 donors per group) (FIGS. 22A-C). NS, not significant (P>0.05); *P<0.05, **P<0.01 (Student's t-test). Data are representative of three independent experiments.

FIGS. 23A-C show a series of graphs depicting IL37-expressing cells in human PBMCs using a flow cytometry-based PrimeFlow® RNA assay. FIGS. 23A-C show histogram (FIG. 23A) and quantification by frequency (FIG. 23B) and mean fluorescent intensity (MFI) (FIG. 23C) of IL37 mRNA expression in myeloid and lymphoid cell subsets. PBMCs were treated in the absence (−, black lines or closed symbols) or presence (+, gray lines or open symbols) of 100 ng/ml LPS for 24 h. A gray histogram represents the fluorescence of the label probe only (background) for each immune cell subset as a negative control. Gating for determining positive cell frequency was established to exclude ˜99% of the control events as negative. RPL13a mRNA served as an internal control (not shown). Each symbol represents an individual donor; small horizontal lines indicate mean #s.e.m., (n=5 donors per group) (FIGS. 23A-C). NS, not significant (P>0.05); *P<0.05; **P<0.01; ***P<0.001 (Student's t-test). Data are representative of two independent experiments.

FIGS. 24A-D show a series of images and graphs depicting that IL-37 interacts with p-SMAD3 S208 to translocate into the nucleus and induce FOXP3 expression in human Treg cells. FIG. 24A shows a schematic of SMAD3 phosphorylation and IL-37 interaction. FIG. 24B shows bar graphs of FOXP3 in purified human Treg cells treated with Smad3 inhibitors SIS3 and flavopridol. GAPDH served as an internal control. FIG. 24C shows pseudo color plots of FOXP3 expression in control, SMAD3 inhibitors SIS3 and flavopiridol treated purified human CD4+ T cells+/−TGF-β. FIG. 24D shows Western blot analyses of SMAD3 phosphorylation, IL-37 and FOXP3 expression in the nucleus and cytoplasm of human Tregs treated with flavopiridol. Tubulin is used as cytoplasmic control, and FOXP3 is used as a nuclear control. Each symbol represents an individual donor; small horizontal lines indicate mean±s.e.m., (n=3-5 donors per group) **p<0.01. (Student's t-test). Data are representative of 2-4 independent experiments.

FIG. 25A-J show a series of graphs depicting NLRC4 inflammasome and upstream signaling required for IL-37-directed FOXP3 expression in human Treg cells. FIGS. 25A-B shows a heat map/clustergram (FIG. 25A) and scatterplots (FIG. 25B) of inflammasome pathways and components of mRNA expression in purified human Tconv and Treg cells using the GeneGlobe Data Analysis Center on QIAGEN's website at http://www.qiagen.com/kr/shop/genes-and-pathways/data-analysis-center-overview-page/. Qiagen RT2 Profiler PCR Inflammasome array used to measure the expression of 84 genes associated with inflammasomes, according to the manufacturer's instructions. Scatterplots depicting 2-fold boundary for expression of inflammasome genes in Tconv and Treg cells. FIG. 25C shows bar graphs of NAIP expression in unstimulated Tconv and Treg cells. FIG. 25D shows histograms (upper) depicting expression in Tconv (black) and Treg (gray) cells and quantification (lower). FIG. 25E shows immunoblotting of NLRC4 in Tconv and scramble control and siNLRC4 transfected primary human CD4+CD25+CD127^{dim} Treg cells. FIG. 25F shows histograms (left) and quantification (MFI) (right) of p-NLRC4 S533 in Tconv and Treg cells. FIG. 25G shows flow cytometry-based FAM-FLICA® assay measuring caspase-1 activation in scramble control and siASC, siNLRP2, and siNLRC4 transfected CD4+CD25+CD127^{dim} Treg cells, as well as their quantification of the % caspase-1+ Treg cells (far right). FIG. 25H shows bar graphs of FOXP3 gene expression following siRNA knockdown of NLRC4 in purified CD4+CD25+CD127^{dim} Treg cells. GAPDH served as an internal control. FIG. 25I shows pseudo-color plots (left) showing the percentage of CD4+FOXP3+ Treg cells in a population of purified CD4+ T cells 48 h following siRNA knockdown of NLRC4, and its quantification of the % FOXP3+CD4+ T cells (right). FIG. 25J shows confocal microscopic images of the proximity ligation assay of ASC and NLRC4 (dots) in Treg cells; DNA-intercalating dye DAPI. Scale bars, 5 μm. Small horizontal lines indicate mean±s.e.m, (n=3 donors per group) (FIG. 25A, FIG. 25B), (n=5-6 donors per group) (FIG. 25 C-I), and (n≥100 cells/group (FIG. 25J)). *p<0.05; ***p<0.001, ****p<0.0001 (Student's t-test). Data are representative of 2-3 independent experiments.

FIGS. 26A-G show a series of graphs depicting that basal PAK1/2/3 signaling promotes NLRC4 inflammasome activation in human Treg cells. FIG. 26A shows histograms (left) and quantification (MFI) (right) of p-PAK1/2/3 in purified CD4+CD25+CD127^{dim} Treg cells treated for 24 hrs with DMSO or 1 μM FRAX 597. FIG. 26B shows immunoblotting images of activated group 1 PAKs in purified CD4+CD25+CD127^{dim} Treg cells treated for 48 hrs with DMSO or 1 μM FRAX 597. FIG. 26C shows histograms (left) and quantification (MFI) (right) of p-NLRC4 S533 in purified CD4+CD25+CD127^{dim} Treg cells treated for 24 hrs with DMSO or 1 μM FRAX 597. FIG. 26D shows flow cytometry-based FAM-FLICA® assay measuring caspase-1 activation in purified CD4+CD25+CD127^{dim} Treg cells treated for 24 hrs with DMSO or 1 μM FRAX 597. FIG. 26E shows confocal microscopic images of IL-37 localization in Treg cells treated for 24 hrs with DMSO (Top) or 1 μM FRAX 597 (Bottom). Scale bars, 10 μm. FIG. 26F shows bar graphs of FOXP3 gene expression in Tconv and Treg cells treated for 24 hrs with DMSO or 1 μM FRAX 597. GAPDH served as an internal control. FIG. 26G shows immunoblotting images of FOXP3 in Treg cells treated for 48 hrs with DMSO or 1 μM FRAX 597. Small horizontal lines indicate mean±s.e.m, (n=4-6 donors per group), (n>100 cells/group) (FIG. 26E). *p<0.05; **p<0.01; ****p<0.0001 (Student's t-test). Data are representative of 3-4 independent experiments.

FIGS. 27A-C show a series of graphs depicting differences between expanded WT Treg cells and expanded IL37 Treg cells. FIG. 27A shows a line graph depicting WT and IL37 Treg cell counts over the expansion assay time scale. FIG. 27B shows a series of graphs depicting in vitro T cell suppression assay using expanded WT or IL37 primary mouse CD4+CD25+CD127− Treg cells. Histograms (top), proliferation indexes (left, bottom) and % suppression (right, bottom) showing division of CFSE-labeled CD4+CD25-CD127+ T cell responders (Tresp), cultured with irradiated APCs and anti-CD3 and Treg cells at Tresp:Treg ratios of 1:1 to 8:1 for 3 d. PI; proliferation index. “Tresp Only” (left) are Tresp cells cultured with anti-CD3. FIG. 27C shows qRT-PCR analysis of IL37, Foxp3, and Il17 expression in expanded WT and IL37Tg Treg cells+/1 anti-CD3/CD28 stimulated. Gapdh served as an internal control. Each symbol represents an individual mouse; small horizontal lines indicate mean±s.e.m. (n=1 mouse per group) (B), (n=3 mice per group) (FIG. 27C). Data represent mean±s.e.m. *p<0.05; **p<0.01; ***p<0.001. (Student's t-test). Data are representative of one (FIGS. 27A-B) or three (FIG. 27C) independent experiments.

FIGS. 28A-D show a series of graphs and microscopy images depicting the effects of ex vivo expanded IL37 mouse Treg cells suppressing contact hypersensitivity (CHS) in vivo. FIG. 28A shows a schematic of CHS experiments using the adoptive transfer of expanded Treg cells from WT or IL37 Tg mice (donor) to WT mice (recipient). FIG. 28B shows a line graph depicting ear swelling in mice adoptively transferred with PBS (DNFB+PBS), WT Treg cells (DNFB+WT-Treg) or IL37 Tg Treg cells (DNFB+IL37 Tg-Treg). FIG. 28C shows a series of H&E microscopic images of ear specimens taken at 48 h after DNFB challenge from mice adoptively transferred with PBS, WT-Treg, or IL37 Tg-Treg. Scale bar, 100 μm. FIG. 28D shows contour plots of CFSE-labeled cells among Treg cells (CD4+CD25^{hi}CD45RB^{lo}) and Tconv cells (CD4+CD25^{lo}CD45RB^{hi}) from ear specimens, taken at 48 h after DNFB challenge from mice adoptively transferred with WT-Treg or IL37 Tg Treg cells. Numbers in contour plots depict percentages of CFSE+ cells in Tconv or Treg cells. Data represent mean±s.e.m. *p<0.05. Data are representative of two experiments.

FIGS. 29A-D show a series of graphs and microscopy images depicting the effects of ex vivo expanded IL37 mouse Treg cells suppressing established inflammation in a mouse model of psoriasis. FIG. 29A shows a schematic of psoriasis experiments using the adoptive transfer of expanded Treg cells from WT or IL37 Tg mice (donor) to WT mice (recipient). FIG. 29B shows a line graph depicting ear swelling in mice stimulated with imiquimod every day and adoptively transferred with PBS (DNFB+PBS), WT Treg cells (DNFB+WT-Treg) or IL37 Tg Treg cells (DNFB+IL37 Tg-Treg) on D0. FIG. 29C shows a series of H&E microscopic images of ear specimens taken at D5 after adoptive transfer with PBS, WT-Treg, or IL37 Tg-Treg. Scale bar, 100 μm. FIG. 29D shows clinical images of mouse ears of WT mice that were adoptively transferred with no treatment (−CTL, −control), PBS (+CTL, +control), expanded WT Treg cells (+WT Treg) or IL37 Tg Treg cells (+IL37 Treg). Data represent mean±s.e.m. ****p<0.0001. Data are representative of two experiments.

FIGS. 30A-C show a series of graphs depicting the effects of ex vivo expanded IL37 mouse Treg cells suppressing neuroinflammation in a mouse model of traumatic brain injury (TBI). FIG. 30A shows a schematic of TBI experiments using the adoptive transfer of expanded Treg cells from WT or IL37 Tg mice (donor) to WT mice (recipient). FIG. 30B shows a line graph depicting neurological severity scores after TBI injury in mice adoptively transferred with PBS (DNFB+PBS), WT Treg cells (DNFB+WT-Treg) or IL37 Tg Treg cells (DNFB+IL37 Tg-Treg) 3 hours after TBI. FIGS. 30C-G show a series of graphs depicting the analysis of cervical lymph nodes of mice with TBI for CD3+ (19C), CD4+ (FIG. 30D), CD8+ (FIG. 30E), and Treg cell counts (FIG. 30F) from the cervical lymph nodes of mice with TBI. FIG. 30G shows histograms (left) and mean fluorescence intensity (MFI) quantification (right) on the surface of CD8+ T cells from the cervical lymph nodes. Each symbol represents an individual mouse; small horizontal lines indicate mean±s.e.m. (n=6 mice per group). Data represent mean±s.e.m. *p<0.05; **p<0.01; ***p<0.001. (Student's t-test). Data are representative of one independent experiment.

FIGS. 31A-C show a series of graphs depicting Treg cells and IL37-expressing cells in PBMC from healthy donors and type 1 diabetes (TID) patients using a flow cytometry-based PrimeFlow® RNA assay. FIG. 31A shows contour plots of CD25+CD127lo cell gating in human CD3+CD4+ T cells from healthy donors (left) and TID patients (middle), as well as the quantification of the percentage of CD3+CD4+CD25^{hi}CD127^{lo} cells (right). FIG. 31B shows bar graphs of CD3+, CD4+, CD8+, Treg, and natural killer (NK) cell numbers in PBMCs from healthy donors and TID patients. FIG. 31C shows histograms (left) and quantification by mean fluorescent intensity (MFI) (right) of Primeflow®, a flow cytometry-based assay, to measure IL37 mRNA expression in CD3+, CD4+, CD8+, Treg cells, and NK cells in PBMCs from healthy donors and TID patients. IgG was used for negative control staining (solid gray histogram). Each symbol represents an individual donor; small horizontal lines indicate mean±s.e.m. (n=5 human donors per group). *p<0.05; **p<0.01; ***p<0.001. (Student's t-test). Data are representative of two independent experiments.

FIG. 32 shows a series of bar graphs depicting gene expression analysis of ex vivo expanded human Treg cells. qRT-PCR analysis of IL37, FOXP3, IL17, STAT3, and GATA3 expression in T conventional (Tconv) cells, Treg cells without expansion (Treg Ctl), and expanded human Treg cells (Treg Blast). GAPDH served as an internal control. Each symbol represents an individual mouse; small horizontal lines indicate mean±s.e.m. (n=3 human donors per group). Data represent mean±s.e.m. *p<0.05; **p<0.01; ***p<0.001; ****p<0.0001. (Student's t-test). Data are representative of three independent experiments.

FIG. 33 shows a Vector map of IL37-BFP_Lenti.

FIGS. 34A-B show a series of graphs depicting gene expression changes of ex vivo expanded human Treg cells after IL37 transduction. FIG. 34A shows density plots of IL-37 expression from purified human Treg cells transduced with a control vector or IL37 overexpression (OE) vector (left) and quantification of the percent of IL-37+ Treg cells (far right). No stain used as a control. Right. FIG. 34B shows bar graphs of IL37, FOXP3, and Helios gene expression in human Treg cells transduced with a control vector or IL37 OE-vector. GAPDH served as an internal control. Each symbol represents an individual healthy donor; small horizontal lines indicate mean±s.e.m. (n=3 donors per group). Data represent mean±s.e.m. ***p<0.001; ****p<0.0001. (Student's t-test). Data are representative of three independent experiments.

FIGS. 35A-E show a series of graphs depicting that IL-37 overexpression into expanded human Treg cells sustains their suppressive function in a mouse model of graft-vs-host disease (GVHD). FIG. 35A shows a schematic depicting xenogeneic GVHD experiments using adoptive cell transfer of ex vivo expanded human Treg cells with control or IL37 OE vector to control xenogeneic GVHD in immunocompromised BRGS mice (recipient). FIG. 35B shows a line graph depicting clinical scores of BRGS mice over 34 days after injection with PBS, PBMC only, PBMC+control Treg cells (pbmc ctl), or PBMC+IL37 OE Treg cells (pbmc IL37). FIG. 35C shows bar graphs of CD45+ cell counts in the spleen on day 34. FIG. 35D shows dot blots of splenic CD4 and CD8 cells in mice that received PBMC and were treated with control Treg cells or IL37 OE Treg cells. FIG. 35E shows dot blots (left) and bar graphs (right) of splenic Treg cells in mice that received PBMC and were treated with control Treg cells or IL37 OE Treg cells. Data represent mean±s.e.m. *p<0.05; **p<0.01; (Student's t-test). Data are representative of 2 independent experiments.

FIGS. 36A-D show a series of graphs depicting that IL-37 overexpression into Jurkat cells induces Treg-like phenotype and function in a mouse model of xenogeneic graft-vs-host disease (GVHD). FIG. 36A shows a schematic depicting GVHD experiments using adoptive cell transfer of human PBMC with control or IL37 OE Jurkat cells (donor) to immunocompromised BRGS mice (recipient). FIG. 36B shows a line graph depicting clinical scores of BRGS mice over 12 days after injection with PBS, PBMC only, PBMC+control Jurkat cells, or PBMC+IL37 OE Jurkat cells (top), as well as clinical scoring criteria of GVHD used in the experiments (bottom). FIG. 36C shows images of colons (left) and spleens (right) from BRGS mice injected with PBMC only, PBMC+IL37 OE Jurkat cells, or PBMC+control Jurkat cells. FIG. 36D shows a bar graph depicting splenocyte counts from BRGS mice injected with PBS, PBMC only, PBMC+control Jurkat cells, or PBMC+IL37 OE Jurkat cells. Data represent mean±s.e.m. *p<0.05; **p<0.01; (Student's t-test). Data are representative of three independent experiments.

FIGS. 37A-E show a series of graphs depicting that the binding partner screening of FOXP3-regulating transcription factors identifies IL-37 as a key molecule to maintain FOXP3 in human Treg cells. FIG. 37A shows qRT-PCR analysis of proteins associated with the transcription factors: STAT5, NFAT, and Smad3 expression in purified human Tconv and Treg cells. GAPDH served as an internal control. FIG. 37B shows dot plots (left) and frequency (right) of FOXP3 expression among Tconv (CD25−) and Treg (CD25+) cells purified from human donors using a human CD4+CD25+CD127dim kit. Numbers in dot plots depict percentages of FOXP3+ cells in Tconv or Treg cells. FIG. 37C shows, in the top panel, contour plots of FOXP3+ cell gating in human CD4+ T cells transfected with scramble siRNA (Ctrl), siCHK1, siCREB1, siIL-37, siRPTOR, and siRUNX1 48 h prior, as well as in the bottom panel, quantification of the percentage of FOXP3+ T cells. Scramble Ctrl was used to measure *p-value. FIG. 37D shows Western blot analysis of protein expression levels in purified human CD4+ T cells 24 h after transfection with scrambled siRNA (Ctrl) or siRNA against CREB1, CHEK1 (pooled), IL37 (pooled), RPTOR (pooled), and RUNX1. Actin served as a loading control. FIG. 37E shows qRT-PCR analysis of FOXP3 gene expression following siRNA knockdown of IL37 in purified Treg cells. Top IL37 expression, bottom, FOXP3 expression; GAPDH served as an internal control. Each symbol represents an individual donor; small horizontal lines indicate mean±s.e.m., (n=4 donors per group) (A), (n=8 donors per group) (FIG. 37B), (n=5 donors per group) (FIG. 37C), (n=3 donors per group) (FIG. 37D, 37E). *p<0.05; **p<0.01; ***p<0.001; ****p<0.0001 (Student's t-test). Data are representative of two (FIG. 37E), three (FIG. 37C, 37D), four (FIG. 37A), or seven (FIG. 37B) independent experiments

### DETAILED DESCRIPTION

The present disclosure is based, at least in part, on the discovery that the nuclear expression of immunosuppressive cytokine IL-37 is required for the maintenance of FOXP3 expression in human regulatory T (Treg) cells and that IL-37 plays a critical role in the suppressive nature of these cells not only in a steady-state environment, but also in an inflammatory environment. FOXP3+ Treg cells play an essential role in the modulation of immune responses and maintenance of peripheral self-tolerance. Continual expression of FOXP3 in Treg cells is crucial in preserving tolerance and stabilizing Treg cell populations. Elevated nuclear IL-37 expression corresponds with elevated expression of FOXP3 and sustains FOXP3+ Treg cells during an inflammatory response resulting in increased suppression both in vitro and in vivo. The disclosure herein describes a method of producing a population of modified regulatory T cells or Treg-like cells that are capable of sustained suppressive function in vitro and in vivo even under inflammatory conditions. By avoiding conversion into conventional T (Tconv) cells (e.g. non-Treg CD4+ cells), the population of modified Treg cells of the present disclosure may provide improved clinical outcomes when used for adoptive therapy and for the treatment of an immune diseases or disorder (e.g., autoimmune disease and disorders).

Treg cells play a critical role in peripheral tolerance by suppressing and preventing an auto-reactive immune response. Understanding how Treg cells function in maintaining peripheral tolerance is key to preventing and treating autoimmunity and for the future development of immune-targeted therapies, such as adoptive Treg cell therapy. However, culturing and maintaining enough primary human Treg cells to perform molecular-based experiments is time-consuming and expensive. While novel genome editing techniques have improved the efficacy of modifying primary Treg cells, experimental challenges still exist due to many factors, including inter-personal and intra-personal heterogeneity of primary human Treg cells. The alternative to using primary Treg cells is to develop a Treg cell line easily usable for experiments. Multiple studies have attempted to identify or establish Treg-like cell lines by altering gene expression or inducing Treg cell differentiation in T cell lines but have yet to produce an artificial Treg cell line that is either stable or readily available for use.

The methods described herein take advantage of the suppressive properties of the modified CD4+ T cells disclosed herein and include methods of making and using a population of modified CD4+ T cells expressing nuclear IL-37 to treat an immune disease or disorder (e.g., autoimmune disease and disorders). In some embodiments, the method of treatment includes adoptive transfer using the population of modified CD4+ T cells. One of the significant challenges with the adoptive transfer of regulatory T cells in autoimmunity is that the regulatory T cells are unstable and can convert to inflammatory T cells exacerbating the disease. Disclosed herein are methods of inducing nuclear IL-37 expression in CD4+ T cells (e.g., Treg cells or Treg-like cells) and methods of using the same to treat an immune-disease or disorder (e.g., autoimmune disease and disorders). In some embodiments, the method of treatment includes adoptive transfer using the population of modified regulatory T cells. The methods disclosed herein will provide a way to minimize potential adverse effects of regulatory T cell therapy, such as, but not limited to, immunosuppression.

The methods of the present disclosure can generate antigen-specific regulatory T cells, wherein the T cells have increased specificity and increased potency in suppressing autoimmunity relative to an unmodified regulatory T cell. Antigen-specific Treg cells are challenging to develop, as antigens are not always known or uniform for each disease population. Using the methods disclosed herein, antigen-specific T effector cells can be converted from autoimmune patients to create antigen-specific Treg cells (e.g. modification of autologous T-cells). Thus, specific antigens do not have to be identified from each patient for successful therapy. The antigen-specific Treg cells would unlikely suppress other immune cells or other inflammatory conditions, minimizing potential side effects, such as non-specific immunosuppression.

Disclosed here are methods of making a using a population of modified regulatory T cells to treat an autoimmune disease or disorder comprising administering to a subject in need thereof a therapeutically effective amount of a composition comprising a population of modified regulatory T cells that express nuclear IL-37. In some embodiments, the methods disclosed herein utilize the patient's T effector cells. In some embodiments, the methods disclosed herein generate personalized Treg cells.

Provided herein are methods of producing a population of modified regulatory T cells comprising introducing into a plurality of human T cells a composition comprising an IL-37 or a nucleic acid sequence encoding the IL-37 under suitable conditions that express the IL-37 in the nucleus of the human T cell, thereby producing a plurality of modified regulatory T cells.

Also provided herein is a method of treating an immune disease or disorder comprising administering to a subject in need thereof a therapeutically effective amount of a composition comprising a population of modified CD4+ T cells expressing IL-37.

### CD4+ T Cells and Regulatory T Cells

“CD4+ T cells” is intended to have its standard definition as used in the art. In some embodiments, CD4+ T cells are CD3+CD4+CD8− T cells.

“Tconv cells” or “T conventional cells” as used herein are synonymous and are intended to have its standard definition as used in the art. In some embodiments, Tconv gells are non-Treg CD4+ T cells. Tconv cells include naïve CD4+ T cells (non-pathogenic).

“Teff cells” “CD4+ Teff cells” “effector T cells” or “Teffector cells” as used herein are synonymous and are intended to have its standard definition as used in the art. These are also also called T helper (Th) cells and are responsible for immune responses. In some embodiments, Teff cells include Th1, Th2, Th9, Th17, Th22, Thf cells or a combination thereof.

“Regulatory T lymphocyte” “T regulatory cell” “Regulatory T cell” or “Treg cell” or “Treg” as used in the present specification and claims are synonymous and are intended to have its standard definition as used in the art. Treg cells are a specialized subpopulation of T cells that act in a “regulatory” way to suppress activation of the immune system and thereby maintain immune system homeostasis and tolerance to self-antigens. Treg cells have sometimes been referred to suppressor T cells. Treg cells are characterized by the expression of the forkhead family transcription factor FOXP3 (forkhead box p3). They are characterized by the expression of CD4. They may express CD25 (also known as Interleukin 2 receptor subunit alpha, or IL2RA).

Treg cells have generally been identified as a CD4+CD25+ T cell population capable of suppressing an immune response. Identifying Foxp3 as a “master-regulator” of Treg cells helped define Treg cells as a distinct T cell lineage. The identification of additional antigenic markers on the surface of Treg cells has enabled the identification and FACS sorting of viable Treg cells to greater purity, resulting in a more highly-enriched and suppressive Treg population. In addition to CD4 and CD25, both mouse and human Treg cells express GITR/AITR, CTLA-4, and express low levels of CD127 (IL-7Ra). Exemplary Treg expression markers of include but are not limited to FOXP3, CD25, CD4, CTLA4, IL-10, GITR, TGF-beta and CD127.

Moreover, Treg cells can exist in different states, which can be identified based on their expression of surface markers. Treg cells that develop in the thymus from CD4+ thymocytes are known as “natural” Treg cells; however, Treg cells can also be induced in the periphery from naive CD4+ T cells in response to low-dose engagement of the TCR, TGF beta and IL-2. These “induced” Treg cells secrete the immunosuppressive cytokine IL-10. The phenotype of Treg cells changes again as they become activated, and markers including GARP in mice and humans, CD45RA in humans, and CD103 in mice have been shown to be useful for identifying activated Treg cells. Treg cells are important in maintaining immune cell homeostasis, as evidenced by undesirable consequences of genetic or physical ablation of the Treg population. Treg cells generally maintain order in the immune system by enforcing a dominant negative regulation on other immune cells. Broadly classified into natural or adaptive (induced) Treg cells; natural Treg cells are CD4+CD25+ T cells, which develop and emigrate from the thymus to play a role in immune homeostasis. Adaptive Treg cells are non-regulatory CD4+ T cells, which acquire CD25 (IL-2R alpha) expression outside the thymus, and may be induced by inflammation and disease processes, such as autoimmunity and cancer. Functional Treg cells can also be forced through the overexpression of either of the two common human FoxP3 isoforms in CD4+CD25− cells. These are classified as “forced Treg cells”. In some embodiments, “Treg-like cells” can be produced through the expression of nuclear IL-37 in CD4+ Tcells. Treg-like cells exhibit the properties of T regulatory cells, such as tolerance and stability. Treg like cells can play a role in modulation of immune responses and maintenance of self-tolerance.

In some embodiments, a modified CD4+ T cell expresses one or more marker selected from a group consisting of FOXP3, CD25, CD4, CTLA4, IL-10, GITR, TGF-β, and CD127. In some embodiments of the present disclosure, at least one cell surface marker is FOXP3 and CD25. In some embodiments, at least one cell surface marker is FOXP3.

In certain embodiments, the Treg cell is CD4+CD25+ and FOXP3+. In certain embodiments, the Treg cell is CD4+CD25+FOXP3+ and CD127low. In certain embodiments, the Treg cell is CD4+CD25+CD127low. In certain embodiments, the Treg cell is CD4+CD25+FoxP3+CD127low and CD45RA+.

In some embodiments, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the plurality of modified CD4+ T cells express at least one marker of a regulatory T cell. In some embodiments, at least about 75% of the plurality of modified CD4+ T cells express at least one marker of a regulatory T cell. In some embodiments, at least about 95% of the plurality of modified CD4+ T cells express at least one marker of a regulatory T cell.

The loss of FOXP3 under inflammatory conditions induces instability and plasticity of Treg cells, which raises considerable concerns in treating immune pathologies or antitumor immunity using Treg cells or T cells, respectively.

In some embodiments of the methods of the present disclosure, the population of modified CD4+ T cells are allogeneic regulatory T cells. In some embodiments, the population of modified CD4+ T cells are autologous regulatory T cells.

### Forkhead Box P3 (FOXP3)

Expression of the transcription factor forkhead box P3 (FOXP3) is required for the suppressive function of regulatory T cells. Mutation or deletion of FOXP3 leads to a loss of functional Treg cell populations and subsequent development of severe autoimmunity and/or inflammation in both humans and mice. Studies have shown that sustained FOXP3 expression and subsequent peripheral Treg (pTreg) cell stability are maintained by the transcriptional and epigenetic control of FOXP3 in its promoter and conserved non-coding DNA sequences.

As disclosed herein, human Treg cells express the highest IL-37 mRNA levels among blood cells isolated from healthy individuals. Knockdown of IL-37 expression in CD4+ T cells had a significant impact on FOXP3 expression (72% decrease in FOXP3 mRNA after IL-37 knockdown). Knockdown of FOXP3 expression did not significantly impact IL-37 expression. Therefore, there is evidence suggesting a critical role for IL-37 in regulating Treg cell function. Without wishing to be bound by theory, IL-37 may promote Treg suppressive function by its impact on FOXP3, and that high-level expression of IL-37 functions to promote and maintain human Treg cell stability.

In some embodiments, CD4+ T cells that are not Treg cells can be genetically engineered into Treg cells through the forced expression FOXP3. In some embodiments, FOXP3 can be encoded in a transgene with an inducible promoter, such that FOXP3 expression can be induced to create engineered Treg cells or “forced” Treg cells. In some embodiments, the FOXP3 is wild-type (WT) FOXP3.

In some embodiments of the methods of the present disclosure, the expression of FOXP3 in the plurality of modified CD4+ T cells is higher than the expression of FOXP3 in a population of wildtype human T cells. In some embodiments, the expression of FOXP3 in the plurality of modified CD4+ T cells is about 1-fold, about 2-fold, about 3-fold, about 4-fold, about 5-fold, about 6-fold, about 7-fold, about 8-fold, about 9-fold, or about 10-fold higher than the expression of FOXP3 in a population of wildtype human T cells.

In some embodiments, the expression of FOXP3 in the plurality of modified CD4+ T cells is at least about 5%, 10%, 15%, 20%, 25%, 30%, 35%, 40%, 45%, 50%, 60%, 65%, 70%, 75%, 80%, 85%, 90%, 95%, 100%, 200%, 300%, 400%, 500%, 600%, 700%, 800%, 900% or 1000% higher than the expression of FOXP3 in a population of wildtype human T cells.

In some embodiments, the increased expression of FOXP3 in the modified CD4+ T cells is dependent upon the introduction of a composition comprising an IL-37 or nucleic acid sequence encoding the IL-37. In some embodiments, the increased expression of FOXP3 in the population of modified CD4+ T cells maintains the immunosuppressive function of the modified CD4+ T cells in vitro and in vivo. In some embodiments, the increased expression of FOXP3 in the population of modified CD4+ T cells prevents the conversion of the modified CD4+ T cells to inflammatory T cells in vitro and in vivo. In some embodiments, the increased expression of FOXP3 in the population of modified CD4+ T cells prolongs the immunosuppressive function of the modified CD4+ T cells in vitro and in vivo.

### Interleukin-37

Interleukin-37 (IL-37) is a unique dual-function cytokine that functions intracellularly and extracellularly. IL-37 is one of eleven IL-1 family members and the only known member to be broadly anti-inflammatory. IL-37 is transcribed as five different splice variants (IL-37a-e) and is expressed in human cells but not mouse cells. Yet similarly to other IL-1 family members, IL-37 has no species specificity and exhibits effects on murine cells that are comparable to those on human cells.

IL-37 is an anti-inflammatory cytokine that participates in immune tolerance by generating semi-mature tolerogenic dendritic cells (DCs) in antigen-specific adaptive immune responses. Human regulatory T cells express the highest IL-37 levels among all T-cell subsets. Since T cells do not secrete IL-37, IL-37 may play an intracellular role.

A plurality of modified CD4+ T cells of the population produced by the method of the present disclosure comprises an Interleukin-37 (IL-37) or a nucleic acid sequence encoding the IL-37 under suitable conditions that express the IL-37 in the nucleus of the T cell, wherein at least about 2%, 5%, 10%, 15%, 20%, 25%, 30%, 35%, 40%, 45%, 50%, 60%, 65%, 70%, 75%, 80%, 85%, 90%, 95%, 99%, 100% or any percentage in between of the plurality of cells of the population comprise the nuclear IL-37 or the nucleic acid sequence encoding the IL-37. In some embodiments, about 2%, 5%, 10%, 15%, 20%, 25%, 30%, 35%, 40%, 45%, 50%, 60%, 65%, 70%, 75%, 80%, 85%, 90%, 95%, 99%, 100% or any percentage in between of the plurality of modified T cells of the population further expresses one or more marker(s) of a regulatory T (Treg) cell.

In some embodiments, the nuclear expression of IL-37 in the plurality of modified CD4+ T cells is at least about 1-fold, at least about 2-fold, at least about 3-fold, at least about 4-fold, at least about 5-fold, at least about 6-fold, at least about 7-fold, at least about 8-fold, at least about 9-fold or at least about 10-fold greater than the nuclear expression of IL-37 in a population of wildtype human T cells. In some embodiments, the nuclear expression of IL-37 in the plurality of modified CD4+ T cells is at least about 5-fold greater than the nuclear expression of IL-37 in a population of wildtype human T cells.

In some embodiments, the nuclear expression of IL-37 in the plurality of modified CD4+ T cells is about 1-fold to 2-fold, about 1-fold to 3-fold, about 1-fold to 4-fold, about 1-fold to 5-fold, about 1-fold to 6-fold, about 1-fold to 7-fold, about 1-fold to 8-fold, about 1-fold to 9-fold, about 1-fold to 10-fold, about 2-fold to 3-fold, about 2-fold to 4-fold, about 2-fold to 5-fold, about 2-fold to 6-fold, about 2-fold to 7-fold, about 2-fold to 8-fold, about 2-fold to 9-fold, about 2-fold to 10-fold, about 3-fold to 4-fold, about 3-fold to 5-fold, about 3-fold to 6-fold, about 3-fold to 7-fold, about 3-fold to 8-fold, about 3-fold to 9-fold, about 3-fold to 10-fold, about 4-fold to 5-fold, about 4-fold to 6-fold, about 4-fold to 7-fold, about 4-fold to 8-fold, about 4-fold to 9-fold, about 4-fold to 10-fold, about 5-fold to 6-fold, about 5-fold to 7-fold, about 5-fold to 8-fold, about 5-fold to 9-fold, about 5-fold to 10-fold, about 6-fold to 7-fold, about 6-fold to 8-fold, about 6-fold to 9-fold, about 6-fold to 10-fold, about 7-fold to 8-fold, about 7-fold to 9-fold, about 7-fold to 10-fold, about 8-fold to 9-fold, about 8-fold to 10-fold, about 9-fold to 10-fold greater than the nuclear expression of IL-37 in a population of wildtype human T cells. In some embodiments, the nuclear expression of IL-37 in the plurality of modified CD4+ T cells is about 5-fold to about 10-fold greater than the nuclear expression of IL-37 in a population of wildtype human T cells.

In some embodiments, the nuclear expression of IL-37 in the plurality of modified CD4+ T cells is at least about 5%, 10%, 15%, 20%, 25%, 30%, 35%, 40%, 45%, 50%, 60%, 65%, 70%, 75%, 80%, 85%, 90%, 95%, 100%, 200%, 300%, 400%, 500%, 600%, 700%, 800%, 900% or 1000%, or any percent in between, greater than the nuclear expression of IL-37 in a population of wildtype human T cells.

In some embodiments, the expression of nuclear IL-37 in the population of modified CD4+ T cells is at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95% or at least about 100% greater than the expression of nuclear IL-37 in a population of wildtype CD4+ T cells. In some embodiments, the expression of nuclear IL-37 in the population of modified CD4+ T cells is at least about 50% greater than the expression of nuclear IL-37 in a population of wildtype CD4+ T cells. In some embodiments, the expression of nuclear IL-37 in the population of modified CD4+ T cells is at least about 85% greater than the expression of nuclear IL-37 in a population of wildtype CD4+ T cells.

In some embodiments, the expression of nuclear IL-37 in the population of modified CD4+ T cells is about 10% to 20%, about 10% to 30%, about 10% to 40%, about 10% to 50%, about 10% to 60%, about 10% to 70%, about 10% to 80%, about 10% to 90%, about 10% to 100%, about 20% to 30%, about 20% to 40%, about 20% to 50%, about 20% to 60%, about 20% to 70%, about 20% to 80%, about 20% to 90%, about 20% to 100%, about 30% to 40%, about 30% to 50%, about 30% to 60%, about 30% to 70%, about 30% to 80%, about 30% to 90%, about 30% to 100%, about 40% to 50%, about 40% to 60%, about 40% to 70%, about 40% to 80%, about 40% to 90%, about 40% to 100%, about 50% to 60%, about 50% to 70%, about 50% to 80%, about 50% to 90%, about 50% to 100%, about 60% to 70%, about 60% to 80%, about 60% to 90%, about 60% to 100%, about 70% to 80%, about 70% to 90%, about 70% to 100%, about 80% to 90%, about 80% to 100%, about 90% to 100% greater than the expression of nuclear IL-37 in a population of wildtype CD4+ T cells. In some embodiments, the expression of nuclear IL-37 in the population of modified CD4+ T cells is about 50% to about 80% greater than the expression of nuclear IL-37 in a population of wildtype CD4+ T cells.

In some embodiments of the methods of the present disclosure, the increased nuclear expression of IL-37 in the population of modified CD4+ T cells causes increased expression of FOXP3 in the same population. In some embodiments, the higher expression of nuclear IL-37 in the population of modified CD4+ T cells compared to IL-37 expression in a population of wildtype human T cells causes a higher expression of FOXP3 in the modified CD4+ T cells compared to FOXP3 expression in the population of wildtype human T cells. In some embodiments, the increased expression of nuclear IL-37 in the population of modified CD4+ T cells maintains the immunosuppressive function of the modified CD4+ T cells in vitro and in vivo. In some embodiments, the increased expression of nuclear IL-37 in the population of modified CD4+ T cells prevents the conversion of the modified CD4+ T cells to inflammatory T cells in vitro and in vivo. In some embodiments, the increased expression of nuclear IL-37 in the population of modified CD4+ T cells prolongs the immunosuppressive function of the modified CD4+ T cells in vitro and in vivo. In some embodiments, the increased expression of nuclear IL-37 in the population of modified CD4+ T cells maintains the stability of an immunosuppressive phenotype of the modified CD4+ T cells in vitro and in vivo. In some embodiments, the expression of nuclear IL-37 in the modified CD4+ T cells prevents the loss of FOXP3 expression in the modified CD4+ T cells under inflammatory or pathogenic circumstances compared to the loss of FOXP3 expression in wildtype human T cells under similar conditions in vitro and in vivo.

Among all IL-1 family members, IL-37 is unique because of its broad suppression in innate and adaptive immunity. Since IL-37 is such a potent anti-inflammatory/anti-immune cytokine, the synthesis, activation, and secretion of IL-37 is tightly controlled. In humans, IL37 mRNA and protein levels are usually low.

The present disclosure provides methods of producing a population of modified CD4+ T cells comprising introducing into a plurality of human T cells, a composition comprising an IL-37 or a nucleic acid sequence encoding the IL-37 under suitable conditions that express the IL-37, thereby producing a plurality of modified CD4+ T cells. In some embodiments of the methods of the present disclosure, the plurality of modified CD4+ T cells express IL-37 in the nucleus and the cytoplasm of the cells. In some embodiments, the modified CD4+ T cells express IL-37 in the nucleus. In some embodiments, the levels of IL-37 in the nucleus are higher than IL-37 levels in the cytoplasm. In some embodiments, the levels of IL-37 in the nucleus and the cytoplasm are the same. In some embodiments, the levels of IL-37 is lower in the nucleus than in the cytoplasm. In some embodiments, introducing into a plurality of human T cells a composition comprising an IL-37 or a nucleic acid sequence encoding the IL-37 increases the levels of IL-37 in the nucleus of the modified T cells.

In some embodiments, the ratio of nuclear IL-37 to cytoplasmic IL-37 in the plurality of modified CD4+ T cells is about 1:1, 2:1, 3:1, 4:1, 5:1, 6:1, 7:1, 8:1, 9:1 or 10:1. In some embodiments, the ratio of nuclear IL-37 to cytoplasmic IL-37 in the plurality fo modified CD4+ T cells is about 0:10, 1:9; 2:8, 3:7, 4:6, 5:5; 6:4, 7:3, 8:2, 9:1 or 10:0. In some embodiments, the ratio of nuclear IL-37 to cytoplasmic IL-37 in the plurality fo modified CD4+ T cells is about 4:6, 5:5; 6:4, 7:3 or 8:2. In some embodiments, the ratio of nuclear IL-37 to cytoplasmic IL-37 in the plurality of modified CD4+ T cells is about 4:6.

In some embodiments, the population of modified CD4+ T cells comprises non-Treg CD4+ T cell and the ratio of nuclear IL-37 to cytoplasmic IL-37 is about 0:100. In some embodiments, the population of modified CD4+ T cells comprises Treg cells and the ratio of nuclear to cytoplasmic IL-37 is about 5:5. In some embodiments, the population of modified CD4+ T cells comprises Treg cells and the ratio of nuclear to cytoplasmic IL-37 is about 4:6. In some embodiments, the population of modified CD4+ T cells comprises Treg cells and the ratio of nuclear to cytoplasmic IL-37 is about 6:4

Like macrophages and monocytes, T cells have been proven to express IL-37. However, different from macrophages and monocytes, T cells do not secrete IL-37, implicating an intracellular role for IL-37 in T cells. The first data came from the study of human Treg cells, where the cytoplasmic expression of IL-37 in human Treg cells led to Treg signature expressions such as secretion of TGF-β and IL-10 and expression of CTLA-4 and FOXP3, resulting in their immunosuppressive function. IL-37 expression has been detected in T cells from IL37 transgenic (Tg) mice and adoptive transfer of IL-37-expressing CD3+ T cells attenuated the DSS-induced chronic inflammatory bowel disease in mice.

IL-37 mRNA and protein levels are usually low and induced by stimulation with pro-inflammatory cytokines in macrophages; however, this does not hold in Treg cells. Compared to monocytes, IL-37 mRNA levels are high in human Treg cells without stimulation (over 10 times).

Recently, human Treg cells have been shown to express the highest IL37 mRNA levels among blood cells isolated from healthy individuals, and its expression is much higher in melanoma patients, suggesting that Treg cells with high IL-37 expression are highly suppressive and could, therefore, dampen antitumor immunity. Furthermore, a novel role for IL-37 in regulating FOXP3 expression and maintaining human Treg cell stability has recently been uncovered. Loss of FOXP3 under inflammatory conditions induces instability and plasticity of Treg cells, which raises considerable concerns in treating immune pathologies or antitumor immunity using Treg cells or T cells, respectively.

The present disclosure provides methods of producing a population of modified CD4+ T cells comprising introducing into a plurality of human T cells, a composition comprising an IL-37 or a nucleic acid sequence encoding the IL-37 under suitable conditions that express the IL-37 in the human T cell, thereby producing a plurality of modified regulatory T cells, wherein the IL-37 regulates FOXP3 expression in the modified T cells. In some embodiments, the increased IL-37 maintains FOXP3 levels in the modified CD4+ T cells under inflammatory conditions. In some embodiments, the FOXP3 levels in modified CD4+ T cells are maintained at higher levels compared to wildtype human T cells in inflammatory conditions. In some embodiments, the increased IL-37 in the modified CD4+ T cells maintains stability of the immunosuppressive phenotype of the modified T cells under steady-state or under inflammatory conditions.

Dysregulated IL-37 expression has been reported in autoimmune diseases (RA, SLE, Hashimoto thyroiditis, inflammatory bowel disease, Graves' disease, multiple sclerosis), ankylosing spondylitis, asthma, cardiovascular diseases, cerebral ischemia, hepatic disorders, infections, type 2 diabetes, and cancers. Downregulated IL-37 levels in active psoriasis skin were normalized when the disease was controlled with a JAK inhibitor tofacitinib. Similarly, downregulated IL-37 levels in active atopic dermatitis skin were reversed after treatment with the JAK/SYK inhibitor, ASN002. These data show a clear correlation of IL-37 expression with T-cell-mediated skin inflammation (Th17 in psoriasis and Th2 in atopic dermatitis), suggesting an immunosuppressive role for IL-37 in human diseases.

The present disclosure provides methods of treating an autoimmune disease or disorder comprising administering to a subject in need thereof a therapeutically effective amount of a composition comprising a population of modified CD4+ T cells that express IL-37. In some embodiments, the modified CD4+ T cells express IL-37 in the nucleus and the cytoplasm. In some embodiments, the modified CD4+ T cells express IL-37 in the nucleus. In some embodiments, the modified CD4+ T cells express higher levels of IL-37 in the nucleus than in the cytoplasm. In some embodiments, the modified CD4+ T cells express equal levels of IL-37 in the nucleus as in the cytoplasm. In some embodiments, the modified CD4+ T cells express lower levels of IL-37 in the nucleus than in the cytoplasm.

### Exemplary Modified Regulatory T-Cells (Treg-Like Cell Lines)

The Jurkat cell line is an immortalized T lymphocyte cell line that was initially obtained from the peripheral blood of a 14-year-old male patient with T cell leukemia. Jurkat, Clone E6-1 is a clone of the Jurkat-FHCRC cell line, a derivative of the Jurkat cell line, which was established from the patient's peripheral blood described above. This cell line can be used in immune system disorder research and immunology and immuno-oncology research. The Jurkat cell line has most often been used as a prototypical T cell line to study multiple events in T cell biology.

Developing a Treg-like cell line requires stable expression of the transcription factor forkhead box P3 (FOXP3), a key factor used to identify Treg cells and required for their suppressive capabilities. A 2007 study transfected the non-Treg CD4 T cell leukemia cell line, E6 Jurkat cells, with a FOXP3 overexpression vector to create a Treg-like cell line (Kim et. al., Functional and genomic analyses of FOXP 3-transduce Jurkat-T cells as regulatory T (Treg)-like cells, in the journal of Biochem Biophys Res. Commun). This study found that overexpression of FOXP3 in Jurkat cells led to an elevation in surface expression of cytotoxic T-lymphocyte antigen 4 (CTLA-4) and CD25. While these cells suppressed the proliferation of conventional T (Tconv) cells, the suppressive activity was significantly lower compared to primary Treg cells, suggesting that alternative molecules are required to create functionally comparable artificial Treg cells from Jurkat cells.

Recently, the anti-inflammatory IL-1 cytokine family member IL-37 was shown to contribute to the expression of FOXP3. Knockdown of IL37 in primary human Treg cells resulted in a significant decrease in FOXP3 and promoted T cell proliferation and differentiation. Treatment of human Treg cells with recombinant IL-37 also upregulated FOXP3 and enhanced the suppressive activity. These studies indicate that IL-37 expression in human Treg cells contributes to FOXP3 expression and Treg cell function, prompting us to hypothesize overexpressing IL-37 in non-Treg CD4 T cells such as Jurkat cells would result in the generation of human Treg-like cells.

However, it has been unclear whether T cells express IL-37. It has been reported that monocytes and dendritic cells are the main producers of IL-37 in human peripheral blood mononuclear cells (PBMCs) from healthy donors and only 0.5% of T cells expressed IL-37. On the other hand, another study showed that IL37 mRNA and IL-37 protein were highly expressed in human T cells, including Treg cells from healthy volunteers. Indeed, about 70% of Treg cells expressed IL-37 protein, indicating that Treg cells are one of the significant contributors to IL-37 expression.

In some embodiments, the population of modified CD4+ T cells are derived from a human T-cell. In some embodiments, the human T-cell is derived from a primary human T-cell population. In some embodiments, the human T-cell is derived from a T-cell line. In some embodiments, the T-cell line is a Jurkat T-cell line.

### Compositions and Uses of Modified CD4+ T Cells

In some embodiments, the method of the present disclosure produces a composition comprising a population of modified CD4+ T cells. In some embodiments, the method of the present disclosure produces a composition comprising a population of modified T regulatory cells or Treg-like cells.

In some embodiments of the methods of the disclosure, a buffer comprises the plurality of modified regulatory T cells or precursors thereof. The buffer maintains or enhances a level of cell viability and/or a Treg phenotype of the immune cell or precursor thereof, including T cells. In certain embodiments, the buffer maintains or enhances a level of cell viability and/or a Treg phenotype of the primary human T cells prior to the nucleofection. In certain embodiments, the buffer maintains or enhances a level of cell viability and/or a Treg phenotype of the primary human T cells during the nucleofection. In certain embodiments, the buffer maintains or enhances a level of cell viability and/or a Treg phenotype of the primary human T cells following the nucleofection. In certain embodiments, the buffer comprises one or more of KCl, MgCl2, ClNa, Glucose and Ca(NO₃)₂ in any absolute or relative abundance or concentration, and, optionally, the buffer further comprises a supplement selected from the group consisting of HEPES, Tris/HCl, and a phosphate buffer. In certain embodiments, the buffer comprises 5 mM KCl, 15 mM MgCl2, 90 mM ClNa, 10 mM Glucose and 0.4 mM Ca(NO₃)₂. In certain embodiments, the buffer comprises 5 mM KCl, 15 mM MgCl2, 90 mM ClNa, 10 mM Glucose and 0.4 mM Ca(NO₃)₂ and a supplement comprising 20 mM HEPES and 75 mM Tris/HCl. In certain embodiments, the buffer comprises 5 mM KCl, 15 mM MgCl2, 90 mM ClNa, 10 mM Glucose and 0.4 mM Ca(NO3)2 and a supplement comprising 40 mM Na2HPO4/NaH2PO4 at pH 7.2. In certain embodiments, the composition comprising modified regulatory T cells or precursors thereof comprises 100 μl of the buffer and between 5×10⁶ and 25×10⁶ cells. In certain embodiments, the composition comprises a scalable ratio of 250×10⁶ human T cells per milliliter of buffer or other media during the introduction step.

In some embodiments of the methods of the disclosure, the methods comprise contacting an immune cell of the disclosure, including a regulatory T cell of the disclosure, and a T cell expansion composition. In some embodiments of the methods of the disclosure, the step of introducing an IL-37 or a nucleic acid sequence encoding an IL-37 of the disclosure into an immune cell of the disclosure may further comprise contacting the immune cell and a T cell expansion composition. In some embodiments, including those in which the introducing step of the methods comprises an electroporation or a nucleofection step, the electroporation or a nucleofection step may be performed with the immune cell contacting T cell expansion composition of the disclosure.

### Methods of Expressing an IL-37

The disclosure provides methods of expressing an IL-37 in a modified CD4+ T cell. The method comprises (a) obtaining a cell population; (b) contacting the cell population to a composition comprising an IL-37 or a nucleic acid sequence encoding the IL-37 under conditions sufficient to transfer the IL-37 or nucleic acid encoding the IL-37 across a cell membrane of at least one cell in the cell population, thereby generating a modified cell population; (c) culturing the modified cell population under conditions suitable expression of the IL-37 or nucleic acid sequence encoding the IL-37 in the nucleus of the T cell; and (d) expanding and/or selecting at least one cell from the modified cell population that express the IL-37.

In some aspects, the cell population can comprise a plurality of modified human CD4+ T cells that express at least one marker of a regulatory T cell, wherein at least one marker of a regulatory T cell is selected from a group consisting of CD4, FOXP3, CD25, CD4, CTLA4, IL-10, GITR, TGF-β, and CD127. In some embodiments, at least one marker is FOXP3. In some embodiments, at least one marker is FOXP3 and CD25. The cell population can comprise FOXP3+ and FOXP3+CD25+ regulatory T cells in an optimized ratio.

In some aspects, the conditions sufficient to transfer the IL-37 or the nucleic acid sequence encoding the IL-37 across a cell membrane of at least one cell in the cell population comprises at least one application of one or more pulses of electricity at a specified voltage, a buffer, and one or more supplemental factor(s). In some aspects, the conditions suitable for integration of the sequence encoding the IL-37 comprise at least one of a buffer and one or more supplemental factor(s).

The expansion and selection steps can occur concurrently or sequentially. The expansion can occur prior to selection. The expansion can occur following selection, and, optionally, a further (i.e. second) selection can occur following expansion. Concurrent expansion and selection can be simultaneous. The expansion and/or selection steps can proceed for a period of 1 to 7 days, inclusive of the endpoints.

In some aspects, wherein the IL-37 or nucleic acid encoding an IL-37 comprises a selection gene, the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

The disclosure provides a composition comprising the population of a plurality of modified, expanded and selected CD4+ human T cell population of the methods described herein.

### Vector Compositions

The present disclosure provides compositions and methods for delivering an IL-37 or a nucleic acid sequence encoding an IL-37 to a regulatory T cell or a population of regulatory T cells. Non-limiting examples of compositions for delivery of a composition of the disclosure to a cell or a population of cells include a transposon or a vector. Thus, the present disclosure provides a vector comprising a nucleic acid sequence encoding an IL-37.

A vector comprising an IL-37 of the disclosure can further comprise a selection gene. The selection gene can encode a gene product essential for cell viability and survival. The selection gene can encode a gene product essential for cell viability and survival when challenged by selective cell culture conditions. Selective cell culture conditions may comprise a compound harmful to cell viability or survival and wherein the gene product confers resistance to the compound. Non-limiting examples of selection genes include neo (conferring resistance to neomycin), DHFR (encoding Dihydrofolate Reductase and conferring resistance to Methotrexate), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCE, RAD51C (encoding RAD51 Paralog C), GCS (encoding glucosylceramide synthase), NKX2.2 (encoding NK2 Homeobox 2), or any combination thereof.

### Methods of Treatment

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutically effective amount of the composition or pharmaceutical composition. In one aspect, the subject is a mammal. Preferably, the subject is human. The terms “subject” and “patient” are used interchangeably herein.

The present disclosure provides a method of treating an immune disease or disorder (e.g. autoimmune disease or disorder) or an inflammatory disease or disorder comprising administering to a subject in need thereof a therapeutically effective amount of a composition comprising a population of modified regulatory T cells that express IL-37. Immune disease or disorders include conditions that result from abnormal activity of the immune cells, overreacting to or attacking the body, displaying an extreme inflammatory response or loss of the ability to recognize and fight against non-self cells. Exemplary immune diseases or disorders include but are not limited to 1) allergies such as allergic contact hypersensitivity; 2) alloreactivity such as graft versus host disease and transplant rejection; and 3) autoimmune diseases or disorders such as type 1 diabetes, systemic lupus erythematosus, inflammatory bowel disease, Crohn's disease, ulcerative colitis and multiple sclerosis. In some embodiments, the immune disease or disorder is allergic contact hypersensitivity. In some embodiments, the immune disease or disorder is graft versus host disease. In some embodiments, the immune disease or disorder is inflammatory bowel disease. In some embodiments, the immune disease or disorder is type 1 diabetes.

Exemplary inflammatory disease or disorders include but are not limited to conditions affecting the digestive system, joints, skin, respiratory system or nervous system. In some embodiments, the inflammatory disease or disorder is selected from the group consisting of psoriasis, traumatic brain injury, bronchitis and pneumonitis. In some embodiments, the inflammatory disease or disorder is psoriasis. In some embodiments, the inflammatory disease or disorder is traumatic brain injury. In some embodiments, the inflammatory disease or disorder is bronchitis. In some embodiments, the inflammatory disease or disorder is and pneumonitis.

In some embodiments, the population of modified CD4+ T cells are allogeneic regulatory T cells. In some embodiments, the population of modified CD4+ T cells are autologous regulatory T cells.

In preferred aspects, the treatment of an immune disorder or disease comprises adoptive cell therapy. For example, in one aspect, the disclosure provides modified CD4+ T cells that express IL-37 and at least one marker of a regulatory T cell wherein the modified regulatory T cells have been selected and/or expanded for administration to a subject in need thereof. Modified cells can be formulated for storage at any temperature including room temperature and body temperature. Modified cells can be formulated for cryopreservation and subsequent thawing. Modified cells can be formulated in a pharmaceutically acceptable carrier for direct administration to a subject from sterile packaging. Modified cells can be formulated in a pharmaceutically acceptable carrier with an indicator of cell viability and/or IL-37 expression level to ensure a minimal level of cell function and IL-37 expression. Modified cells can be formulated in a pharmaceutically acceptable carrier at a prescribed density with one or more reagents to inhibit further expansion and/or prevent cell death.

The method of treatment can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, a mitotic inhibitor, a radiopharmaceutical).

In some aspects, the subject does not develop graft vs. host (GvHD) and/or host vs. graft (HvGD) following administration. In one aspect, the administration is systemic. Systemic administration can be any means known in the art and described in detail herein. Preferably, systemic administration is by intravenous injection or an intravenous infusion. In one aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously.

In some aspects, where the composition is autologous cells or allogeneic cells, the dose is an amount sufficient for the cells to engraft and/or persist for a sufficient time to treat the disease or disorder.

In some aspects of the methods of treatment described herein, the treatment can be modified or terminated. Treatment may be modified or terminated in response to, for example, a sign of recovery or a sign of decreasing disease severity/progression, a sign of disease remission/cessation, and/or the occurrence of an adverse event.

### Formulations, Dosages and Modes of Administration

The present disclosure provides formulations, dosages and methods for administration of the compositions of modified CD4+ T cells described herein.

The disclosed compositions can further comprise at least one of any suitable auxiliary, such as but not limited to, diluent, binder, stabilizer, buffers, salts, lipophilic solvents, preservative, adjuvant or the like. Pharmaceutically acceptable auxiliaries are preferred. Non-limiting examples of and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

### Nucleic Acid Molecules

Nucleic acid molecules of the disclosure encoding an IL-37 can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise nucleotide sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art.

The isolated nucleic acid compositions of this disclosure, such as RNA, cDNA, genomic DNA, or any combination thereof, can be obtained from biological sources using any number of cloning methodologies known to those of skill in the art. Methods of amplification of RNA or DNA are well known in the art and can be used according to the disclosure without undue experimentation, based on the teaching and guidance presented herein.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. The term “introducing” is intended to mean presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

### Example 1: Expression of IL-37 Induces a Regulatory T Cell-Like Phenotype and Function in Jurkat Cells

The present Example describes the materials and methods used in Examples 1-7.

### Materials and Methods

### Cell Preparation and Culture

Human leukemia cell line E6 Jurkat cells were obtained from ATCC (Manassas, VA) (clone E6-1). Cells were cultured in RPMI 1640 with L-glutamine (Gibco, Thermo Fisher) and supplemented with 10% FBS (Gemini BioProducts), 10 nM HEPES (Life Technologies, Thermo Fisher), 1% non-essential amino acids (Life Technologies), and 1 mM sodium pyruvate (Life Technologies, Thermo Fisher) in a humified incubator at 37° C. and 5% CO₂. Cells were cultured at 2×10⁵/ml in a Nunc™ EasYFlask T75, passaged when cell counts reached 1×10⁶/ml, and reseeded at 1:5 dilution in a new flask.

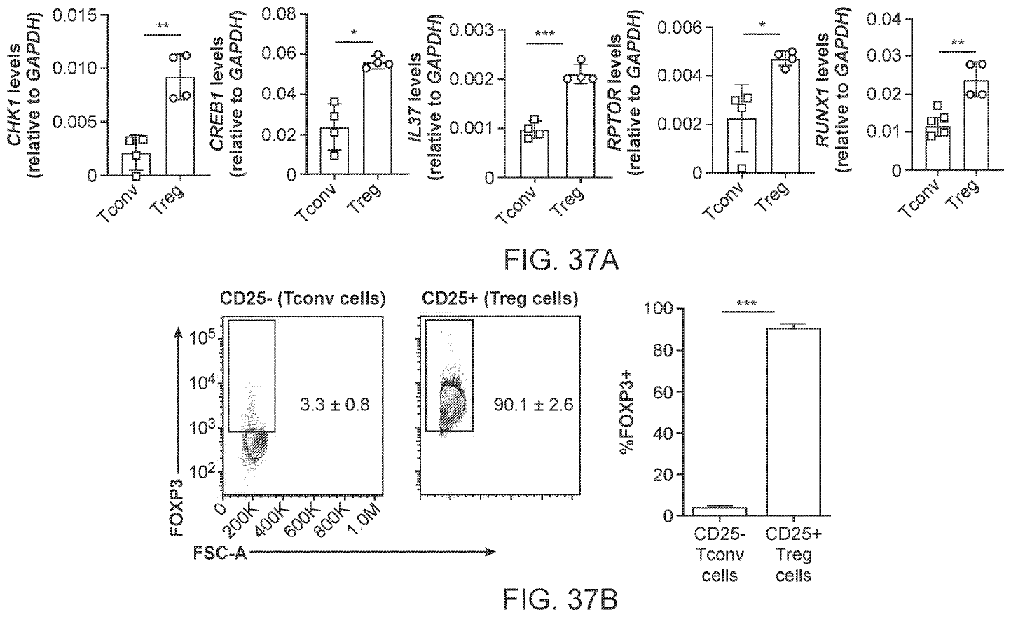

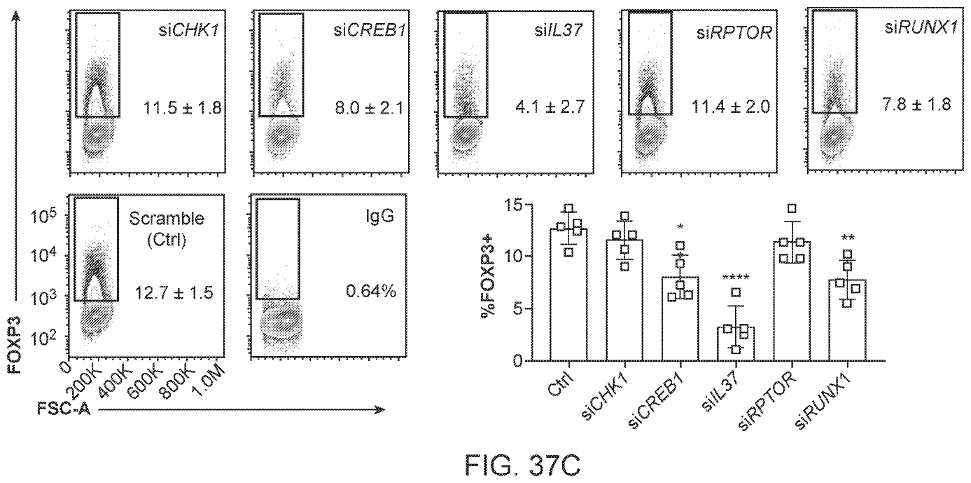

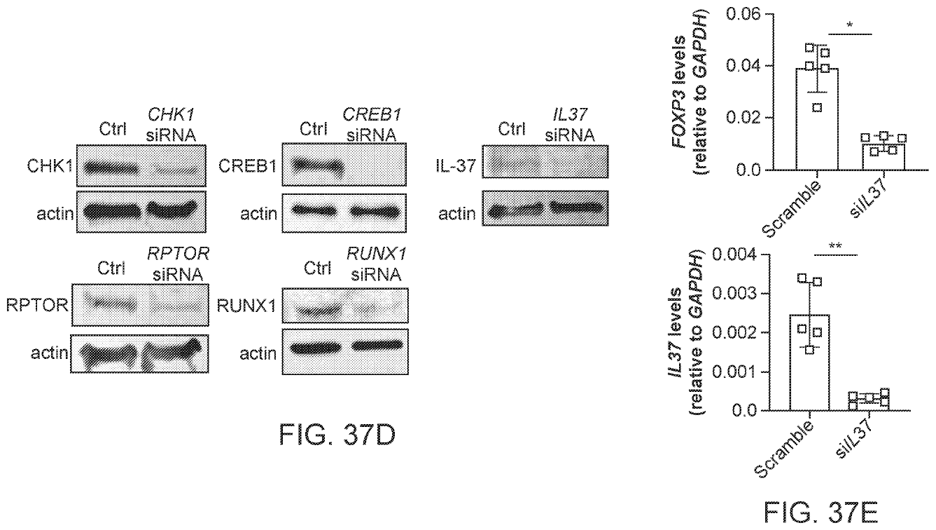

Human peripheral blood from healthy donors was obtained from blood cones provided by Children's Hospital Blood Donor Center (Aurora, CO) and peripheral blood mononuclear cells (PBMC) were purified using Ficoll density gradient centrifugation. Human primary T cells were isolated from blood samples were obtained from healthy human donors. After Ficoll density gradient centrifugation, cells were purified into CD4+ T cells, CD4+CD25−CD127^{hi} (Tconv) cells, and CD4+CD25+CD 127^{dim} (Treg) cells using a Human CD4+ T cell Isolation Kit (Miltenyi Biotech, Auburn, CA) and the Human CD4+CD25+CD127^{dim} Regulatory T cell Isolation Kit (Miltenyi Biotech) according to manufacturer's instructions. Purity of human Treg cells was confirmed to be 94-96% by flow cytometry using CD4, CD25 and FOXP3 surface markers. Purification was repeated twice to minimize the contamination of Tconv cells. FOXP3+ Treg cell purity check following human cell isolation is shown in FIG. 37B. All use of human blood specimens at the University of Colorado AMC was approved by the Colorado Institutional Review Board.

Mouse CD4+ T cells and CD4+CD25+Treg cells were purified from the splenocytes of C57BL/6, IL37 Tg or D20A mice using the Mouse CD4+ T cell Isolation Kit (Miltenyi Biotech) and the Mouse CD4+CD25+Regulatory T cell Isolation Kit (Miltenyi Biotech) according to manufacturer's instructions. For purification of either mouse or human Treg cells, MS column purification steps were performed in duplicate to ensure high purity of CD4+CD25+ T cells. Foxp3+ Treg cell purity check following mouse cell isolation. Mouse and human CD4+CD25-Tconv/Tresp cells were acquired as the dump population of CD4+ T cell remaining after CD25+Treg cell purification.

The caspase-1 inhibitor (Ac-YVAD-CMK) was purchased from Sigma-Aldrich. PAK1/2/3 inhibitor FRAX597 was purchased from Tocris. Flagellin was purchased from Invivogen.

### Mice

IL37 Tg mice have been described previously (Garlanda et. al., 2004; Nold et. al., 2010). The Dinarello lab produced the IL37^{D20A}Tg (D20A) mice described herein, and further described in Suzhao et. al. (S. Li et. al., 2019). C57BL/6 (WT) mice were purchased from Taconic Biosciences. The presence of the transgenes encoding IL37 and D20A was confirmed by genotyping PCR (Transnetyx) using tail clips obtained from mice 3-4 weeks of age. Only mice between the ages of 6 and 12 weeks were used for the experiments. All mice were maintained in a specific pathogen-free animal facility at the University of Colorado AMC and handled in accordance with the institutional guidelines. All mouse experiments were approved by the Institutional Animal Care and Use Committee of University of Colorado Denver.

Mouse CD4+T cells and CD4+CD25+ Treg cells were purified from the splenocytes of C57BL/6, IL37 transgenic (Tg) or D20A mice using the Mouse CD4+ T cell Isolation Kit (Miltenyi Biotech) and the Mouse CD4+CD25+ Regulatory T cell Isolation Kit (Miltenyi Biotech) according to manufacturer's instructions. For purification of mouse Treg cells, MS column purification steps were performed in duplicate to ensure high purity of CD4+CD25+ T cells. Foxp3+ Treg cell purity check following mouse cell isolation was performed. Mouse CD4+CD25− Tconv/Tresp cells were acquired as the dump population of CD4+ T cell remaining after CD25+ Treg cell purification.

All cells were cultured in RPMI 1640 with L-glutamine (Gibco, Thermo Fisher) and supplemented with 10% FBS (Gemini BioProducts), 10 nM HEPES (Life Technologies, Thermo Fisher) (San Diego, CA), 1% non-essential amino acids (Life Technologies, Thermo Fisher), and 1 mM sodium pyruvate (Life Technologies, Thermo Fisher).

### Antibodies

Flow cytometry and cell stimulation utilized the following antibodies from Thermo Fisher directed against mouse antigens: Foxp3 (FJK-16s), CD25 (PC61.5), CD45RB (16A), CD4 (GK1.5), CD8 (53-6.7), CD38 (2c11), CD28 (37.51), and CTLA-4 (UC10-4B9) and human antigens: CD28 (CD28.2), CD3 (OKT3), CD4 (RPA-T4), CD8 (SK1), CTLA-4 (14D3), CD28 (CD28.2), CD45RB (PD7/26), IL-10 (JES3-9D7), IL-37 (37D12), CD127 (eBioRDR5), AIM2 (14-6008-93) phospho-Smad3 S208 (PA5-1042450), phospho-caspase-1 S376 (PA5-38565), phosphor-NLRC4 S533 (MA5-31846), and phospho-PAK1/2/3 S144, S141, S139 (MAF-32130), and FOXP3 (3G3, SK3, and 236A/E7). FOXP3 (D608C) was purchased from Cell Signaling.

### Primeflow® RNA Assay

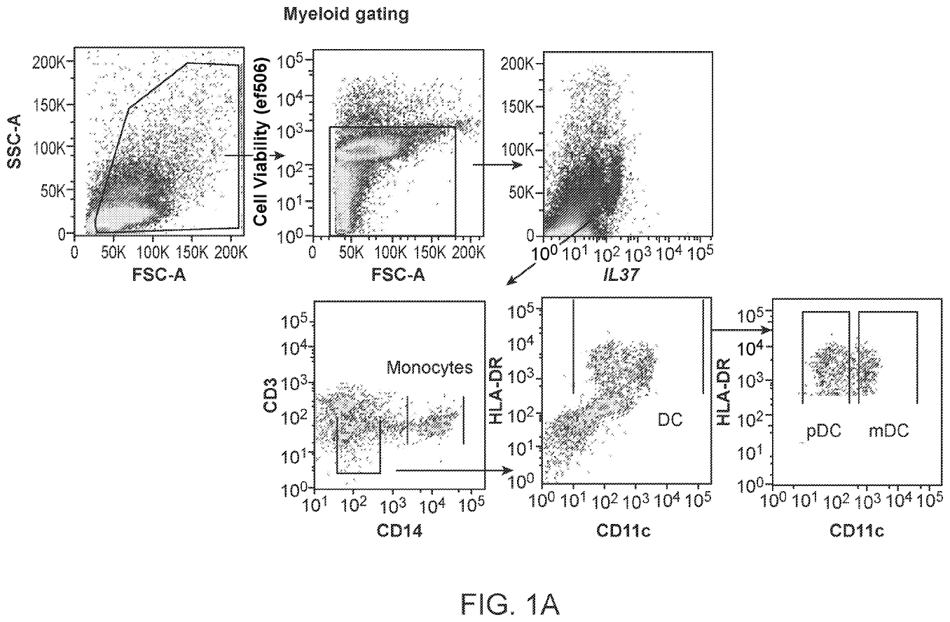

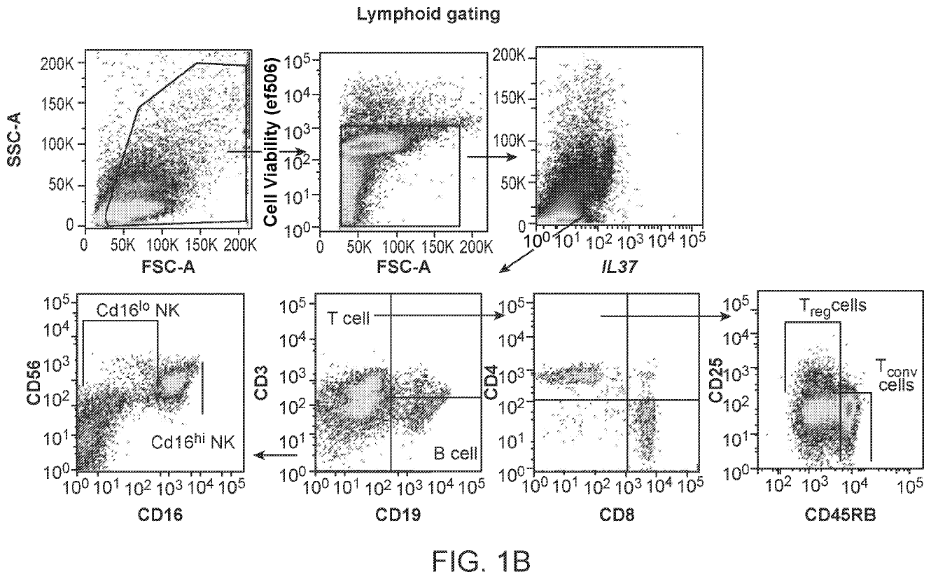

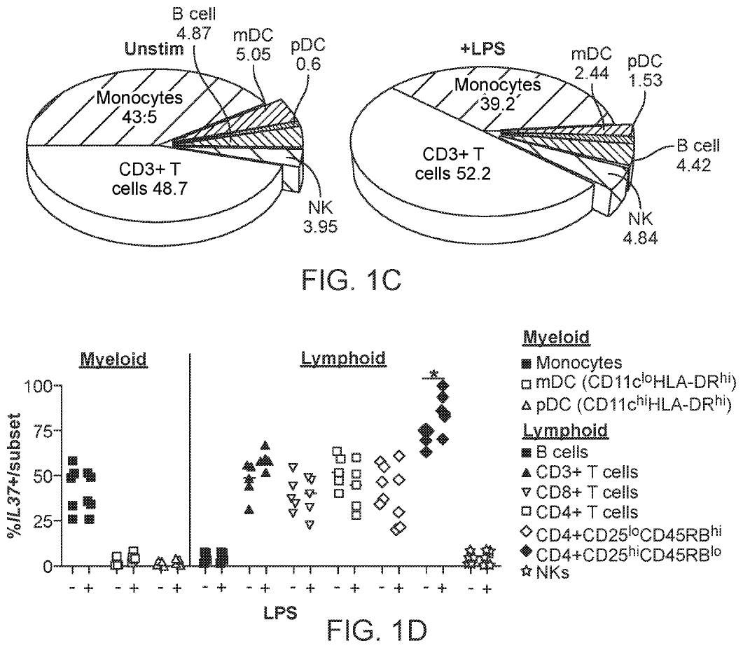

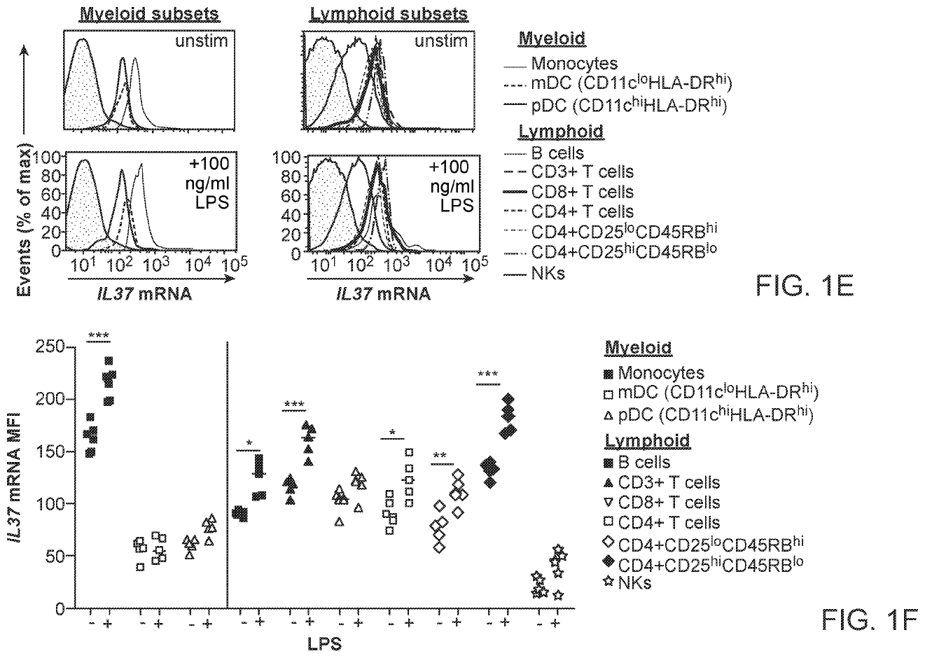

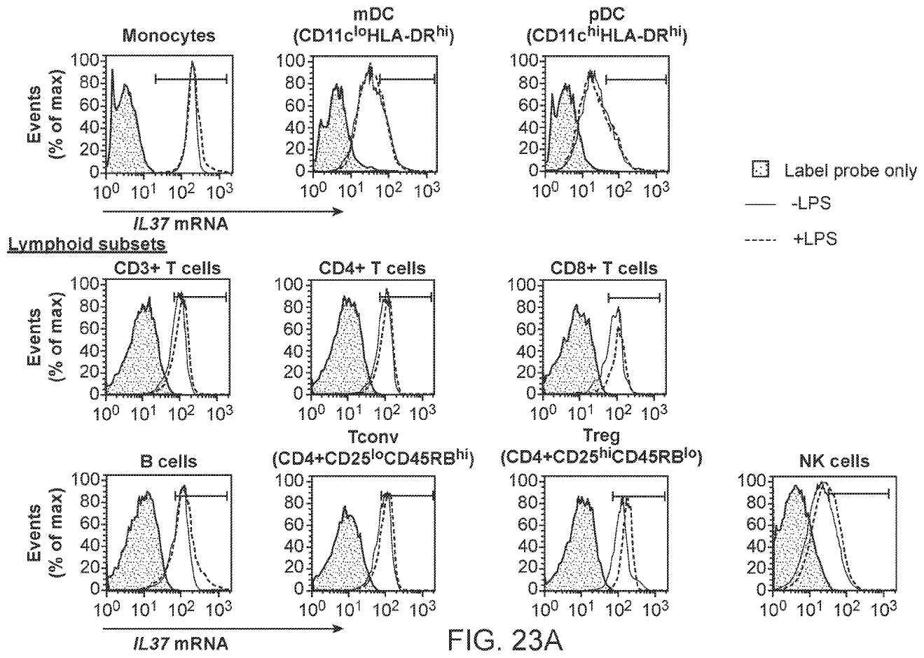

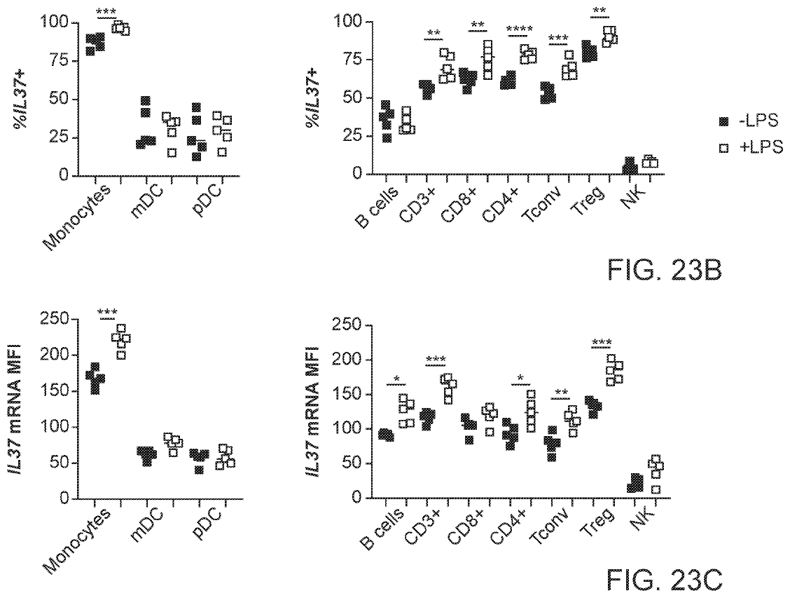

Human donor PBMCs were harvested and purified using Ficoll gradient centrifugation. Cells were then either left untreated or cultured with 100 ng/ml lipopolysaccharide (LPS) in complete RPMI 1640 media or resuspended in freeze media (RPMI+10% DMSO (Sigma)) and placed in a −80° C. freezer for later use. After 24 h fluorescent mRNA in situ hybridization was performed with the PrimeFlow® RNA Assay from Thermo Fisher with custom designed IL-37 mRNA specific probes according the manufacturer's instructions. The IL37 mRNA probe was designed by Thermo Fisher based on our published IL37 qRT-PCR primer sequences (Luo et. al., 2014) to obtain consistent results between the two different assays. For surface staining, the manufacturer's recommended amount (5 μl/test) of fluorescently conjugated antibody specific to the surface marker was added to each donor PMBC sample seeded at 1×10⁶ cells and resuspended in 100 μl of FACS buffer for 30 minutes at 4° C., then washed 2× with FACS buffer prior to being either analyzed or further processed. Immune cells were stained into two groups based on the manufacturer's provided immune antibody panels, one set for myeloid subsets and the other for lymphoid subsets (shown by the gating strategies in FIGS. 23A-B). Once gated on the individual subset, cells were analyzed for the expression of IL37 mRNA (AF437). The PrimeFlow®-specific antibodies for cell surface proteins, including CD3 (efluor450, UCHT1), CD4 (AF700, RPA-T4), CD8 (APC-efluor780, RPA-T8), CD11c (PE-efluor610, 3.9), CD14 (PECy7, 61D3), CD16 (PE, EBIOCB16), CD19 (PECy5.5), CD25 (PE-Cy7, BC96), CD45RB (AF488, PD7/26), CD56 (PE-Ef610, CMSSB), and HLA-DR (FITC, L243) were purchased from ThermoFisher. Fixable Viability Dye (FVD, eflour506) was used to eliminate dead cells. Flow cytometry of PrimeFlow® RNA samples was performed on a ZE5 Cell Analyzer (BioRad, Hercules, CA). Gating strategies of PrimeFlow® samples for specific cell analysis, such as the percentage IL-37+ cells are shown in FIGS. 1A-B. Further analysis of flow cytometry data was done using FlowJo_V10 software (Tree Star).

siRNA Knockdown in Primary Human T Cells

Purified primary human CD4+ T cells were used for siRNA knockdown of RUNX1, RPTOR, IL37, CREB1, CHK1, NLRP2, NLRP3, NLRP4, ASC, and NLRC4, and CD4+CD25+CD127^{dim} T cells for siRNA knockdown of FOXP3 and IL37. Cells were purified from healthy donor PBMCs as described above and then placed in culture in complete RPMI 1640 for at least 2 h prior to siRNA transfection via nucleofection using the Amaxa P3 Primary Cell 4D-Nucleofector X Kit L (Lonza) following the manufacturer's instructions. For the knockdown of RPTOR and IL-37, three different siRNAs (30 nM) against RPTOR and IL-37: A, B, and C (OriGene, Rockville, MD) were used separately or pooled, whereas for FOXP3 knockdown, only the pool of three different siRNAs (30 nM) against FOXP3: A, B, and C (OriGene) was used due to a limited number CD4+CD25+CD127^{dim} T cells. For the knockdown of NLRP3A, three to four siRNAs were pooled together, and for NLRC4 and NLRP4, Dharmacon SMARTpool was used. Single-sense and anti-sense siRNAs were used for NLRP2 and ASC (Ambion). Scrambled siRNA (OriGene, Ambion, and Dharmacon) was used as a control. Knockdowns were confirmed 24 hours after nucleofection by qRT-PCR or 48 hours later by western blot, and the relative density of the protein bands was determined using FIJI. As described above, the remaining T cells were then stimulated with anti-CD3/CD28 Dynabeads™ and assayed for FOXP3 expression or activated caspase-1 by flow cytometry. FOXP3 knockdown was confirmed by qRT-PCR 24 h following nucleofection, and the remaining cells were antibody stimulated using anti-CD3/CD28 Dynabeads™ and assayed for IL-37 and CTLA-4 expression using flow cytometry. All sequences for siRNA are located in Table 1 below.

TABLE 1
Exemplary siRNA of the Disclosure

| Gene | SIRNA | SEQ ID NO: |
|---|---|---|
| ASC | Sense-GCUCUUCAGUUUCACACCATT | SEQ ID NO: 1 |
| (Origene) | Antisense-UGGUGUGAAACUGAAGAGCTT | SEQ ID NO: 2 |
| Caspase-1 | Sense-GGUUCGAUUUUCAUUUGAG | SEQ ID NO: 3 |
| (Ambion) | Antisense-CUCAAAUGAAAAUCGAACC | SEQ ID NO: 4 |
| CHEK1 | A-AGAUGACACGAUUCUUUACCAAAT | SEQ ID NO: 5 |
| (OriGene) | B-AGAUUGUAGAUAUGAAGCGUGCCGT | SEQ ID NO: 6 |
| | C-GGAAUAGUACUUACGCAAUGCUCG | SEQ ID NO: 7 |
| CREB1 | Sense-CCGUAACUCUAGUACAGCU | SEQ ID NO: 8 |
| (Ambion) | Antisense-AGCUGUACUAGAGUUACGG | SEQ ID NO: 9 |
| Foxp3 | A-AGCGGACACUCAAUGAGAUCUACCA | SEQ ID NO: 10 |
| (OriGene) | B-UCAAGAUCAACCAAACCACCAUGGA | |
| | C-GCCAAACAGAGCCUUCACAACCAGC | SEQ ID NO: 11 |
| | | SEQ ID NO: 12 |
| IL-37 | A-ACACCAAACCUGCUCACUAAAAAAA | SEQ ID NO: 13 |
| (OriGene) | B-GAAUUUUCAUUUCAACCAGUUUGCA | SEQ ID NO: 14 |
| | C-AUAAACUCAACGUUGAAAAUGUCCT | SEQ ID NO: 15 |
| NLRC4 | SMARTpool | |
| (Dharmacon) | GACAUUACAUCCACUUAUA | SEQ ID NO: 16 |
| | UUAAAGGACUUGUACCAUA | SEQ ID NO: 17 |
| | GCACAUCACAUCUGUAACA | SEQ ID NO: 18 |
| | GUACACAGCAGGACGAAGA | SEQ ID NO: 19 |
| NLRP1 | A-GTCAAGAGAAGCTGGCCTGATTATGTGGA | SEQ ID NO: 20 |
| (Origene) | B-CCTGTGGCTACTGAGGTAGTTGACAAAGA | SEQ ID NO: 21 |
| | C-GAAGGAGTTCCAGCTTCTGCTCGCCAATA | SEQ ID NO: 22 |
| | D-CAGAACCTCATTCCTTCTTTGGAGCAGGC | SEQ ID NO: 23 |
| NLRP3 | A-GUACCUUUCGAGAAUCUCUAUUUGT | SEQ ID NO: 24 |
| (Origene) | B-CUGAAGCACCUGUUGUGCAAUCUGA | SEQ ID NO: 25 |
| | C-AGCAUCGGGUGUUGUUGUCAUCACA | SEQ ID NO: 26 |
| NLRP4 | SMARTpool | |
| (Dharmacon) | GCUCUGACGCAUACGGAUU | SEQ ID NO: 27 |
| | CUGAAAUGCUUCUGCGUAA | SEQ ID NO: 28 |
| | ACAACAAGAAGCUGACGUA | SEQ ID NO: 29 |
| | CCUAUCAAGCACACGCAAA | SEQ ID NO: 30 |
| RPTOR | A-GGAGAAGCGUCAGAUAGCGCGT | SEQ ID NO: 31 |
| (OriGene) | B-AGAACUACACGCAGUACAUCCCUCT | SEQ ID NO: 32 |
| | C-AGAUUGGACGACCAAAUAUUUCUGA | SEQ ID NO: 33 |
| RUNX1 | Sense-AGAACCAGGUUGCAAGAUU | SEQ ID NO: 34 |
| (Ambion) | Antisense-AAUCUUGCAACCUGGUUCU | SEQ ID NO: 35 |

### In Vitro T Cell Suppression Assay

For suppression assays containing Treg cells, CD4+CD25^{hi} T cells were purified from splenocytes of either WT or IL37 Tg mice using the Mouse CD4+CD25+ T cell Isolation Kit (Miltenyi). 100,000 purified CFSE-labeled WT CD4+CD25^{lo} T cell responders (Tresp) in 50 μl of complete RPMI 1640 were added to a round-bottom 96-well plate (CellStar, Dallas TX) containing heat-killed splenocytes (APC), 5 μg/ml soluble anti-CD3 antibody, and the indicated dilution of Treg cells all in a 150 μl of complete RPMI 1640 (Sarmento et. al., 2015). 3 d later, CFSE was used to measure proliferation/suppression of Tresp cells and analyzed by flow cytometry. Percent suppression was calculated using the equation, % suppression=100−(x/y) 100, X represents the division index (DI) of Tresp with Treg cells, and Y is the average DI of Tresp cells alone, as in (McMurchy and Levings, 2012).

### IL-37 ELISA

IL-37 secretion into the supernatant was analyzed by culturing 1×10⁶ cells in 1 ml of complete RPMI 1640 medium. Supernatants were then collected and analyzed using DuoSet® human IL-37/IL-1F7 ELISA kits (R&D Systems) to measure IL-37 protein abundance, according to the manufacturer's instructions. 1 and 2 ng/ml recombinant human IL-37 were used as controls to measure the sensitivity of the ELISA assay.

### Contact Hypersensitivity Assay

Six- to 8-week-old age- and sex-matched C57BL/6 WT mice were sensitized by application of 25 μl of 0.5% 2,4-dinitro-fluorobenzene (DNFB) (Sigma-Aldrich) in 4:1 (vol/vol) acetone/olive oil onto their shaved abdomen on day 0. As a control, the left ear was painted with 10 μl vehicle. Ear thickness was measured with an engineer's micrometer (Mitutoyo, Takatsu-ku, Japan). Results are expressed as net ear swelling, which was calculated by subtracting the thickness of the ear before treatment from the thickness of the ear after treatment.

### Contact Hypersensitivity Assay (Expanded Treg Cells)

Setup described above. On day 5, mice were injected i.v. with 1×PBS (vehicle), or 5×10⁵ MACS purified and expanded CD4+CD25^{hi} T cells from either donor WT or IL37 Tg 1 h prior to challenge with DNFB. Mice were challenged by applying 10 μl of 0.2% DNFB in 4:1 (vol/vol) acetone/olive oil onto their right ear. Clinical measurements and analyses described above

Tissue Collection from CHS for Purification and Analysis of Lymphocytes Populations

48 h after DNFB challenge ears, draining lymph nodes (dLN), and spleens were harvested from mice. Lymphocytes were obtained from vehicle and hapten-sensitized ears by enzymatic digestion with room-temperature HBSS media (Gibco) supplemented with 0.7 mg/ml collagenase D (Sigma-Aldrich) and density gradient separation was used following the previously described protocol (Benck et. al., 2016). dLN and spleen were macerated in a 40 μM filter (Falcon, Corning, NY). RBCs were lysed from splenocytes, and single cells from both dLNs and spleens were washed in 1×PBS and prepared for antibody staining (Osborne et. al., 2015). Single cell suspensions from ears, dLNs, and spleens were Fc blocked with purified anti-mouse CD16/CD32 monoclonal antibody (2.4G2) followed by staining for CD3, CD8, CD4, CD25, and CD45RB. Lymphocyte gating strategy is shown in FIG. 23B.

### Immunofluorescence (IF) and Imaging/Flow-Based Proximity Ligation Assay (PLA)

1×10⁵ Treg cells were cultured on poly-D-lysine coated Falcon® 8 chamber polystyrene vessel tissue culture treated glass slides (Corning, Corning, NY) for at least 30 min to allow firm adhesion. Cells were fixed by the addition of ice-cold fixative (4% paraformaldehyde and 0.5% glutaraldehyde in 1×PBS) and incubated for 30 min at room temperature in the dark, followed by permeabilization with 0.2% Triton X-100 in PBS for 15 min. Cells were then cultured with 10 μg/ml mouse anti-human IL-37 (IL-1F7) monoclonal antibody (7F1A1, Proteintech) and rabbit anti-human phospho-Smad3 (Ser423, Ser425) (ThermoFisher) in IF buffer (TBS plus goat serum cocktail) overnight at 4° C. in a humidifier chamber. Following 5-6 washes in 1×PBS, cells were incubated with an AF488-conjugated goat anti-mouse secondary antibody (1:500 dilution in imaging buffer) for 1 h at room temperature for IL-37 imaging alone. After 5-6 washes with PBS, ProLong™ Gold antifade reagent (Thermo Fisher) containing DAPI nuclear stain was added to each well.

For proximity ligation assay (PLA) experiments, after primary antibodies were washed out, cells were incubated for 1 h at 37° C. with an anti-rabbit PLUS probe (1:5 dilution; Duo92002; Sigma-Aldrich) anti-mouse MINUS probe (1:5 dilution; Duo92004; Sigma-Aldrich) and were washed two times with wash buffer A. Probes were then ligated for 30 min at 37° C. and washed two times in Buffer A and were amplified for 100 min at 37° C. in the dark with polymerase (Sigma-Aldrich). For flow-based PLA (Sigma-Aldrich), after amplification, cells were treated with 1× green detection buffer for 30 min at 37° C., then washed 2× with PLA wash buffer and prepared for flow analysis. After 2 washes with 1× Buffer B (Thermo Fisher), each well was added with ProLong™ Gold antifade reagent (Thermo Fisher) containing DAPI nuclear stain. Images were obtained with an Olympus FV1000 FCS/RICS system (Olympus, Tokyo, Japan). For analysis, 7 different 100×/1.4 Oil Plan-Apochromatic objective fields of view were imaged per well per experiment. Each field was measured for IL-37 fluorescence intensity using the FIJI measurement application. To analyze the cytoplasmic vs. nuclear localization of IL-37 within the T cells, DAPI and IL-37 overlapped images was used. Along with single-slice full field images, 0.25 μM slice z-stack images were taken to confirm cytoplasmic vs. nuclear localization of IL-37. Colocalization of IL-37 and DAPI was assessed by Pearson's correlation coefficient using FIJI. Line intensity profiles were created using a Plot profile in FIJI to measure differences in fluorescence across a cell by drawing a line spanning the diameter of the cell, and then the staining intensity measurements across the line were then entered into Prism 8 (GraphPad, Lo Jolla, CA). 3D fluorescent maps were created using a Surface plot in FIJI.

### Phosphoprotein Expression

Cell lysates from purified WT and IL37 Tg CD4+CD25+CD127^{dim} Treg cells were assayed using a Phospho Explorer antibody microarray (Full Moon, Sunnyvale, CA) that contains 1,318 antibodies. A single slide contains two replicates of each antibody printed on a coated glass microscope slide and multiple positive and negative controls. The slides were prepared according to the manufacturer's instructions, and Fullmoon scanned the slides. Fluorescence intensity for each antibody was measured using FIJI, and background intensity was subtracted from each fluorescent measurement before calculating fold change.

### Inflammasome RT Profiler PCR Array

The Inflammasomes RT² Profiler PCR Array (Qiagen, Valencia, CA) was used to analyze gene expression of 84 inflammasome components and signaling pathways in unstimulated purified human CD4+CD25+CD127^{dim} Treg cells and CD4+CD25−CD127^{hi} Tconv cells according to the manufacturer's instructions. Experiments were performed a minimum of three times for Treg and Tconv cells. PCR array data were analyzed using the GeneGlobe Data Analysis Center on QIAGEN's website at http://www.qiagen.com/kr/shop/genes-and-pathways/data-analysis-center-overview-page/.

### Ingenuity® Pathway Analysis (IPA®)

Pathway analysis was done using Ingenuity Pathway Analysis (IPA) to determine the association of Hsp90 with NLR-family members and inflammasome-associated molecules: http://www.ingenuity.com/. IPA® results in Table 3.

### Jurkat E6 Transduction

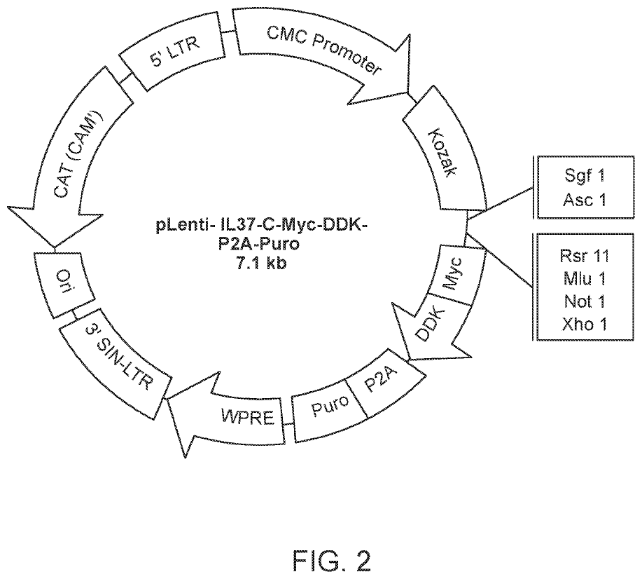

Jurkat cells were transduced with lentivirus made from transfecting 293T cells transfected with either an empty vector or pLenti-IL37-C-Myc-DDK-P2A-Puro vector (IL37 OE) (Origene) together with the Lenti-V-pak packaging kit (Origene). A schematice of the pLenti-IL37-C-Myc-DDK-P2A-Puro vector is depicted in FIG. 2. Supernatants containing lentiviral particles were harvested 48 hours after transfection. The presence of lentivirus in the supernatant was confirmed using Lenti-X GoStix Plus (Takara). 1×10⁶ Jurkat cells were transduced using 1 ml lentivirus-containing supernatants and 8 μg/ml polybrene and incubated for 24 hours at 37° C. Media was then changed to RPMI1640 supplemented with pen-strep and 10% FCS. Drug selection was started 48 hours after transduction with 0.5 μg/ml puromycin. Jurkat cells were bulk selected, and overexpression was confirmed by western blot.

### Flow Cytometry: Surface and Intracellular Antigen Staining, Activation, and Proliferation

Intracellular FOXP3 and cytokines were assayed by flow cytometry. Prior to fix/permeabilization, cells were surface-stained at 4° C. for 30 minutes. Purified lymphocytes were washed with FACS buffer (5% BSA and 1×PBS) and then prepared for surface or intracellular staining. For surface staining, 1 μg or the manufacturer's recommended amount of fluorescently conjugated antibody specific to the surface marker to be visualized was added to 1×10⁶ cells resuspended in 100 μl of FACS buffer for 30 minutes at 4° C., then washed 2× with FACS buffer prior to being either analyzed or further processed. In the latter, following 2× wash with FACS buffer, cells were immediately fixed in 4% paraformaldehyde (PFA) and permeabilized with 90% methanol, followed by staining. For IL-10 intracellular cytokine staining, the FOXP3 Cytoperm/Cytofix staining kit (BD Pharmingen, San Diego, CA) was used after cells were stimulated for 24 hours on 1 μg/ml anti-CD3-coated plates with soluble 1 μg/ml anti-CD28 antibody or anti-CD3/CD28 conjugated Dynabeads (Gibco, Thermo Fisher) and treated in culture for 6 hours with BD GolgiStop™ (Franklin Lakes, NJ). T cell proliferation was measured by staining cells with 5 μM CFSE (Molecular Probes, Eugene, OR) prior to cell culture and analyzing them using flow cytometry

### RNA Extraction and Quantitative RT-PCR Analysis

Cells were stimulated for 24 hours on anti-CD3/CD28-coated plates, as described above. Total RNA was extracted from Jurkat cells using the RNeasy Plus Mini Kit (Qiagen) and subsequently reverse-transcribed using the iScript cDNA Synthesis Kit (Bio-Rad). Quantitative RT-PCR (qRT-PCR) was performed with Power Up SYBR Green PCR Master Mix (Applied Biosystems, Foster City, CA) on the AriaMx Real-Time PCR system (Agilent Technologies, Santa Clara, CA). Primer sets used for human cells were the following:

GAPDH
forward-
(SEQ ID NO: 178)
5′-TGC ACC ACC AAC TGC TTA GC-3′;
reverse-
(SEQ ID NO: 179)
5′-GGC ATG GAC TGT GGT CAT GAG-3′;
IL-37
forward-
(SEQ ID NO: 180)
5′-GCA TTC ATG ACC AGG ATC AC-3′;
reverse-
(SEQ ID NO: 181)
5′-CAA AGA AGA TCT CTG GGC GTA-3′;
FOXP3
forward-
(SEQ ID NO: 182)
5′-CAG AGC TCC TAC CCA CTG CT-3′;
reverse-
(SEQ ID NO: 183)
5′-CTT CTC CTT CTC CAG CAC CA-3′;
GITR
forward-
(SEQ ID NO: 184)
5′-CAT GTG TGT CCA GCC TGA AT-3′;
reverse-
(SEQ ID NO: 185)
5′-GGC ACA GTC GAT ACA CTG GA-3′;
CTLA-4
forward-
(SEQ ID NO: 186)
5′-CTC TAC ATC TGC AAG GTG GAG C-3′;
reverse-
(SEQ ID NO: 187)
5′-AGA GGA GGA AGT CAG AAT CTG GG-3′;
TGF-β
forward-
(SEQ ID NO: 188)
5′-CAC CTG GAG CTG TAC CAG AA-3′;
reverse-
(SEQ ID NO: 189)
5′-TGC AGT GTG TTA TCC CTG CT-3′;
IL17
forward-
(SEQ ID NO: 190)
5′-ATA TTG GGG CTT GCC TTT CT-3′;
reverse-
(SEQ ID NO: 191)
5′-GTG TAA TTC CAG GGG GAG GT-3′,
STAT3
forward-
(SEQ ID NO: 192)
5′-AGA GAA ATG AGT GAG TGT GGG-3′;
reverse-
(SEQ ID NO: 193)
5′-ACA GGA GGT GTT CCC CTT TGC-3′,
and
GATA3
forward-
(SEQ ID NO: 194)
5′-AGG CAG GGA GTG TGT GAA CT-3′;
reverse-
(SEQ ID NO: 195)
5′-GTG GTT GTG GTG GTC TGA CA-3′

Primer sets used for mouse cells were the following:

GAPDH forward-
(SEQ ID NO: 196)
5′C ACC ACC AAC TGC TTA GC-3′;
reverse-
(SEQ ID NO: 197)
5′-GGC ATG GAC TGT GGT CAT GAG-3′;
IL37 forward-
(SEQ ID NO: 198)
5′-GCA TTC ATG ACC AGG ATC AC-3′;
reverse-
(SEQ ID NO: 199)
5′-CAA AGA AGA TCT CTG GGC GTA-3′;
FOXP3 forward-
(SEQ ID NO: 200)
5′-CCA GGA CAG ACC ACA TTC A-3′;
reverse-
(SEQ ID NO: 201)
5′-CTG GAC ACC CAT TCC AGA CT-3′.

Additional primer sets are listed in Tables 2.1 and 2.2 below. Quantification was measured using a change-in-cycling threshold (ΔCt) and is shown relative to GAPDH mRNA expression.

TABLE 2.1
Exemplary Primers of the Disclosure

| Human | Primer sets 1 | SEQ ID |
|---|---|---|
| Gene | (5′-3′) | NO: |
| ATG7 | F1-AGCGGCGGCAAGAAATAATG | SEQ ID |
| | | NO: 36 |
| | R1-AACCCAACATCCAAGGCACT | SEQ ID |
| | | NO: 37 |
| BRG1 | F1-GGGTAGCAGCAGATGTAGTTT | SEQ ID |
| | | NO: 38 |
| | R1-CCAGTCACAAACAGTCCTACA | SEQ ID |
| | | NO: 39 |
| Caspase-1 | F1-CCTGGTGTGGTGTGGTTTA | SEQ ID |
| | | NO: 40 |
| | R1-ATCCTTCTCTATGTGGGCTTTC | SEQ ID |
| | | NO: 41 |
| c-Fos | F1-GGTGCATTACAGAGAGGAGAAA | SEQ ID |
| | | NO: 42 |
| | R1-GTGTGTTTCACGCACAGATAAG | SEQ ID |
| | | NO: 43 |
| CHEK1 | F1-TCTATGGTCACAGGAGAGAAGG | SEQ ID |
| | | NO: 44 |
| | R1-CATGCCTATGTCTGGCTCTATTC | SEQ ID |
| | | NO: 45 |
| C-Jun | F1-CCTGATGTACCTGATGCTATGG | SEQ ID |
| | | NO: 46 |
| | R1-CCTCCTGAAACATCGCACTAT | SEQ ID |
| | | NO: 47 |
| CREB1 | F1-GAACCAGCAGAGTGGAGATG | SEQ ID |
| | | NO: 48 |
| | R1-GGCATAGATACCTGGGCTAATG | SEQ ID |
| | | NO: 49 |
| CTLA4 | F1-GACAATGGCTTACTCCAGGAGAC | SEQ ID |
| | | NO: 50 |
| | R1-TGCTGCCTTCTTCTGTCCATG | SEQ ID |
| | | NO: 51 |
| FOXP3 | F1-CAGAGCTCCTACCCACTGCT | SEQ ID |
| | | NO: 52 |
| | R1-CTTCTCCTTCTCCAGCACCA | SEQ ID |
| | | NO: 53 |
| Foxp3 | F1-CCAGGACAGACCACATTCA | SEQ ID |
| | | NO: 54 |
| | R1-CTGGACACCCATTCCAGACT | SEQ ID |
| | | NO: 55 |
| GAPDH | F1-TGCACCACCAACTGCTTAGC | SEQ ID |
| | | NO: 56 |
| | R1-GGCATGGACTGTGGTCATGAG | SEQ ID |
| | | NO: 57 |
| Gapdh | F1-CACCACCAACTGCTTAGC | SEQ ID |
| | | NO: 58 |
| | R1-GGCATGGACTGTGGTCATGAG | SEQ ID |
| | | NO: 59 |
| GATA3 | F1-AGGCAGGGAGTGTGTGAACT | SEQ ID |
| | | NO: 60 |
| | R1-GTGGTTGTGGTGGTCTGACA | SEQ ID |
| | | NO: 61 |
| Gata3 | F1-CAGCTGCCAGATAGCATGAA | SEQ ID |
| | | NO: 62 |
| | R1-CTTGGGCCTCGACTTACATC | SEQ ID |
| | | NO: 63 |
| GITR | F1-CATGTGTGTCCAGCCTGAAT | SEQ ID |
| | | NO: 64 |
| | R1-GGCACAGTCGATACACTGGA | SEQ ID |
| | | NO: 65 |
| GSK3B | F1-GTAACCCAGGGAGGTCAATAAA | SEQ ID |
| | | NO: 66 |
| | R1-GCCTCTACATCACTAGCACTAAC | SEQ ID |
| | | NO: 67 |
| GTF2IRD1 | F1-GAGAAAGCGGGTCTCGGAAG | SEQ ID |
| | | NO: 68 |
| | R1-ATTGGCCATTGCACGAGTGA | SEQ ID |
| | | NO: 69 |
| GSK3B | F1-GTAACCCAGGGAGGTCAATAAA | SEQ ID |
| | | NO: 70 |
| | R1-GCCTCTACATCACTAGCACTAAC | SEQ ID |
| | | NO: 71 |
| HDAC3 | F1-TGATCGATTGGGCTGCTTTA | SEQ ID |
| | | NO: 72 |
| | R1-CAGCACGAGTAGAGGGATATTG | SEQ ID |
| | | NO: 73 |
| Helios | F1-TGAAGAGTGTGACAGGAAACC | SEQ ID |
| | | NO: 74 |
| | R1-CACCTCGCTGCTCTCAATTA | SEQ ID |
| | | NO: 75 |
| Ikaros | F1-GAGGAAGGTGTGTGGAGATTC | SEQ ID |
| | | NO: 76 |
| | R1-GCCTAGGTGGTTGAGAGTAATG | SEQ ID |
| | | NO: 77 |
| IL10 | F1-AGAACAGCTGCACCCACTTC | SEQ ID |
| | | NO: 78 |
| | R1-GCATCACCTCCTCCAGGTAA | SEQ ID |
| | | NO: 79 |
| IL17 | F1-ATATTGGGGCTTGCCTTTCT | SEQ ID |
| | | NO: 80 |
| | R1-GTGTAATTCCAGGGGGAGGT | SEQ ID |
| | | NO: 81 |
| IL-37 | F1-GCATTCATGACCAGGATCAC | SEQ ID |
| | | NO: 82 |
| | R1-CAAAGAAGATCTCTGGGCGTA | SEQ ID |
| | | NO: 83 |
| IRF4 | F1-CTTTGAGGAATTGGTCGAGAGG | SEQ ID |
| | | NO: 84 |
| | R1-GAGAGCCATAAGGTGCTGTCA | SEQ ID |
| | | NO: 85 |
| Irf4 | F1-TCCGACAGTGGTTGATCGAC | SEQ ID |
| | | NO: 86 |
| | R1-CCTCACGATTGTAGTCCTGCTT | SEQ ID |
| | | NO: 87 |
| NK-kB | F1-CACCCTGACCTTGCCTATTT | SEQ ID |
| | | NO: 88 |
| | R1-AGCTGCTTGGCGGATTAG | SEQ ID |
| | | NO: 89 |
| NLRC4 | F1-TGGGATCACCTTTGACCTTTC | SEQ ID |
| | | NO: 90 |
| | R1-GGCTCGGCTATTGTCCTTTAT | SEQ ID |
| | | NO: 91 |
| NLRP4 | F1-CTGAAGGACGAAGGACTGAAA | SEQ ID |
| | | NO: 92 |
| | R1-AGCCAGCAGCAGTGATAAA | SEQ ID |
| | | NO: 93 |
| PDC-E2 | F1-GTTCCCATCGGAGCGATCAT | SEQ ID |
| | | NO: 94 |
| | R1-GTGCTGCTGAGGAATCCAGT | SEQ ID |
| | | NO: 95 |
| PDC-E2 | F1-GTTCCCATCGGAGCGATCAT | SEQ ID |
| | | NO: 96 |
| | R1-GTGCTGCTGAGGAATCCAGT | SEQ ID |
| | | NO: 97 |
| RORyT | F1-GGAAGTGGTGCTGGTTAGGA | SEQ ID |
| | | NO: 98 |
| | R1-TGATGAGAACAAGGGCTGTG | SEQ ID |
| | | NO: 99 |
| RoryT | F1-GAAAGCAGGAGCAATGGAAG | SEQ ID |
| | | NO: 100 |
| | R1-AGAGGGCAATCTCATCCTCA | SEQ ID |
| | | NO: 101 |
| RPTOR | F1-AAGGACAACGGCCACAAGTA | SEQ ID |
| | | NO: 102 |
| | R1-ACGATCACGGCGAGAATGAA | SEQ ID |
| | | NO: 103 |
| RUNX1 | F1-CTGCCCATGGCTTTCAAGGT | SEQ ID |
| | | NO: 104 |
| | R1-GCCGAGTAGTTTTCATCATTGCC | SEQ ID |
| | | NO: 105 |
| SATB1 | F1-CCTTCCCAAGTACACCATCATC | SEQ ID |
| | | NO: 106 |
| | R2-CTGCCACATCGACCTCTAAAC | SEQ ID |
| | | NO: 107 |
| SCAI | F1-CAGGTCGTTCTGATAGTGAAGG | SEQ ID |
| | | NO: 108 |
| | R1-CTTGTGGGACTGATTTCGTTTG | SEQ ID |
| | | NO: 109 |
| SREBP-2 | F1-GGAGACCATGGAGACCCTCA | SEQ ID |
| | | NO: 110 |
| | R1-GTCAGGGAACTCTCCCACTTG | SEQ ID |
| | | NO: 111 |
| STAT3 | F1-AGAGAAATGAGTGAGTGTGGG | SEQ ID |
| | | NO: 112 |
| | R1-ACAGGAGGTGTTCCCCTTTGC | SEQ ID |
| | | NO: 113 |
| TBET | F1-CCAGGGAACCGCTTATATGT | SEQ ID |
| | | NO: 114 |
| | R1-CTGGGTCACATTGTTGGAAG | SEQ ID |
| | | NO: 115 |
| TGFB | F1-CACCTGGAGCTGTACCAGAA | SEQ ID |
| | | NO: 116 |
| | R1-TGCAGTGTGTTATCCCTGCT | SEQ ID |
| | | NO: 117 |
| WDR48 | F1-CCATCACCGGCAGAACACAG | SEQ ID |
| | | NO: 118 |
| | R1-ATCCAGCTGCAGAGCATTGA | SEQ ID |
| | | NO: 119 |

TABLE 2.2
Exemplary Primers of the Disclosure

| Human | Primer sets 2 | SEQ ID |
|---|---|---|
| Gene | (5′-3′) | NO: |
| ATG7 | F2-GCGGCGGCAAGAAATAATGG | SEQ ID |
| | | NO: 120 |
| | R2-AACCCAACATCCAAGGCACT | SEQ ID |
| | | NO: 121 |
| BRG1 | F2-CACAAAGTGCTGCTGTTCTG | SEQ ID |
| | | NO: 122 |
| | R2-TTCCATCAAGCCTGAGGTATTT | SEQ ID |
| | | NO: 123 |
| Caspase-1 | F2-GGATAAGACCCGAGCTTTGATT | SEQ ID |
| | | NO: 124 |
| | R2-CTGCCAGGTAACTGTCTTCTTC | SEQ ID |
| | | NO: 125 |
| c-Fos | F2-GGTGCATTACAGAGAGGAGAAA | SEQ ID |
| | | NO: 126 |
| | R2-GTGTGTTTCACGCACAGATAAG | SEQ ID |
| | | NO: 127 |
| c-Fos | F2-GGACTCAAGTCCTTACCTCTTC | SEQ ID |
| | | NO: 128 |
| | R2-CCTGGCTCAACATGCTACTA | SEQ ID |
| | | NO: 129 |
| CHEK1 | F2-GGTTGACTTCCGGCTTTCTAA | SEQ ID |
| | | NO: 130 |
| | R2-TCTTCTGGCTGCTCACAATATC | SEQ ID |
| | | NO: 131 |
| CHEK1 | F2-TCTATGGTCACAGGAGAGAAGG | SEQ ID |
| | | NO: 132 |
| | R2-CATGCCTATGTCTGGCTCTATTC | SEQ ID |
| | | NO: 133 |
| C-Jun | F2-CCTGATGTACCTGATGCTATGG | SEQ ID |
| | | NO: 134 |
| | R2-CCTCCTGAAACATCGCACTAT | SEQ ID |
| | | NO: 135 |
| c-Jun | F2-CACAGAGAGACAGACTTGAGAAC | SEQ ID |
| | | NO: 136 |
| | R2--ACTTGGATACCCTTGGCTTTA | SEQ ID |
| | | NO: 137 |
| CREB1 | F2-GGCCATCTGCGTATCTTCTT | SEQ ID |
| | | NO: 138 |
| | R2-AAGGGCCTAGTACCCAGAATA | SEQ ID |
| | | NO: 139 |
| CREB1 | F2-GAACCAGCAGAGTGGAGATG | SEQ ID |
| | | NO: 140 |
| | R2-GGCATAGATACCTGGGCTAATG | SEQ ID |
| | | NO: 141 |
| GTF2IRD1 | F2-CAAGAGAAAGCGGGTCTCGG | SEQ ID |
| | | NO: 142 |
| | R2-ACATTGGCCATTGCACGAGT | SEQ ID |
| | | NO: 143 |
| GSK3B | F2-CCTCTGGCTACCATCCTTATTC | SEQ ID |
| | | NO: 144 |
| | R2-CGGTCTCCAGTATTAGCATCTG | SEQ ID |
| | | NO: 145 |
| GSK3B | F2-GTAACCCAGGGAGGTCAATAAA | SEQ ID |
| | | NO: 146 |
| | R2-GCCTCTACATCACTAGCACTAAC | SEQ ID |
| | | NO: 147 |
| HDAC3 | F2-GCTGGTAGAAGAGGCCATTAG | SEQ ID |
| | | NO: 148 |
| | R2-GGTGCTGACATCTGGATGAA | SEQ ID |
| | | NO: 149 |
| Helios | F2-GCAGGGAAACTGCTGATAGT | SEQ ID |
| | | NO: 150 |
| | R2-GTGGAATCCAGGCAGCTATT | SEQ ID |
| | | NO: 151 |
| Ikaros | F2-GACAGAGGATCAAGGGCTTTA | SEQ ID |
| | | NO: 152 |
| | R2-AGAAGGCTCTCACCTTAGAGATA | SEQ ID |
| | | NO: 153 |
| Ikaros | F2-GAGGAAGGTGTGTGGAGATTC | SEQ ID |
| | | NO: 154 |
| | R2-GCCTAGGTGGTTGAGAGTAATG | SEQ ID |
| | | NO: 155 |
| IL-37 | F2-GCATTCATGACCAGGATCAC | SEQ ID |
| | | NO: 156 |
| | R2-CAAAGAAGATCTCTGGGCGTA | SEQ ID |
| | | NO: 157 |
| NF-KB | F2-TGGGACCAGCAAAGGTTATT | SEQ ID |
| | | NO: 158 |
| | R2-GATCCCATCCTCACAGTGTTT | SEQ ID |
| | | NO: 159 |
| NLRC4 | F2-GGACGAAGACTCAGCAGTTTAT | SEQ ID |
| | | NO: 160 |
| | R2-CAGGCTGCTATAAGTGGATGTAA | SEQ ID |
| | | NO: 161 |
| NLRP4 | F2-GTTCCTGGTCACTGTCTCTTT | SEQ ID |
| | | NO: 162 |
| | R2-GCTCCTCCAGATACCACATAAG | SEQ ID |
| | | NO: 163 |
| PDC-E2 | F2-ACTGGATTCCTCAGCAGCAC | SEQ ID |
| | | NO: 164 |
| | R2-GCCTGAGCAGAAGGTGTAGG | SEQ ID |
| | | NO: 165 |
| RPTOR | F2-GACCTCGTGAAGGACAACGG | SEQ ID |
| | | NO: 166 |
| | R2-TTGACGATCACGGCGAGAAT | SEQ ID |
| | | NO: 167 |
| SATB1 | F2-GAATCTCCCAGGCGGTATTT | SEQ ID |
| | | NO: 168 |
| | R2-CCTCTTCCTTTCGGAGGATTT | SEQ ID |
| | | NO: 169 |
| SCAI | F2-GCATACCACCTGCCCTTTAT | SEQ ID |
| | | NO: 170 |
| | R2-CCTTTGCTTTGCTCTGTTCTTAG | SEQ ID |
| | | NO: 171 |
| SCAI | F2-CAGGTCGTTCTGATAGTGAAGG | SEQ ID |
| | | NO: 172 |
| | R2-CTTGTGGGACTGATTTCGTTTG | SEQ ID |
| | | NO: 173 |
| SREBP-2 | F2-GGGAGACATCGACGAGATGC | SEQ ID |
| | | NO: 174 |
| | R2-CAGGAAAGGAGCTACACAGC | SEQ ID |
| | | NO: 175 |
| WDR48 | F2-CCATCACCGGCAGAACACA | SEQ ID |
| | | NO: 176 |
| | R2-AGSTGCAGAGCATTGACTCC | SEQ ID |
| | | NO: 177 |

### Surface and Intracellular Flow Cytometry and FAM-FLICA® Caspase-1 Staining

Purified lymphocytes from either humans or mice were washed with FACS buffer (5% BSA (Sigma-Aldrich) and 1×PBS), and then prepared for surface or intracellular staining, as described (Osborne and Wetzel, 2012). For surface staining, 1 μg or the manufacturer-recommended amounts of fluorescently conjugated antibody specific to the surface marker to be visualized was added to 1×10⁶ cells resuspended in 100 μl of FACS buffer for 30 min at 4° C., then washed 2× with FACS buffer prior to being either analyzed or further processed. For intracellular cytokine staining (ICS), the FOXP3 Cytoperm/Cytofix staining kit (BD Pharmingen, San Diego, CA) was used after cells were stimulated for 24 h on 1 μg/ml anti-CD3-coated plates with soluble 1 μg/ml anti-CD28 antibody or anti-CD3/CD28 conjugated Dynabeads™ (Gibco, Thermo Fisher). For measuring proliferation, cells were stained with 5-μM CFSE (Molecular Probes, Eugene, OR) prior to cell culture and analyzed using flow cytometry. Flow-based measurement of caspase-1 activation was done using the FAM-FLICA® Caspase-1 Assay kit #98 following the manufacturer's instructions (ImmunoChemistry Tech, Bloomington, MN). For flow cytometry experiments using the caspase-1 inhibitor Ac-YVAD-CMK, cells were cultured with 50 μM Ac-YVAD-CMK for 2 h prior to stimulation, 24 h (mouse), and 48 h (human), with anti-CD3/CD28 conjugated Dynabeads™ and then prepared for flow cytometry analyses. For experiments using the PAK1/2/3 inhibitor FRAX597, cells were cultured with 1 μM FRAX597 for either 24 h (flow and qRT-PCR) or 48 h (immunoblotting) and then prepared for analysis. For flow cytometry or qRT-PCR experiments using IL-6 treatment, human and mouse cells were cultured with 20 ng/ml IL-6 (Peprotech) and anti-CD3/CD28 conjugated Dynabeads™ and then prepared for analyses. For flow cytometry and qRT-PCR experiments using the flagellin, cells were cultured with 100 ng/ml flagellin for 24 h and then prepared for analyses. During and after the preparation of cells for flow cytometry analyses, cells were placed in FACS buffer. All acquisitions were performed on a Galios® or Galios® 561 cytometer equipped with Kaluza software (Beckman-Coulter, Indianapolis, IN), and all data were analyzed with FlowJo_V10 software (Tree Star, Ashland, OR).

### Western Blot, Immunoprecipitation (IP) and Immunoblot Analysis

For immunoblot analysis, cells were lysed in RIPA buffer (50 mM Tris-HCL pH 7.4, 150 mM NaCl, 1 mM PMSF, 1 mM EDTA, 5 μg/ml Aprotinin, 5 g/ml Leupeptin, 1% Triton x-100, 1% Sodium deoxycholate, 0.1% SDS) (Sigma-Aldrich). For IP of Smad3, cells were lysed in Pierce® IP Lysis Buffer (Thermo) and prepared using the Thermo Immunoprecipitation containing Dynabeads™ Protein G kit according to the manufacturer's instructions. The Smad3 IP was performed using the rabbit monoclonal antibody against Smad3 (C67H9) (Thermo). Hsp90 IP using mouse monoclonal antibody against Hsp90 (610418) (BD Biosciences). 20 μg of sample was run on a 4-15% Mini-Protean TGX precast gel (Bio-Rad) and then transferred overnight at 4° C. and 30 V to a PVDF membrane using a Bio-Rad Trans-Blot® transfer cell. Following 1 h of blocking with blocking buffer (5% milk/Tween 20 in TBS), membranes were incubated with primary antibody overnight at 4° C. For siRNA knockdown blots, mouse β-actin monoclonal antibody (Sigma-Aldrich) at a dilution of 1:10,000 was used for the loading control. For Smad3 IP, rabbit anti-human IL-37 (IL-1F7) polyclonal antibody (Invitrogen, Thermo) at a dilution of 1:1000 was used to detect IL37. HRP-conjugated anti-rabbit (1:5000) and anti-mouse IgG (1:5000) were purchased from Sigma-Aldrich. The SuperSignal West Pico Kit (Thermo Fisher) was used according to the manufacturer's instructions for the chemiluminescent detection of the proteins. Protein expression was further analyzed by measuring the relative densities using FIJI (National Institutes of Health: http://rsb.info.nih.gov/ij/) or LI-COR Image Studio™ Software.

### In Vitro T Cell Suppression Assay

Human CD4+CD127loCD25+ T cell Isolation Kit (Miltenyi) was used to isolate human CD4+CD25^{lo} T cell responders (Tresp) and human CD4+CD25^{hi} T cells (human primary Treg cells) for suppression assays (Osborne et. al., 2022) for suppression assays. For mouse cells, the mouse CD4+CD25+ T cell Isolation Kit (Miltenyi) was used to isolate mouse CD4+CD25^{lo} T cell responders (Tresp) and mouse CD4+CD25^{hi} Treg cells from WT or IL37 Tg mouse splenocytes. 100,000 purified CFSE-labeled WT CD4+CD25^{lo} T cell responders (Tresp) in 50 μl of complete RPMI 1640 were added to a round-bottom 96-well plate (CellStar, Dallas TX) containing heat-killed splenocytes (APC) (as the source for antigen-presenting cells), 5 μg/ml soluble anti-CD3 antibody, and the indicated dilution of Treg cells all in a 150 μl of complete RPMI 1640. 5 days later, CFSE was used to measure proliferation/suppression of Tresp cells and analyzed by flow cytometry. Percent suppression was calculated using the equation, % suppression=100−(x/y)100, X represents the division index (DI) of Tresp with Treg cells, and Y is the average DI of Tresp cells alone, as in (McMurchy and Levings, 2012).

### Treg Cell Expansion

For mouse Treg cell expansion, spleens were harvested from either a 6- to 8-week-old age- and sex-matched C57BL/6 WT or IL37 Tg mouse. Spleens in 10 ml of 1×PBS were macerated using a 10 ml plastic syringe (Fisher Scientific) on 40 μm filters in a six-well plate. Splenocytes were then put through the same 40 μm filter into a 50 ml conical, and the six-well plate was washed with 10 ml 1×PBS and placed in the splenocyte solution in the 50 ml conical. Spin down at 1500 rpm for 10 min. Following the spin solution was aspirated, leaving only the splenocyte pellet. The splenocyte pellet was resuspended in 5 ml of RBC lysis buffer for 3 minutes then the buffer was neutralized by the addition of 15 ml of complete media (1×PBS+10% FBS). Spin down at 1500 rpm for 5 min. Following the spin solution was aspirated, leaving only the splenocyte pellet. Resuspend in 10 ml of 1×PBS+10% FBS and count using a hemacytometer. Following counting, splenocytes were prepared for Treg purification using a Miltenyi mouse CD4+CD25+ T cells purification kit. Purification followed the Miltenyi mouse CD4+CD25+ T cells purification kit protocol. After purification, 5×10⁵ cells/ml Treg cells in complete RPMI media supplemented with 1000 U/ml mouse IL-2 were then plated on 24-well plates coated with 1 μg/ml anti-CD3 (145-2C11) and 5 μg/ml anti-CD28 (37.51). Over the course of 10-day cultures, each well was split once cell numbers reached 1×10⁶ cells/ml, and 100 U/ml IL-2 supplement complete media was added. At day 10, cells were counted, and Foxp3 and IL-37 expression was confirmed using qRT-PCR and flow cytometry.

For human Treg cell expansion, Human peripheral blood from healthy donors was obtained from blood cones provided by Children's Hospital Blood Donor Center (Aurora, CO), and peripheral blood mononuclear cells (PBMC) were purified using Ficoll density gradient centrifugation. Further purification of PBMCs into CD4+CD25+CD127^{dim} Treg cells was done using the Human CD4+CD25+CD127^{dim} Regulatory T cell Isolation Kit (Miltenyi Biotech) according to manufacturer's instructions. After purification, 5×10⁶ cells/ml Treg cells in complete RPMI media supplemented with 1000 U/ml human IL-2 were then plated on 24-well plates coated with 1 μg/ml anti-CD3 (OKT3) and 5 μg/ml anti-CD28 (CD28.6). Over the course of 10-day cultures, each well was split once cell numbers reached 1×10⁶ cells/ml, and 50 U/ml IL-2 supplement complete media was added. At day 10, cells were counted, and FOXP3 and IL37 expression was confirmed using qRT-PCR and flow cytometry.

### IL-37 Overexpressing Vector Transduction of Expanded Human or NOD Mouse Treg Cells

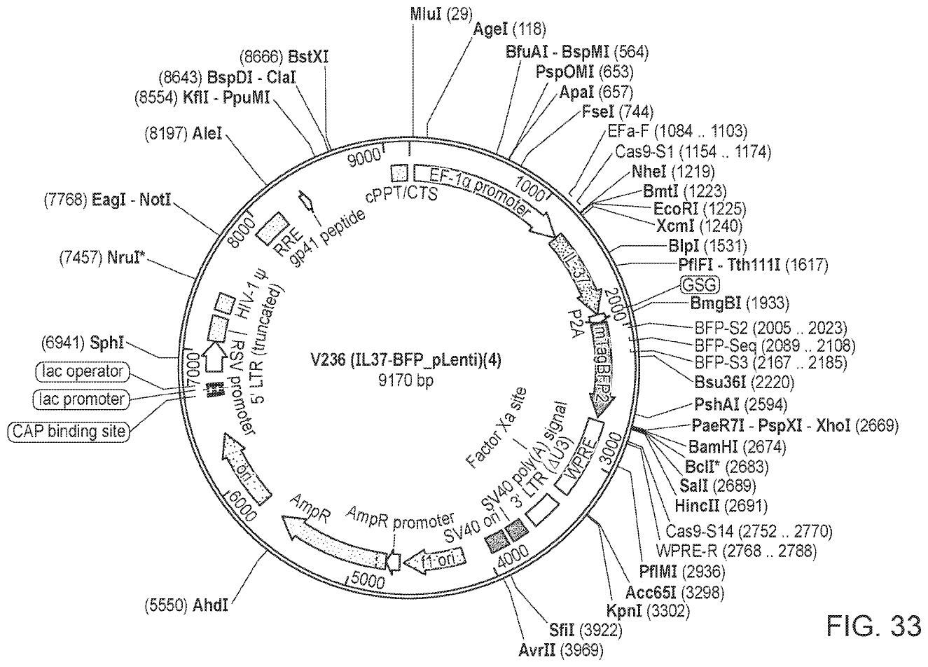

Following a 10-day expansion of either primary human or NOD mouse Treg cells, cells were then lentivirally transduced with either a control vector or an IL37 over-expressing vector, vector shown in FIG. 33. 5×10⁵ Treg cells in 500 ml complete media were plated on 20 μg/ml RetroNectin (TaKaRa, Japan) with a 4-20 μg/cm2 plate area volume. 500 ml of viral supernatant derived from lenti-IL37 OE vector transfected 293T cells were added to each well and spinfected for 90 min at 2000 rpm. Following spin, the supernatant was replaced with 100 U/ml IL-2 containing complete media and cultured until IL37 expression was confirmed by qRT-PCR before use in vitro or in vivo. Protocol was adopted from 2021 Wu et. al., A method for expansion and retroviral transduction of mouse regulatory T cells, in the Journal Immunol. Methods.

### Mouse Psoriasis Model

WT and IL37 Tg mice at 8 weeks of age were anesthetized with isoflurane and received a daily topical dose of 62.5 mg of commercially available imiquimod cream (5%) (Aldara; 3M Pharmaceuticals) on the right ear for 6 consecutive days. Control mice were treated similarly with a control vehicle cream (Vaseline Lanette cream). The psoriatic responses (erythema, scaling, and thickness of the back skin) were scored daily prior to anesthetic and topical drug treatment. An objective scoring system was developed based on the clinical Psoriasis Area and Severity Index (PASI) to score the severity of inflammation of the back skin. Erythema, scaling, and thickening was scored independently on a scale from 0 to 4:0, none; 1, slight; 2, moderate; 3, marked; 4, very marked (J Immunol 2009; 182:5836-5845). The scoring was followed for 7 days after application of the imiquimod cream, as it will be continuously increased.

### BRGS-GVHD (Graft-vs-Host Disease) Model Using Jurkat Cells

Female mice were injected intraperitoneally with a 100 μl mixture of CD25− (negative) PBMC (15×10⁶) and human E6 Jurkat leukemia cell line (1.5×10⁶) that does not produce leukemia in the mice, which will only be used in the induction/suppression of GVHD. IL37 OE vs. control Jurkat cells. Mice received 1) only CD25-PBMC or 2) CD25-PBMC plus Jurkats (either IL37 OE or control) cells was used. All PBMC and Jurkats injected into the mice have been prepared in a sterile, aseptic manner. Immunocompromised BALB/c Rag2^{-/-}Il2rg^{-/-}Sirpa^{NOD} mice (BRGS mice) were used as recipients. Mice were monitored twice a week for weight changes and other signs of GVHD (ruffled fur, reduced mobility, hunched posture, ataxia, facial edema, severe diarrhea, lethargy, inability to eat and drink, excessive pain and discomfort, reduced response to stimuli, poor skin integrity).

### BRGS-GVHD (Graft-vs-Host Disease) Model Using Primary Human Treg Cells

Female mice were injected intraperitoneally with a 100 μl mixture of CD25− (negative) PBMC (15×10⁶) and human Treg cells (15×10⁶). Control and IL37 OE human Treg cells, purified from PBMC and transfected with the IL-37 over-expressing vector. BRGS mice received 1) only CD25-PBMC or 2) CD25-PBMC plus human Treg (either control or IL37OE) cells were used. All PBMC and Treg cells injected into the mice have been prepared in a sterile, aseptic manner. Mice were monitored twice a week for weight changes and other signs of GVHD (ruffled fur, reduced mobility, hunched posture, ataxia, facial edema, severe diarrhea, lethargy, inability to eat and drink, excessive pain and discomfort, reduced response to stimuli, poor skin integrity). A GVHD clinical symptoms scoring system was used to determine when the human endpoint/euthanasia for the mice in this study. The scoring system was based on the following criteria (McDaniel et. al., 2019) (Cooke et. al., 1996).

| | Weight | Diar- | | | | Skin |
|---|---|---|---|---|---|---|
| Score | Loss | rhea | Posture | Activity | Fur | Integrity |
| 0 | 0-10% | no | normal | normal | normal | normal |
| 1 | 10-15% | yes | Hunching | Mild to | Mild to | Moderate |
| | | | at rest | moderate | moderate | scaling |
| | | | | decrease | ruffling | of paws |
| | | | | | | and tail |
| 2 | 15-20%* | | Severe | Severe | Severe | Severe |
| | | | hunching | decrease | ruffling | scaling |
| | | | | | | of paws |
| | | | | | | and tail |

### Traumatic Brain Injury (TBI) Model With Adoptive Cell Transfer of Treg Cells

Induction of TBI in C57BL/6 mice was performed according to the protocol described in 2013 Namjoshi et. al., Towards clinical management of traumatic brain injury: a review of models and mechanisms from a biomechanical perspective, in the journal of Dis. Model Mech. Following induction of TBI, mice were injected i.p. with 1×PBS (vehicle), or 2.5×10⁶ MACS purified and expanded CD4+CD25^{hi} T cells from either donor WT or IL37 Tg mice. Following 20 days after TBI induction, mice were sacrificed, and spleen and cervical lymph nodes were harvested for CD3+ T cell flow cytometry-based analyses

### Statistical Analysis

Data are expressed throughout as mean±standard error mean (SEM). Data sets were compared using the two-tailed unpaired Student's t-test. Statistical analysis (Student's t-test, ANOVAs, and column statistics) and graphing were performed using Prism 4 (GraphPad, La Jolla, CA). Differences were considered statistically significant when p<0.05.

### INTRODUCTION

The study disclosed herein used examined IL37 expression in human PBMCs. First, elevated IL-37 expression in human Treg cells was confirmed using a PrimeFlow RNA assay. Then, E6 Jurkat cells were transduced with lentivirus made from an IL-37 overexpressing vector (IL37 OE), demonstrating the Treg-like phenotype and function in stably transduced cells. Jurkat cells are suitable for developing a Treg-like cell line due to their CD4+ T cell phenotype, ease of culturing and genetic manipulation, and their ability to become suppressive. The results disclosed herein provide an alternative method for developing a stable Treg-like cell line from non-Treg CD4+ T cells for therapeutic use.

### IL-37 Expression in Regulatory T Cells

### IL37 Expression is Elevated in Treg Cells

A flow cytometry-based assay was used to measure IL37 mRNA levels in freshly obtained human PBMCs without culture or fractionation. Cells were stimulated with LPS similar to the condition from other studies to induce IL-37. FIGS. 1A-1B show flow cytometry analysis of cell surface proteins and IL37 mRNA from immune cells. The gating strategy in FIG. 1A (myeloid cells) and FIG. 1B (lymphoid cells) was performed to measure the percent and quantity of IL37 in multiple immune cell types. The average percentage of cellular subsets among the total IL37 mRNA-expressing cells from 5 healthy human donors is shown in FIG. 1C. Peripheral blood mononuclear cells (PBMCs) were treated in the absence (Unstim) or presence of 100 ng/ml LPS (+LPS) for 24 hours. As shown in FIG. 1C, CD3+ T cells (48.7%) and monocytes (43.5%) comprised the largest proportion of IL37 mRNA-expressing immune cells, whereas other cells comprised less than 15% of IL37 mRNA-expressing cells. Treatment with LPS slightly increased the contribution by T cells (to 52.2%) and slightly decreased the contribution by monocytes to 39.2%.

Each cell type was then analyzed for IL37 expression to determine the frequency of IL37 mRNA-expressing cells in each myeloid and lymphoid cell subsets. Cells were treated in the absence (−) or presence (+) of 100 ng/ml LPS for 24 hours. As shown in FIG. 1D, about half of CD3+ T cells (48%) and monocytes (44.7%) expressed IL37 mRNA, whereas IL37 was expressed in less than 5% of other populations (dendritic cells, B cells, and NK cells). Among T cells, Treg cells (CD4+CD25hiCD45RBlo) (green) contained the highest proportion of IL37+ cells (73%), and this frequency was increased further from 73% to 88% after LPS stimulation in Treg cells (p<0.05 via Student's t test).

IL37 expression levels in each cell type was further analyzed. As shown in FIGS. 1E-F, the IL37 mRNA level was highest in monocytes (black squares) compared to other immune subsets, and the level was further increased after LPS stimulation. Treg cells (green) expressed the highest IL37 level among lymphoid subsets, which was further increased after LPS stimulation (p<0.001 via Student's t test). These results indicate that IL-37 is highly expressed in human Treg cells. Considering its role in inducing FOXP3, IL-37 was identified as a good candidate for establishing a Treg-like cell line.

### Construction of a Treg-Like Cell Line

### Overexpression of IL-37 Leads to the Development of a Treg Phenotype in Jurkat Cells

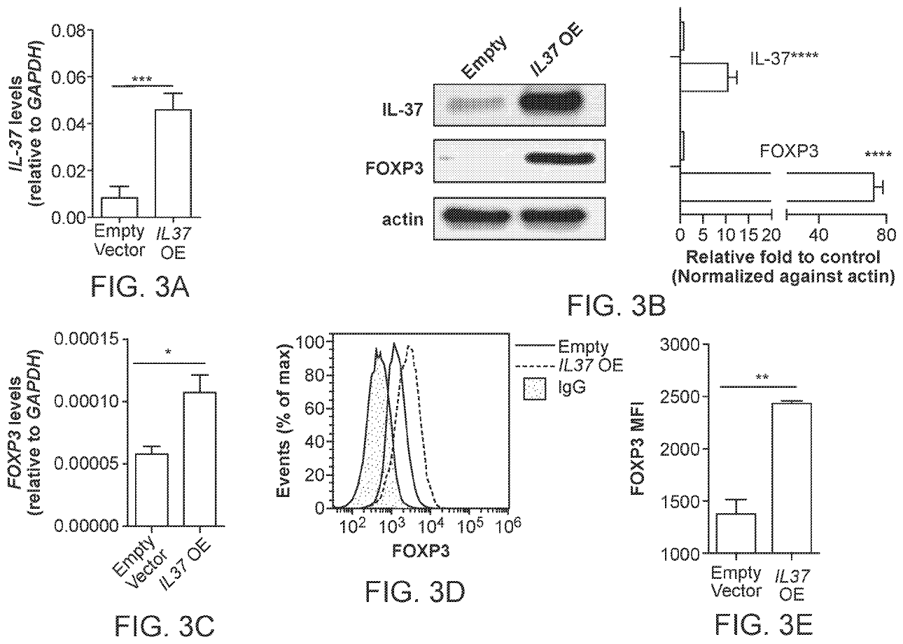

To develop a stable Treg cell line, human leukemia E6 Jurkat T cells were transduced with a lentiviral FLAG-tag IL37 overexpressing vector. A schematic of the IL37 expression vector is depicted in FIG. 2. To measure IL37 mRNA, qRT-PCR using the same primer sets used for the PrimeFlow® RNA assay was employed. As a control, Jurkat cells were transduced with an empty vector control. While empty vector/control Jurkat cells expressed very little IL-37 at both the gene and protein levels, FLAG-tag IL37 transduced cells overexpressed IL-37 at the gene (FIG. 3A) and protein levels (FIG. 3B), confirming successful transduction and translation, respectively. As shown in FIG. 3A, IL37 mRNA expression in Jurkat cells containing the expression vector was about 9-fold higher than Jurkat cells containing the empty vector control (p<0.001, by Student's t test). Expression of GAPDH served as internal expression control. As shown in FIG. 3B, IL-37 protein level was about 9-fold higher in Jurkat cells containing overexpression vector (IL37 OE) compared to Jurkat cells containing empty vector control. Protein levels were quantified and normalized against actin as a control.

As shown in FIG. 3C, IL-37 overexpressing (IL37 OE) Jurkat cells had elevated FOXP3 gene expression (˜5-fold increase) compared to control Jurkat cells. FOXP3 levels were normalized relative to GAPDH as a control. This was further confirmed at the protein level by western blotting (FIG. 3B) and by flow cytometry (FIGS. 3D-E). Specifically, FOXP3 densitometry measurements showed a 78-fold increase in FOXP3 protein expression in IL37 OE Jurkat cells, as shown in FIG. 3B.

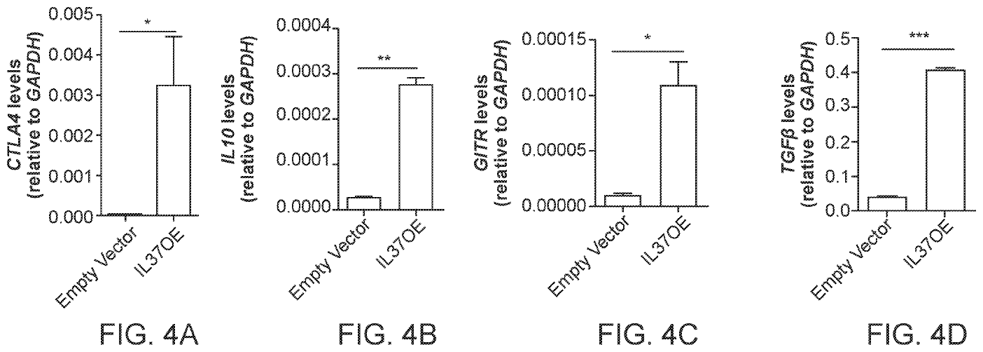

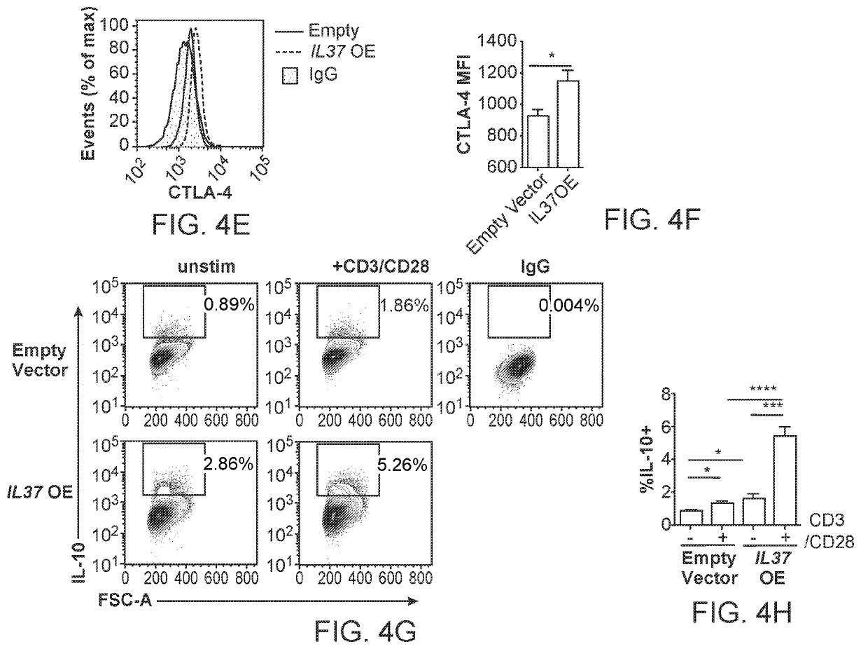

Along with elevated FOXP3, IL37 OE Jurkat cells showed elevated gene expression of cytotoxic lymphocyte antigen-4 (CTLA-4), IL-10, glucocorticoid-induced TNFR-related (GITR), and transforming growth factor-β (TGF-β), as shown in FIGS. 4A-D. These proteins are markers commonly found on highly suppressive Treg cells, suggesting that the IL37 OE Jurkat cells may present a similar phenotype. CTLA-4 protein expression was evaluated by flow cytometry. As shown in FIGS. 4E-F, CTLA4 expression was higher in IL37 OE Jurkat cells. FIG. 4F shows that CTLA-4 protein expression in IL37 OE Jurkat cells was about 1.3-fold higher than empty vector control cells (p<0.05 via Student's t test). Furthermore, as shown in FIG. 4G, a significant proportion of IL37 OE Jurkat cells expressed IL-10 protein without stimulation (2.86%), which was further increased with CD3/CD28 stimulation (5.26%), compared to control Jurkat cells (0.89% without stimulation, and 1.86% with stimulation). The percentage of IL-10+ cells was quantified and is shown in FIG. 4H. IL37 OE Jurkat cells that were stimulated showed about 2.5-fold increase in % IL-10+ over unstimulated IL37 OE Jurkat cells (p<0.001 via Student's t test), and stimulated vector control cells (p<0.0001 via Students t test).

These data confirm that IL-37 overexpression in Jurkat cells induced a Treg cell phenotype. It is known that both CTLA-4 and IL-10 play a significant role in suppressing an adaptive immune response, suggesting that IL-37 overexpression in Jurkat cells also leads to the induction of a Treg functionality.

### IL37OE Jurkat Cells Suppress Naïve CD4+ T Cell Proliferation Comparable to Human Primary Treg Cells

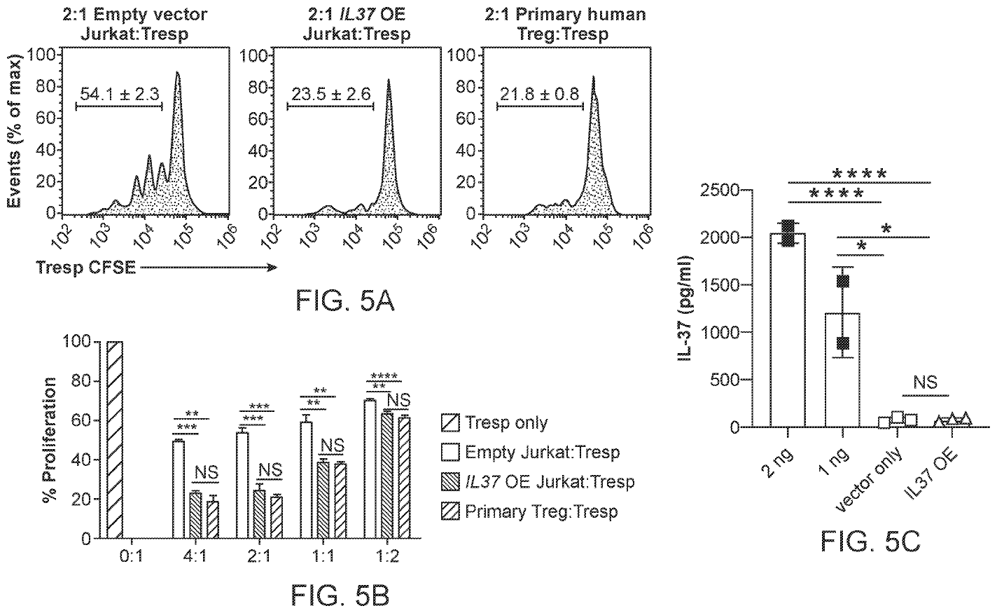

FOXP3 expression is integral in Treg suppressive function. Therefore, IL37 OE Jurkat cells were evaluated for their suppressive function in vitro. Freshly isolated human CD4+CD25lo T cell responder (Tresp) cells were labeled with CFSE and co-cultured in CD3-stimulatory conditions for 5 days with either control Jurkat cells or IL37 OE Jurkat cells. As shown in FIG. 5A, the co-culture of IL37 OE Jurkat cells with Tresp cells at a ratio of 2:1 significantly suppressed proliferation of Tresp cells (˜50% reduction compared to control Jurkat cells), comparable to human primary Treg cells (FIG. 5A). No significant difference was found in suppressive ability between IL37 OE Jurkat cells and human primary Treg cells across all Treg/Tresp ratios.

As shown in FIG. 5B, IL37 OE Jurkat cells were cultured with Tresp at ratios ranging between 0:1, 4:1, 2:1, 1:1, 1:2, 1:10 for 5 days, and the percentage of proliferation was assessed at each ratio and compared to Tresp cells cultured with vector control Jurkat cells. IL37 OE Jurkat cells reduced proliferation of Tresp cells compared to vector control at 4:1, 2:1, 1:1 and 1:2 ratio. The amount of suppressive activity correlated with the Jurkat: Tresp ratio, with a higher number of Jurkat cells resulting in more suppressive activity. The highest suppressive activity was seen at 4:1 and 2:1, each showing about 50% proliferation relative to vector control (p<0.001 via Student's t test), and 25% proliferation relative to the absence of Jurkat cells.

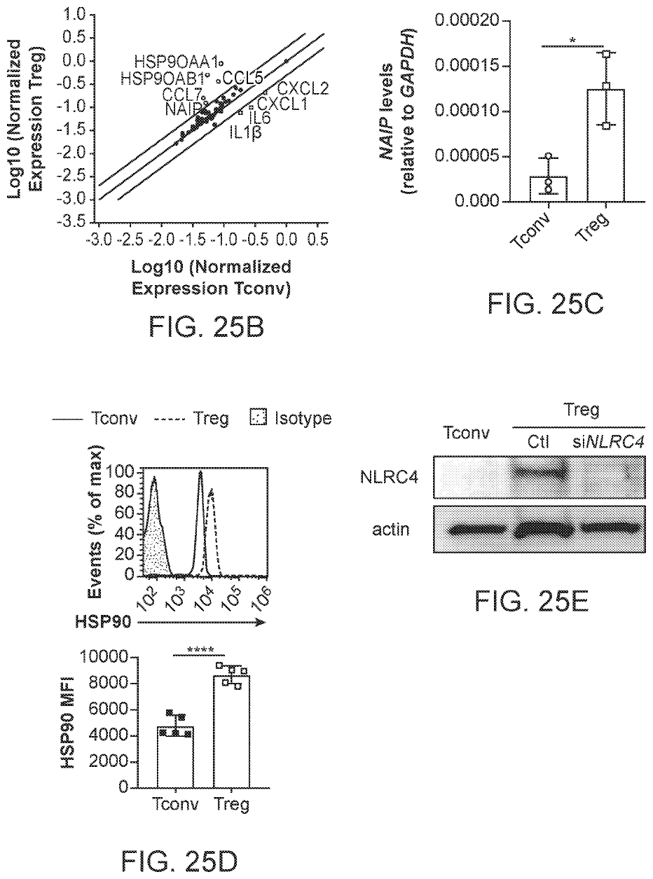

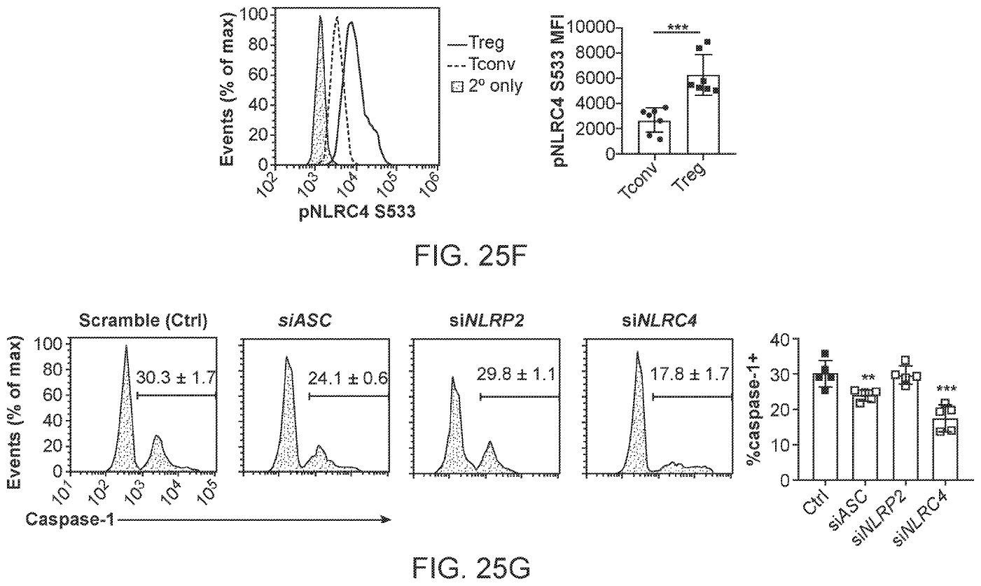

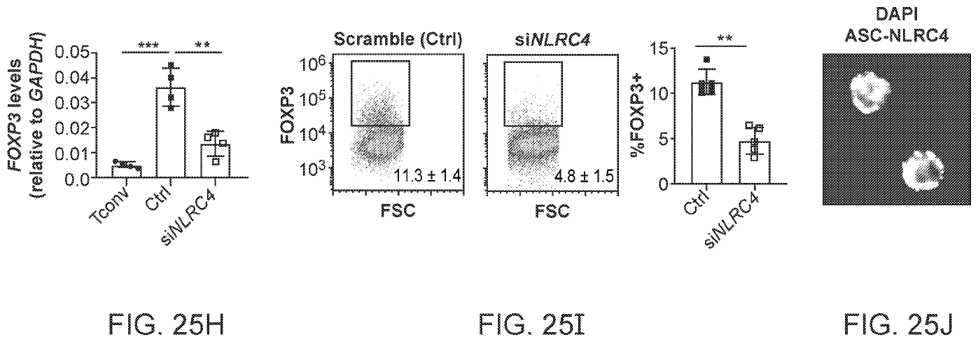

Because secreted/extracellular IL-37 has been shown to inhibit cell activation, it was examined to determine if IL-37 was released from IL37 OE Jurkat cells to suppress Tresp cells. IL-37 ELISA showed that the secretion of IL-37 from IL37 OE Jurkat cells was minimal and compatible with the level of IL-37 secretion from the vector control (FIG. 25C), suggesting that intracellular IL-37 expression, but not extracellular IL-37 secretion, was responsible for the suppressive function of IL37 OE Jurkat cells.

IL37 OE Jurkat Cells Suppress Xenogeneic Graft-Versus-Host Disease (xenoGVHD)

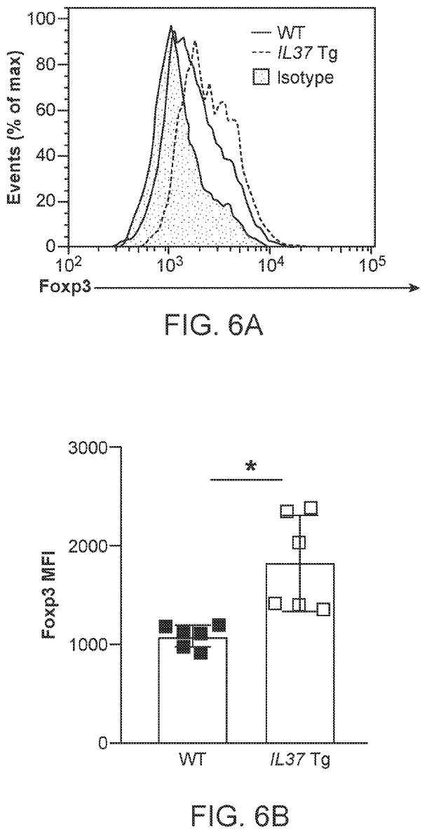

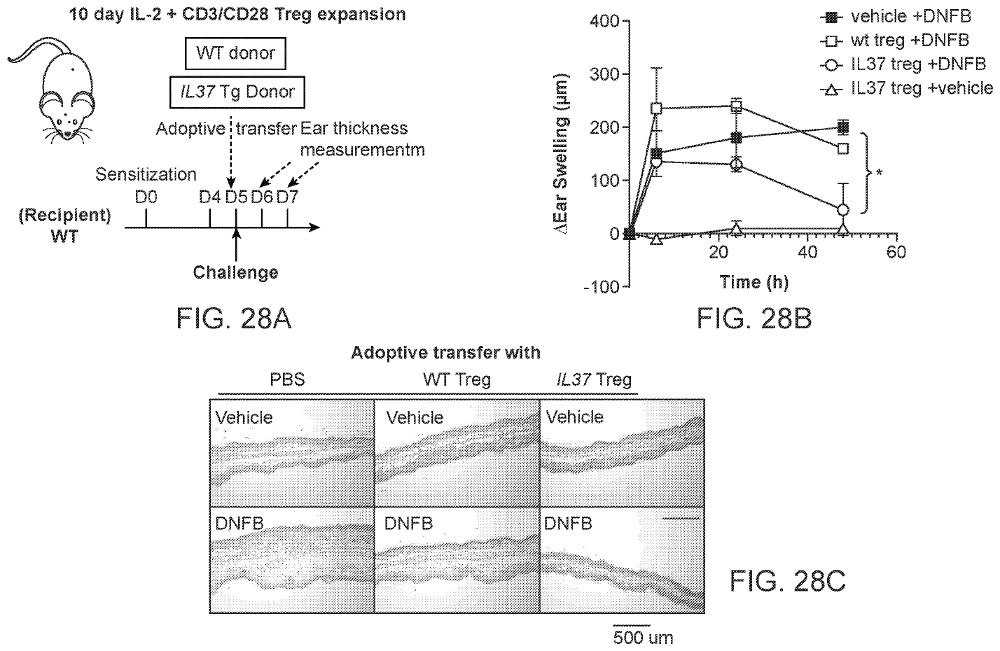

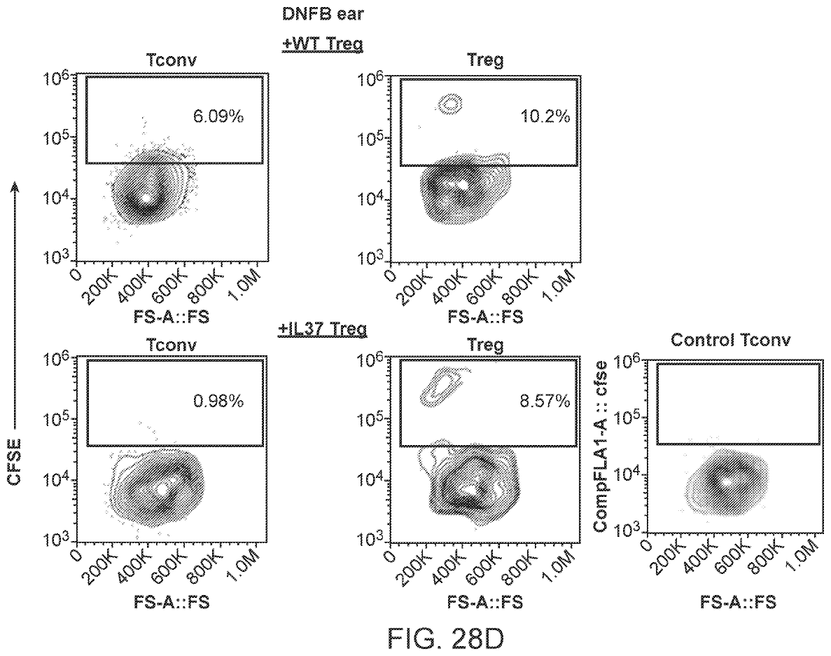

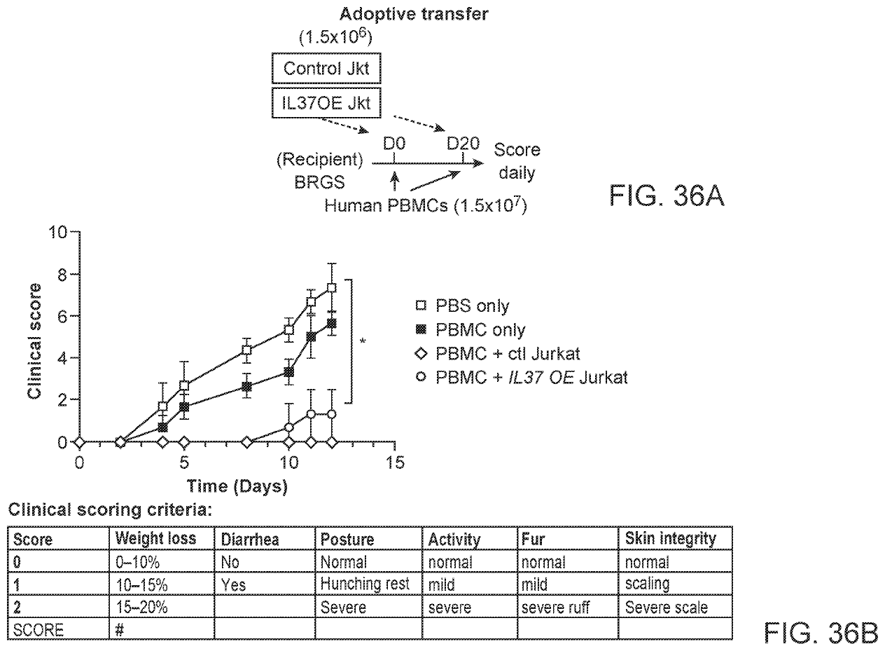

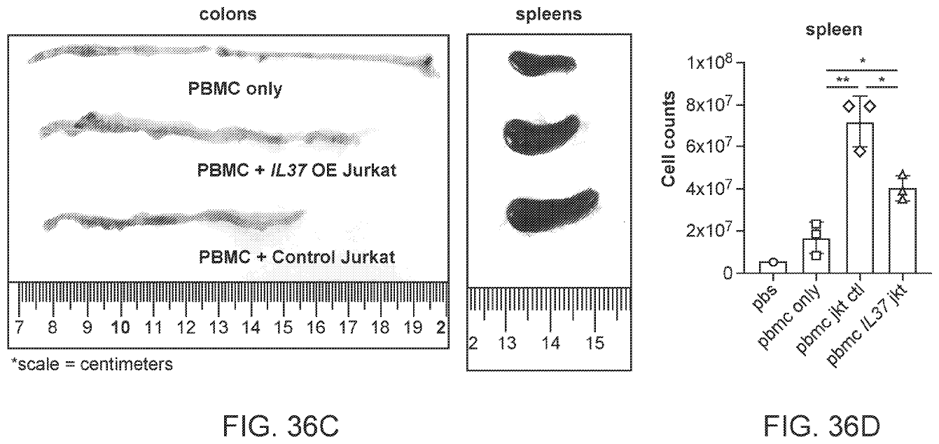

To study the suppressive function of IL37 OE Jurkat cells (human cells) in vivo, a “humanized mouse model of xenogeneic graft-versus-host disease (xenoGVHD)” was used. In this model, human PBMCs were adoptively transferred into immunocompromised BALB/c Rag2^{-/-}Il2rg^{-/-}Sirpa^{NOD} (BRGS) mice lacking lymphocytes (T, B, and NK cells) and SIRPA that recognizes human CD47-Fc, allowing efficient engraftment of human PBMC to induce xenogeneic reaction (FIG. 36A). Mice were co-transferred with either PBS, control Jurkat or IL37 OE Jurkat cells. The xenoGVHD responses were ameliorated by co-transferring IL37 OE Jurkat cells, whereas control Jurkat cells could not prevent xenoGVHD (FIG. 36B). Using the scoring criteria shown in FIG. 36, it was found that 12 days after PBMC injection, mice receiving IL37 OE Jurkat cells had significantly lower clinical scores than either mice that received PBMC only or PBMC+control Jurkat cells (FIG. 36B). FIG. 36C shows images of colons (left) and spleens (right) from mice adoptively transferred with human PBMCs and treated with either PBS, control Jurkat cells, or IL37 OE Jurkat cells, displaying increased inflammation and shortening of colon (left) and increased spleen size (right) in mice treated with control Jurkat cells compared to the other mice (FIG. 6C). Likewise, the analysis of mouse spleen revealed the massive infiltration of human CD3+T, CD8+T, and CD4+ T cells in the mice that received human PBMC and control Jurkat cells, whereas human T cell infiltration was significantly reduced in the mice that received human PBMC and IL37-expressing Jurkat cells (FIG. 28C). These findings demonstrate a clear difference between IL37 OE Jurkat cells and control Jurkat cells to suppress GVHD responses. The findings also prove that non-Treg CD4+ T cells can be reprogrammed to possess Treg cell-like functions to suppress severe immune responses such as GVHD in vivo.

### Discussion

In this study, a flow cytometry-based gene expression assay was used to examine the expression of anti-inflammatory cytokine IL-37 in human PBMCs. Results disclosed herein confirm results from an earlier study showing that human Treg cells expressed an elevated level of the anti-inflammatory cytokine IL-37. These studies revealed that Treg cells were the only lymphocyte subset to ubiquitously express high IL37 mRNA and IL-37 protein even without stimulation, suggesting an essential role of IL-37 in the Treg function and FOXP3 expression. Furthermore, the results herein demonstrate that the stable overexpression of IL-37 induced Treg-like phenotype (elevated FOXP3, CTLA-4, IL-10, and other Treg markers) and suppressive function both in vitro and in vivo in a non-Treg CD4+ T cell line, Jurkat cells.

IL-37 has been extensively studied in innate immunity and inflammatory conditions. However, its role in adaptive immunity remains less understood. While many studies reported the tolerogenic role of IL-37 in dendritic cells, only a handful of studies have investigated IL-37 in lymphocytes. A possible explanation for the limited studies in lymphocyte subsets could be the scarcity of information about IL-37's expression in lymphocytes. This study unveils that CD3+ T cells and monocytes comprise the majority of IL37 mRNA-expressing immune cells (48.7% and 43.5%, respectively). These findings contradict Rudloff et. al., who reported that IL-37 is predominantly expressed by monocytes (91% of the IL-37+ population), whereas the remaining 9% comprised T cells. They also reported that less than 0.5% of T cells expressed IL-37 protein, whereas this study shows that about half of CD3+ T cells (48%) expressed IL37 mRNA.

The discrepancies between Rudloff's study and this study could be due to the different assays used to measure IL-37. For example, Rudloff et. al. measured IL-37 protein expression using IL-37 monoclonal antibody (37D12), whereas IL37 mRNA expression was measured in this study using an IL37 mRNA specific probe and in situ hybridization that is amplified to ensure sufficient detection. While different experiments cannot be directly compared, some of the current results using an IL37 mRNA probe are consistent with previous results obtained using the IL-37 monoclonal antibody (37D12). Previous flow cytometry analysis using the 37D12 showed the IL-37 protein expression in 69% of unstimulated human Treg cells. This finding is consistent with current data using a flow cytometry-based IL37 gene expression assay where 73% of unstimulated human Treg cells expressed IL37 mRNA. Therefore, other factors might also be involved in the discrepancies, including IL-37 secretion amounts, fixation and permeabilization methodology of cells, and PBMC preparation methods.

The present study confirmed previous findings that IL-37 was elevated in human Treg cells. Treg cells are essential in peripheral tolerance to suppress and prevent an auto-reactive immune response. Stable expression of FOXP3 in Treg cells is required for their suppressive capabilities and stability and is critical in maintaining peripheral tolerance. A recent paper showed that FOXP3 expression was decreased by IL37 knockdown in primary human Treg cells. Since IL-37 was found to interact with the FOXP3 transcription factor SMAD3, IL-37 could contribute to FOXP3 gene expression via SMAD3. Disclosed herein are results showing that Jurkat cells express almost undetectable levels of IL-37, which could explain why FOXP3 is not expressed in Jurkat cells but is induced by overexpressing IL-37. SMAD3 was shown to repress IFN-g and inhibit T-BET, the Th1 transcription factor expressed in Jurkat cells. Therefore, IL-37 expression in Jurkat cells might alter the T-cell differentiation balance through Th1/Treg cell transcription factors via SMAD3. Further analyses are needed to compare IL-37OE Jurkat cells to primary human Treg cells and examine signaling pathways to maintain the Treg phenotype and function.

Understanding the mechanism of Treg plasticity and how Treg cells can lose FOXP3 and convert to a pro-inflammatory CD4+ T cell subset is crucial to the development of successful immunotherapies, such as Treg adoptive transfer therapy. The creation of a stable Treg cell line, such as with our IL37OE Jurkat cells, will provide a convenient model for analyzing Treg plasticity, helping to understand the molecular mechanisms that support FOXP3 expression, and aiding in the development of therapeutics for autoimmunity, cancer, and chronic inflammatory conditions. Further, the data presented herein provide evidence that non-Treg human CD4+ T cells can be reprogrammed to immunosuppressive Treg cell-like cells through overexpression of IL-37. This opens up a tremendous opportunity to control immune responses and chronic inflammatory conditions without the need for isolating and purifying a minor CD4+ T cell population.

### Conclusion

Results from this study confirm IL-37 expression in human Treg cells. E6 Jurkat cells were then transduced with lentivirus made from an IL-37 overexpressing vector (IL37 OE) and their Treg-like phenotype and function demonstrated. Jurkat cells are suitable for developing a Treg-like cell line due to their CD4+ T cell phenotype, ease of culturing and genetic manipulation, and their ability to become suppressive. The results described herein provide an alternative mechanism for developing a stable Treg-like cell line that can be used not only for studying Treg cell biology and function but also for translational application to treat autoimmunity, immune responses, and chronic inflammatory conditions.

Together, these data confirm that stable IL-37 expression in Jurkat cells induced a Treg phenotype and function.

### Example 2: IL-37 Expression in Mouse Regulatory T Cells Maintain FOXP3 Expression and Support Treg Cell Stability

While IL-37 is not present in mice, like other IL-1 family members, IL-37 shows no species specificity, and IL37 Tg mice exhibit a phenotype that mimics human IL-37 in human cells. Thus wild-type (WT) and IL37 Tg mice were used to study the functionality of IL-37 in Treg cells.

### IL-37 Expression in Mouse Treg Cells Increases FOXP3 Expression

IL-37 Transgenic (Tg)-Treg cells are more suppressive than WT-Treg cells in vitro. T cell development was examined and found that the absolute numbers of thymocytes and T cells in the thymus and spleen were similar between WT and IL37 Tg mice. However, Treg cells (CD4+CD25^{hi} T cells isolated using a Miltenyi Treg isolation kit) from the spleens of IL37 Tg mice (IL37 Tg-Treg cells) expressed a higher FOXP3 protein than those from WT mice (WT-Treg cells). As shown in FIGS. 6A-B, FOXP3 expression was about two-fold higher in IL37 Tg Treg cells compared to WT Treg cells (p<0.05).

### IL-37 Expression in Mouse Treg Cells Enhances Treg Cell Suppressive Ability In Vitro

An in vitro T cell suppression assay was used to compare function of Treg cells isolated from WT and IL37 transgenic (Tg) mice. T cell responder (Tresp) cells (CD4+CD25−) were isolated from WT mice and labelled with CFSE. CFSE-labelled Tresp cells were cultured with anti-CD3 and then co-incubated with Treg cells either from WT or IL37 Tg mice for 3 days at ratios of 1:1, 1:2, 1:3, 1:8 and 1:12 (Treg:Tresp). Tresp cells were also incubated in the absence of Treg cells, and either with (“Tresp only”) or without (naïve Tresp”) CD3 to serve as positive and negative controls, respectively.

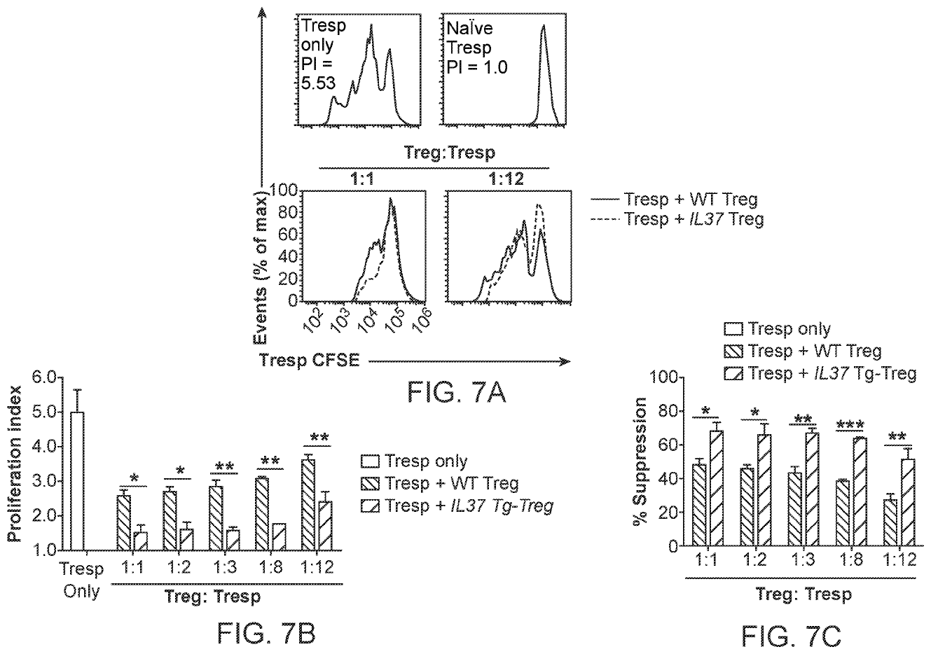

Both WT- and IL37 Tg-Treg cells were functionally suppressive in vitro and inhibited the proliferation of naïve CD4+CD25− T responder (Tresp) cells isolated from WT mice; however, Tresp cell proliferation was suppressed more by IL37 Tg-Treg cells than WT-Treg cells, as shown in FIGS. 7A-C. As shown in FIG. 7C, the suppression of Tresp cells increased as the ratio Treg cells to Tresp cell (Treg:Tresp) increased. A ratio of Treg to Tresp of 1:1 to 1:8 showed a percent suppression of Tresp cells between 40-45%. The suppression at a 1:12 ratio was about 25%.

### IL-37 Tg-Treg Cells Stabilize FOXP3 Expression and Prevent Mouse Treg Cell Conversion to Tconv Cells in Inflammatory Conditions

Under inflammatory or pathogenic circumstances, some Treg cells lose FOXP3 expression and/or their suppressive function. IL-6 is known to downregulate FOXP3 expression by activating STAT3 and counteracting STAT5 signaling in Treg cells. Treg cells isolated from WT and IL37 Tg mice were cultured for 48 hours with anti-CD3/CD28 in the absence or presence of mouse IL-6 (20 ng/ml) and FOXP3 expression was analyzed in each cell via qRT-PCR. GAPDH expression was also measured and served as a control.

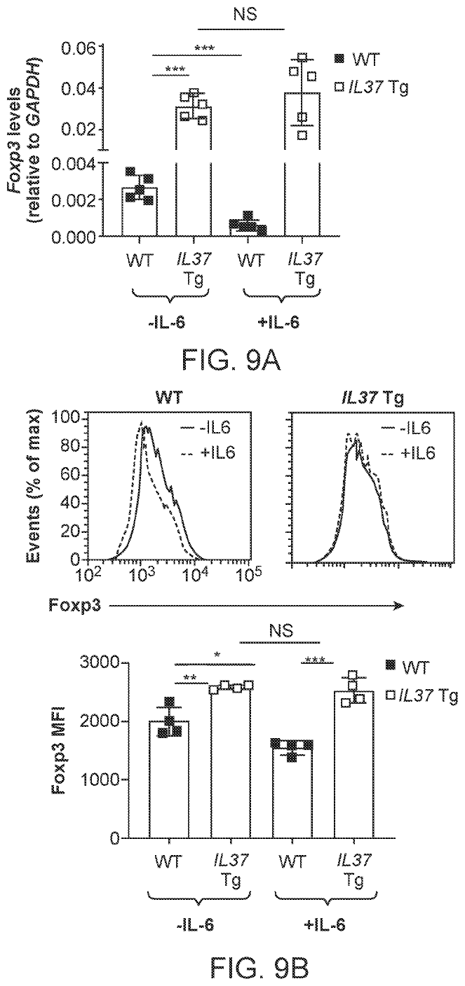

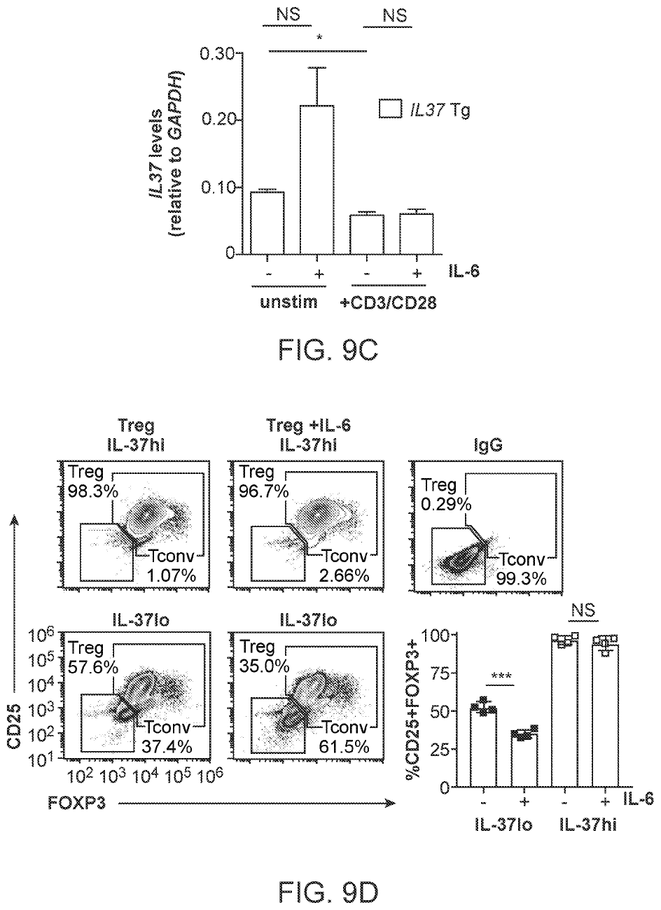

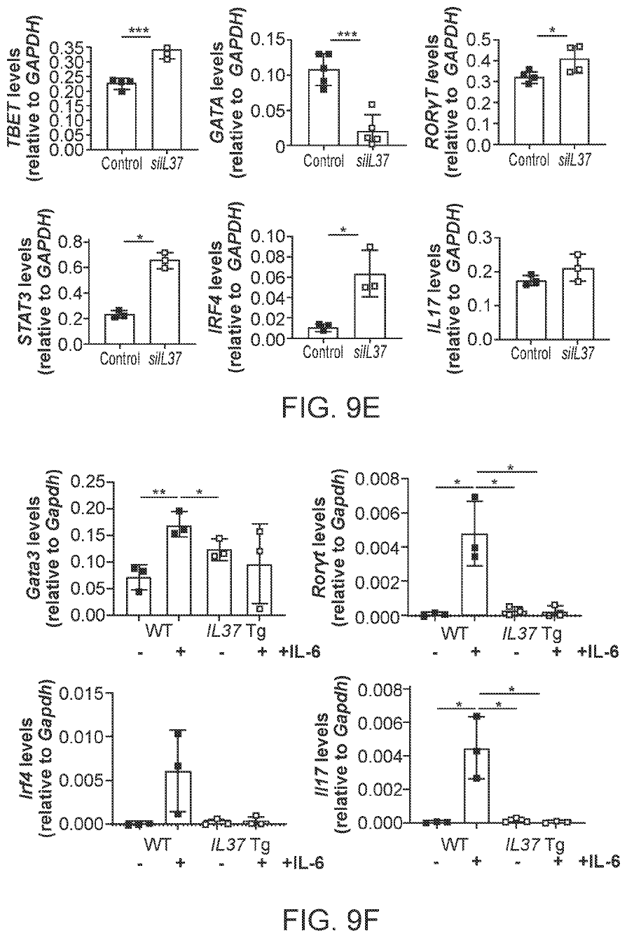

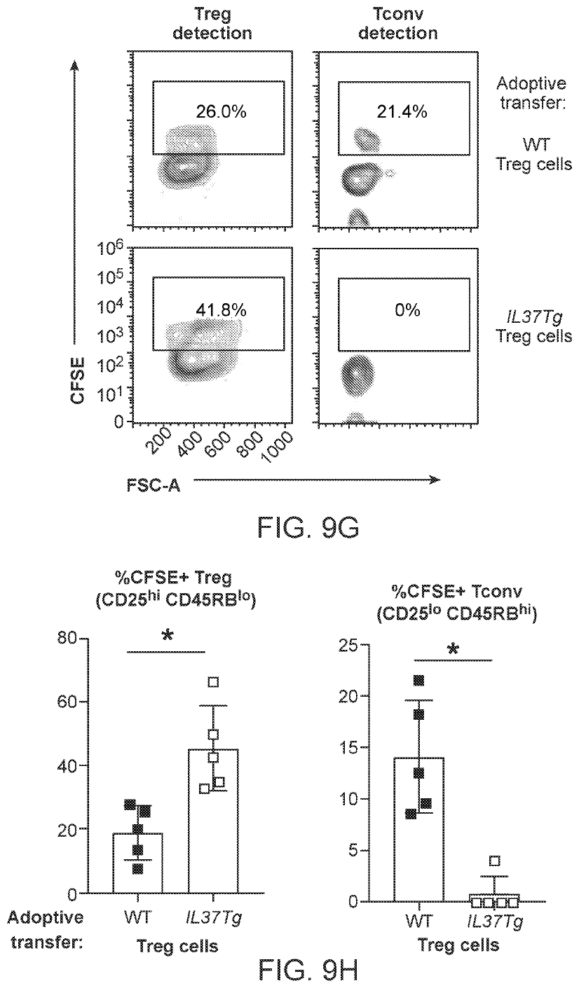

As shown in FIG. 9A the expression of FOXP3 in Treg cells from IL37 Tg mice was significantly higher than in Treg cells from WT mice in the presence (about 80-fold) and absence (ten-fold) of IL-6. The addition of IL-6 to the WT Treg cells reduced mRNA and protein levels of Foxp3 upon stimulation with anti-TCR/CD28. However, IL-6 did not reduce Foxp3 in IL37 Tg-Treg cells, even when they were cultured in the absence of anti-TCR/CD28. Of note, IL-37 levels were not decreased by IL-6 in IL37 Tg-Treg cells. These results demonstrate that the transgenic expression of IL-37 in mouse Treg cells prevents inflammation-induced Foxp3 reduction at the mRNA and protein levels in vitro, leading to Foxp3 stability.

Adoptively transferred Treg cells were labelled with carboxyfluorescein succinimidyl ester (CFSE) to trace them and monitor their expansion in vivo. CHS skin analysis of the mice that received WT Treg cells revealed an appearance of CFSE+ Tconv cells and a reduction in CFSE+ Treg cell frequency, suggesting reprogramming of WT Treg cells to Tconv cells in vivo, as shown in FIG. 9B (upper panels) and FIG. 9C. In contrast, IL37 Tg-Treg cells were not converted to Tconv cells in the inflamed skin after the transfer, as shown in FIG. 9B (lower panels) and FIG. 9C.

Together, these data demonstrate that the transgenic expression of IL-37 in mouse Treg cells prevents Foxp3 reduction and Treg cells' conversion to Tconv cells in vivo, leading to the suppression of T cell infiltrates at local inflammatory sites.

### Example 3: Transgenic Regulatory T Cells are Highly Suppressive In Vivo

### IL-37 Transgenic (Tg) Regulatory T Cells Suppress Skin Inflammation

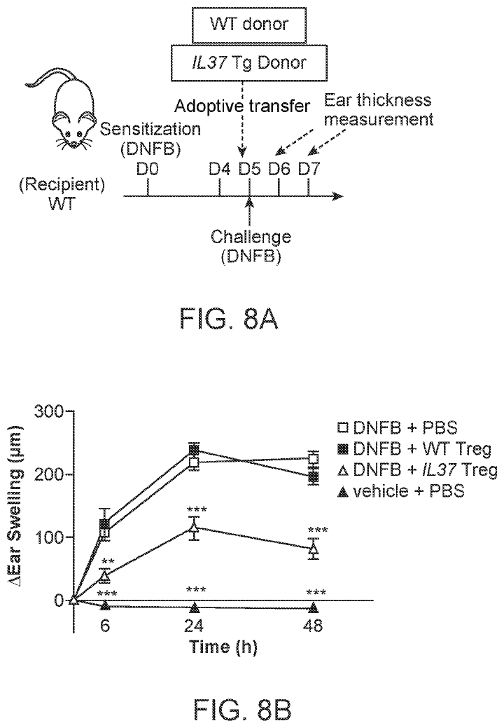

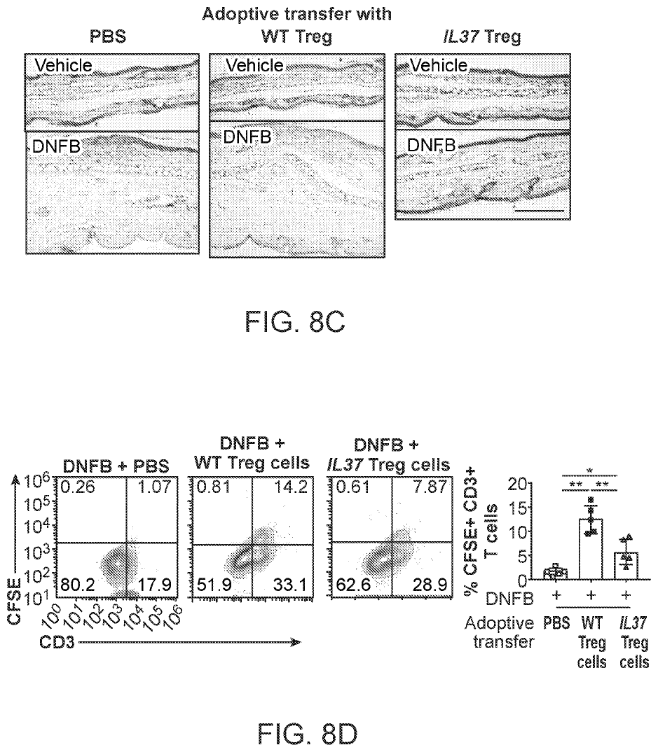

The suppressive activity of modified regulatory T cells expressing IL-37 was analyzed in vivo using a contact hypersensitivity (CHS) model. A schematic of the CHS model used is depicted in FIG. 8A. Specifically, in this model, hapten DNFB (0.5%)-sensitized WT mice were adoptively transferred with 5×10⁵ magnetic activated cell sorting (MACS)-purified WT- or IL37 transgenic (Tg)-Treg cells via tail vein injection. Mice were subsequently challenged with 0.2% DNFB. Hapten DNFB sensitization was designated at day 0 (DO), and the subsequent challenge occurred on Day 5 (D5). Ear thickness measurements were conducted on ear specimens taken on day 6 and day 7. The CHS responses in sensitized WT mice after DNFB challenge were ameliorated by the adoptive transfer of IL37 Tg-Treg cells. This reduction was not observed in mice receiving WT-Treg cells, which showed swelling levels comparable to PBS controls. As shown in FIG. 8B, the ear swelling in WT mice that received adoptive transfer of IL-37 Tg-Treg cells was significantly reduced after 6 hours, 24 hours and 48 hours post-challenge compared to mice receiving WT Treg cells and PBS control. The largest reduction was seen after 6 hours post-challenge (˜60-70% reduction in swelling), while the reduction in swelling after 24 and 48 hours was ˜50%. As shown in FIG. 8C, the ear tissues showed a reduction in skin hyperplasia, dermal edema, and inflammatory infiltrates in mice receiving IL37 Tg-Treg cells but not those receiving WT-Treg cells. Together, the data demonstrate that IL37 Tg-Treg cells are highly suppressive in vivo compared to WT-Treg cells.

### Conclusion

In this study, a flow cytometry-based gene expression assay was used to examine the expression of anti-inflammatory cytokine IL-37 in human peripheral blood mononuclear cells (PBMCs). It was shown that elevated IL-37 mRNA and protein expression occurs in human Treg cells, even without stimulation. A non-Treg CD4+ T cell line, E6 Jurkat cells, was transduced with lentivirus made from an IL-37 overexpressing vector (IL37 OE) and these cells exhibited a Treg-like phenotype (elevated FOXP3, CTLA-4, IL-10, and other Treg markers) and suppressive function. These results are suggestive of an essential role of IL-37 in Treg function and FOXP3 expression and show that a non-Treg cell line can be shifted toward a more Treg-like functional capacity.

As indicated in the present disclosure, Jurkat cells are suitable for the development of a Treg-like cell line due to their CD4+ T cell phenotype, ease of culturing and genetic manipulation, and their ability to become suppressive. Overexpression of IL37 in non-Treg Jurkat cells provide an alternative mechanism for developing a stable suppressive Treg-like cell line for use in the treatment of autoimmunity and cancer.

Together, these data demonstrate that IL-37 expression causes these transgenic regulatory T cells to become highly suppressive in vivo compared to wildtype regulatory T cells.

### Example 4: Mouse Regulatory T Cell FOXP3 Expression and Stability Requires Caspase-1

### Introduction

Regulatory T (Treg) cells play a vital role in suppressing immune responses, inducing tolerance and maintaining homeostasis (Josefowicz et. al., 2012). The transcription factor forkhead box P3 (FOXP3) expression is key to the suppressive function of Treg cells (Overacre and Vignali, 2016), and its mutations or deletion leads to a loss of functional Treg cell populations and subsequent development of severe autoimmunity and/or inflammation in both humans and mice (Chinen et. al., 2016; Hori, 2014; Huehn et. al., 2009). Studies have shown that sustained FOXP3 expression and subsequent Treg cell stability in the periphery are maintained by the transcriptional and epigenetic control of FOXP3 in its promoter and conserved non-coding DNA sequences as well as posttranslational modifications (Yue et. al., 2016). Three signaling pathways, including T-cell receptor (TCR), IL-2, and TGF-β signaling, as well as FOXP3 expression itself, induce and maintain FOXP3 expression in Treg cells during thymic development and adaptive immune insults (Chinen et. al., 2016; M. O. Li and Rudensky, 2016; Sakaguchi et. al., 2013). TCR signaling regulates the activity of a transcription factor NFAT that directly induces FOXP3 expression (Bennett et. al., 2001; Shevach et. al., 2001). IL-2 and TGF-β activate other transcription factors STAT5 and Smad3, respectively, inducing FOXP3 expression (Overacre and Vignali, 2016).

Despite their critical roles in the induction and maintenance of FOXP3 expression in the thymus and during activation, the roles of these signaling pathways (TCR, IL-2, and TGF-β signaling) remain elusive in a steady-state where the involvement of TCR and cytokines is limited (Hori, 2014). For example, continued FOXP3 expression in Treg cells is TCR-independent during the resting state, and TCR signaling lowers FOXP3 expression rather than increasing it when IL-2 is low (M. O. Li and Rudensky, 2016). TGF-β-induced FOXP3 expression is also dispensable in Treg cells in vitro and in vivo (Polansky et. al., 2008; Schlenner et. al., 2012). Nonetheless, FOXP3 is still highly expressed and functional in Treg cells during the resting state without IL-2 (Chinen et. al., 2016; Rubtsov et. al., 2010). These findings suggest that alternative mechanisms and/or molecules maintain FOXP3 expression in Treg cells, thus preserving immune tolerance.

Here, proteins that interacted with three FOXP3-regulating transcription factors, NFAT, STAT5, and Smad3 were screened, and identified the anti-inflammatory cytokine IL-37 as a Smad3 interacting intracellular signaling molecule that is integral in FOXP3 expression in human Treg cells. As one of eleven IL-1 family members, IL-37 is the only known member to be broadly anti-inflammatory (Dinarello et. al., 2016; Garlanda et. al., 2013). Using human blood cells and a genetically engineered mouse model expressing human IL-37 (IL37 Tg mice), it was demonstrated that Treg cells require IL-37 for their maintenance of FOXP3 expression and suppressive function not only in a steady-state to support Treg cell homeostasis but also in an inflammatory environment, thereby maintaining Treg cell stability and preventing their conversion to conventional T (Tconv) cells in vitro and in vivo. Using gene expression array, pharmacological and genetic inhibition, as well as another genetically engineered mouse model expressing mutant human IL-37 (IL37D204 Tg (D20A) mice) provides evidence that human Treg cells utilize the NAIP/NLRC4 inflammasome complex to cleave/activate caspase-1 and activate IL-37, which then interacts with Smad3 and translocates into the nucleus to induce FOXP3 transcription subsequently. These results described herein reveal a role for nuclear IL-37 in human Treg cells, providing insight into the mechanisms regulating FOXP3 expression and Treg cell stability and underscoring the connection between the inflammasome and Treg cell function in both steady-state and inflammatory conditions.

### I. Preliminary Data

### Binding Partner Screening of FOXP3-Regulating Transcription Factors Identifies IL-37 as a Pivotal Molecule to Maintain FOXP3 in Human Treg Cells

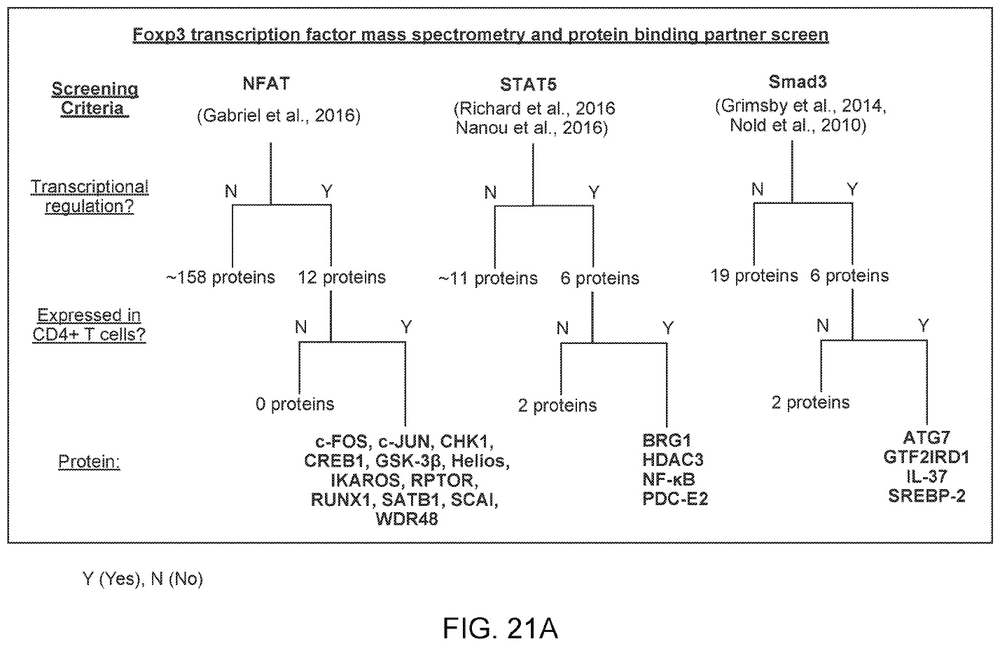

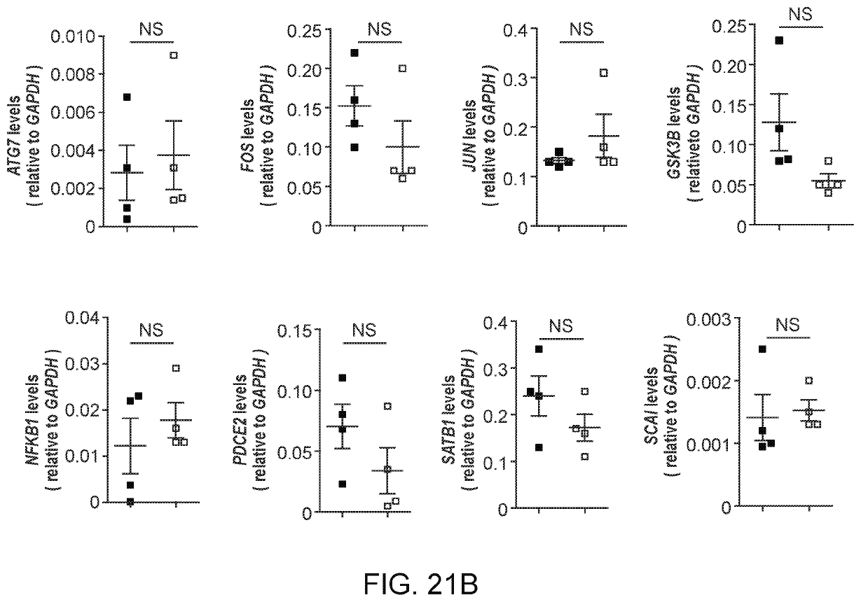

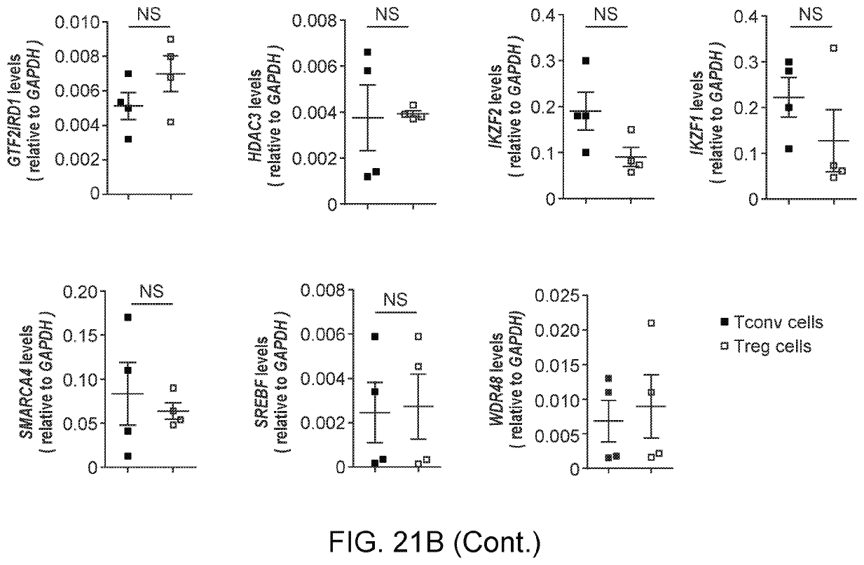

To understand the pathways involved in maintaining primary human Treg cells, first proteins interacting with FOXP3-regulating transcription factors in CD4+ T cells were examined. Proteomic data was used, which listed binding partners of NFAT (Gabriel et. al., 2016), STAT5 (Nanou et. al., 2017; Richard et. al., 2017), and SMAD3 (Grimsby et. al., 2004; Nold et. al., 2010). 20 proteins were identified based on their roles in transcriptional regulation and expression levels in human CD4+ T cells (FIG. 21A). Subsequently, the gene expression of these 20 proteins in purified human Treg and Tconv cells was analyzed and found that five (CHK1, CREB1, IL37, RPTOR, and RUNX1) were significantly upregulated in Treg cells compared to Tconv cells (FIGS. 21A, 37A-B).

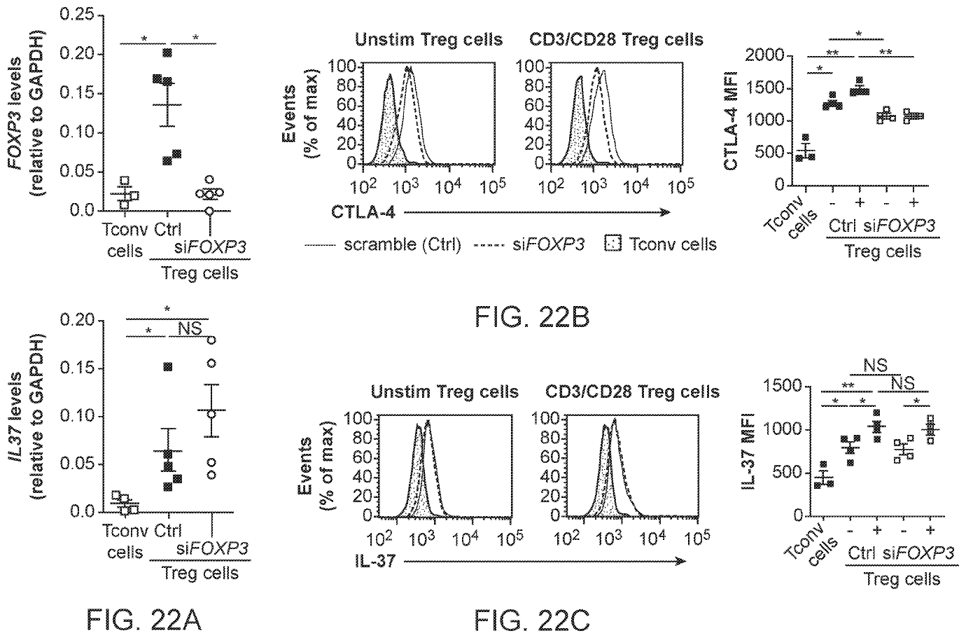

To investigate the necessity of CHK1, CREB1, IL37, RPTOR, and RUNX1 for FOXP3 expression in Treg cells, each gene in primary human CD4+ T cells was knocked down and measured their effects on FOXP3 protein expression. Although the knockdown of CHK1 and RPTOR had minimal impact on FOXP3 expression, the knockdown of CREB1, IL37, and RUNX1 resulted in a significant decline in the frequency of FOXP3+ T cells in CD4+ T cells (FIGS. 37C-D). Notably, IL37 knockdown exerted the most significant influence on FOXP3 expression in CD4+ T cells. IL-37 is a member of the IL-1 family with a unique anti-inflammatory and immunosuppressive role (Dinarello et. al., 2016). However, the direct role of IL-37 in T cells and its molecular mechanisms remain largely undefined. The regulatory effect of IL37 on FOXP3 transcription in Treg cells was further confirmed by an IL37 knockdown in Treg cells, which resulted in a 72% decrease in FOXP3 (FIG. 37E). Because FOXP3 is a master regulator of Treg cells, FOXP3 was knocked down in T cells to examine its effect on IL-37 expression. Whereas FOXP3 knockdown (FIG. 22A, top) resulted in a predicted reduction in CTLA-4 protein in both unstimulated and TCR/CD28-stimulated Treg cells (FIG. 3B), it did not affect IL37 mRNA levels (FIG. 22A, bottom) or intracellular protein amounts (FIG. 22C) in both unstimulated and TCR/CD28-stimulated Treg cells. These findings demonstrate that IL-37 expression enhances FOXP3 transcription, whereas FOXP3 does not regulate IL37 expression in human Treg cells, suggesting that IL-37 plays a critical role in Treg cell function.

### IL-37 Regulates FOXP3 at the Transcriptional Level in Human Treg Cells

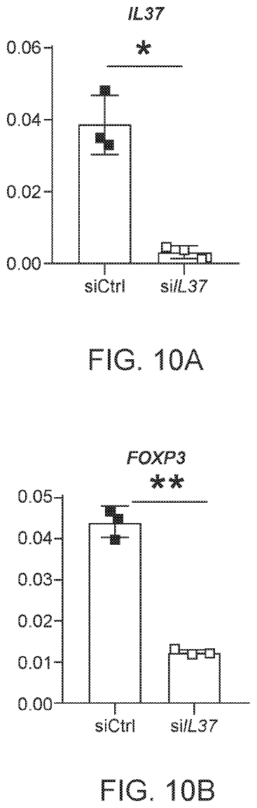

The effect of IL37 on the transcriptional regulation of FOXP3 was further confirmed. Gene expression of FOXP3 was examined following knockdown of IL37 expression in human Treg cells 24 hours after transfection with IL-37 siRNA (siIL37) compared to scramble siRNA control (siCtrl). Expression was analyzed by qRT-PCR. As a control, IL37 expression was also examined. As shown in FIG. 10A, transfection with siIL37 lead to a significant knockdown of IL-37 expression in Treg cells. FIG. 10B shows the knockdown of FOXP3 expression in Treg cells. As shown in FIG. 10B, knockdown of IL37 in Treg cells resulted in a 72% decrease of FOXP3 expression. These results demonstrate that the expression of IL-37 increases the transcription of FOXP3, suggesting a critical role for IL-37 in Treg cell function.

IL-37-Expressing Treg Cells are More Suppressive than IL-37-not-Expressing Treg Cells In Vitro and In Vivo

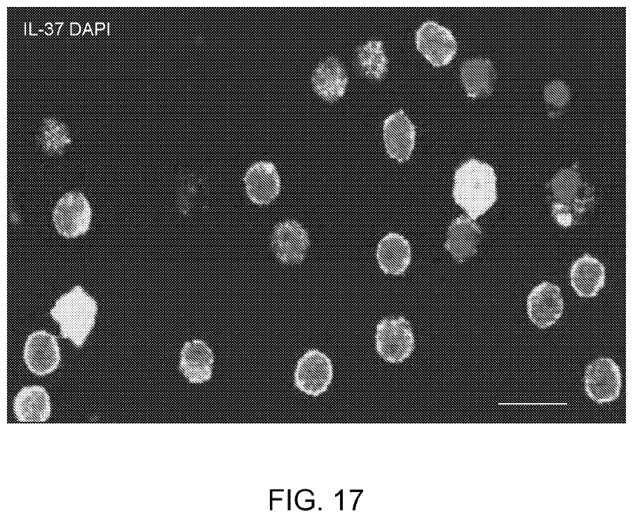

In measuring the contribution of IL-37 expression by immune cells present in human PBMCs using Primeflow® mRNA assay, a flow-based gene expression assay, it was discovered that the percent of IL37 mRNA expression is significantly higher in Treg cells than other T lymphocyte subsets, as shown in FIG. 1F. Detectable levels of IL-37 secretion were only found in the supernatant of Treg cells. IL-37 immunofluorescence staining of Treg cells confirmed IL-37 expression and that IL-37 was not ubiquitously expressed in all human Treg cells (FIG. 17).

Because FOXP3^{hi} Treg cells are more suppressive compared to other Treg cell subsets (Chauhan et. al., 2009; Miyara et. al., 2009; Strauss et. al., 2007; Wan and Flavell, 2007), it was speculated that IL-37^{hi} Treg cells possessed increased suppressive ability when compared to IL-37^{lo} Treg cells.

As previously reported, IL37 is highly expressed in human Treg cells (Osborne 2019, Osborne et. al., 2019, Osborne et. al., 2022). Flow cytometry-based assay to measure IL37 mRNA levels in freshly obtained human PBMCs without culture or fractionation was used. The PrimeFlow® assay was used to simultaneously measure the gene expression of a specific target, IL37, in multiple immune cell subsets and provide single-cell analyses. Cells were stimulated with LPS, similar to the condition from another study to induce IL37. The gating strategy in FIG. 1A (myeloid) and FIG. 1B (lymphoid cells) was performed to measure the quantity of IL37 in multiple immune cell types (FIG. 23A). As shown in FIG. 23B, the majority of monocytes (87%) and Treg cells (CD4+CD25^{hi}CD45RB^{lo}) (80%) expressed IL37 mRNA, and their frequencies increased further from 87% to 97% in monocytes and 80% to 91% in Treg cells after LPS stimulation. IL37 was expressed in more than 50% of other T cell populations (CD3+, CD8+, CD4+, and Tconv cells), and their frequencies increased after LPS stimulation. On the other hand, IL37 was expressed in only a portion of other populations (dendritic cells, B cells, and NK cells), and their frequencies were not changed after LPS stimulation.

IL37 expression levels in each cell type (FIG. 23C) was further analyzed. The IL37 mRNA level was highest in monocytes compared to other immune subsets, and the level further increased after LPS stimulation. Treg cells expressed the highest IL37 level among lymphoid subsets, which further increased after LPS stimulation. Based on these results and the results of others, it can be concluded IL37 is highly expressed in human Treg cells. Considering its role in inducing FOXP3, it is speculated that IL-37 would be a good candidate for establishing a Treg-like cell line from non-Treg CD4+ cells expressing lower IL-37.

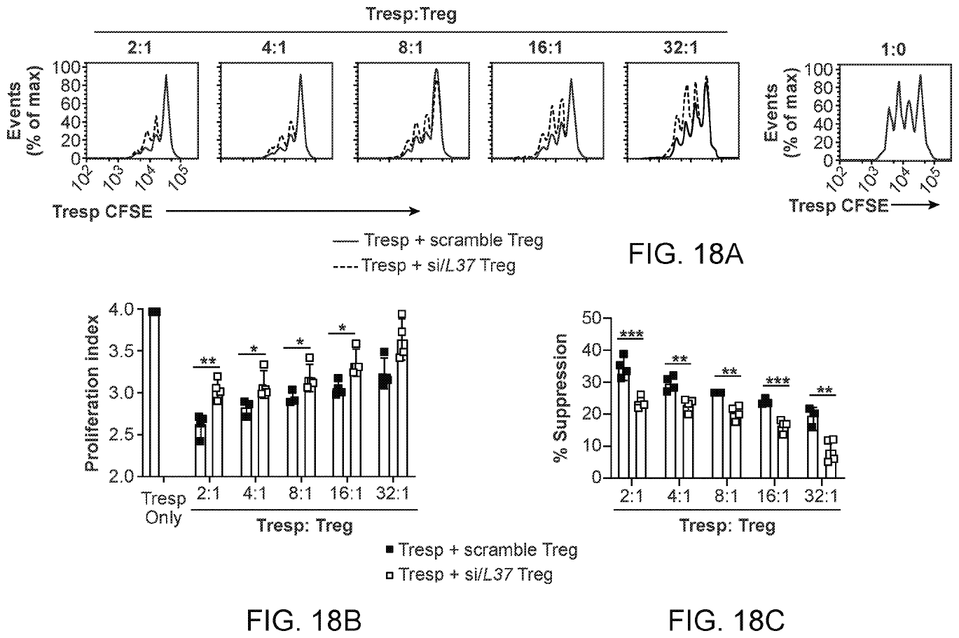

To investigate the role of IL-37 in human Treg suppressive function, IL-37 expression was knocked down in purified human Treg cells and the Treg cells were cultured at various dilutions with purified naïve CD4+CD25− T responder cells (Tresp) and an in vitro suppression assay was performed, as shown in FIGS. 18A-C. In vitro T cell suppression assay by human Treg cells transfected with scrambled siRNA (black) or siIL37 (gray). Histograms (FIG. 18A), proliferation indexes (FIG. 18B) and % suppression (FIG. 18C) show the division of CFSE-labeled CD4+CD25− T cell responders (Tresp), cultured with anti-CD3 and human Treg cells at Treg:Tresp ratios of 2:1 to 32:1 for 3 d. “Tresp only (Treg:Tresp ratio of 1:0)” in FIGS. 18A-B are Tᵣₑₛₚ cells cultured alone with anti-CD3 for 3 days and serve as a positive control. PI; proliferation index.

Compared to Treg cells transfected with scramble siRNA, siIL37-transfected Treg cells exhibited diminished suppressive abilities (FIG. 18A). FIGS. 18B-C show the significant reduction in the proliferation of Tresp cells cultured with Treg cells transfected with scramble siRNA compared to siIL37 Treg cells. Tresp cells cultured with siIL37 Treg cells saw an average increase in proliferation index (PI) at 10.6% (FIG. 18B) and an average decrease of 8.5% of suppression across all Tresp:Treg dilutions (FIG. 18C) suggesting that IL-37 bolsters the suppressive function of human Treg cells.

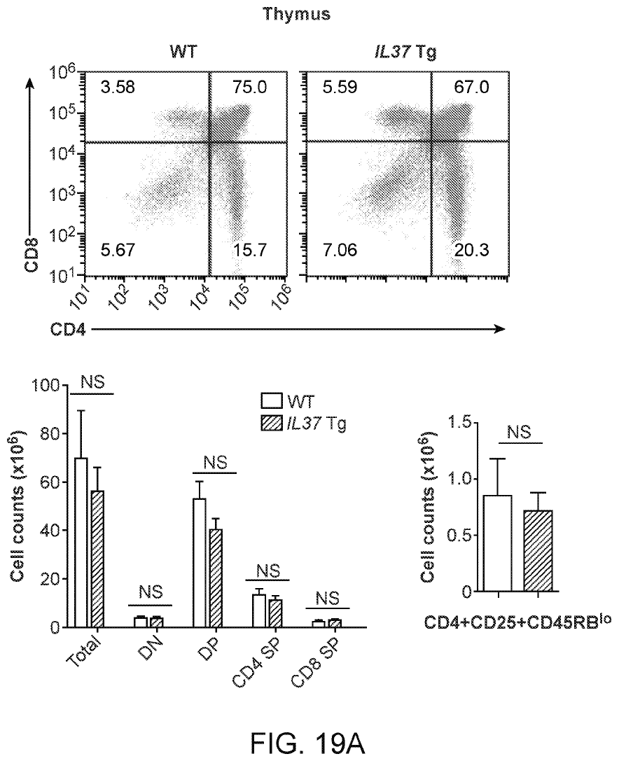

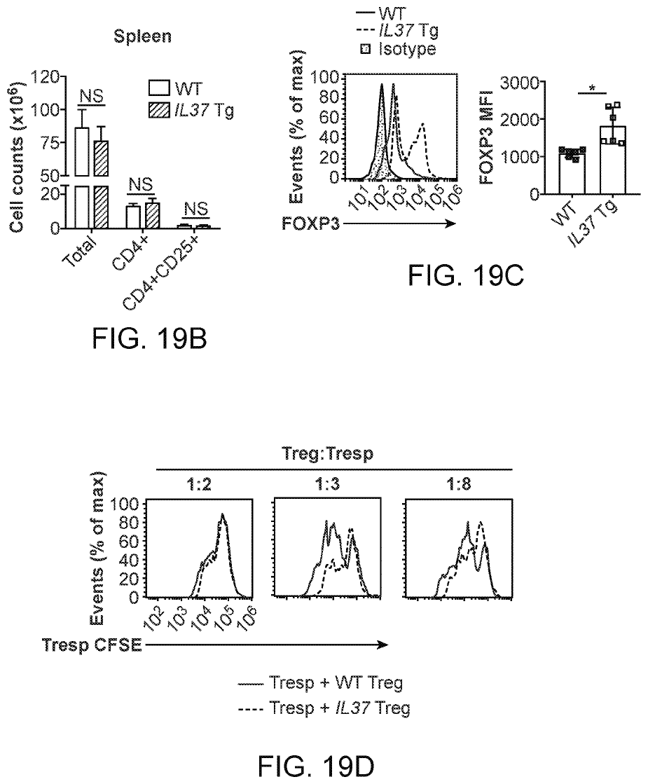

To further analyze the suppressive functions, purified Treg cells from wild-type (WT) and IL37 Tg mice were used. A mouse homolog for human IL37 has not been identified, and IL37 Tg mice express human IL37 under a CMV promoter (Nold et. al., 2010). To analyze the suppressive role of IL-37 in vivo, experiments were conducted using wild-type (WT) and IL37 Tg mice, which express human IL37 under a CMV promoter (Nold et. al., 2010). Although a mouse homolog for human IL37 has not been identified, human IL-37 proves functional in mouse cells, and as such, IL37 Tg mice have been widely utilized to study IL-37 functionality by many research groups (Dinarello et. al., 2016). Prior to investigating Treg cells from IL37 Tg mice, T cell development was examined and confirmed that the absolute numbers of thymocytes and T cells in the thymus and spleen were consistent between WT and IL37 Tg mice (FIGS. 19A-B). However, Treg cells isolated from the spleens of IL37 Tg mice (IL37 Treg cells) displayed a 1.67-fold increase in Foxp3 protein expression compared to those from WT mice (WT Treg cells) (FIG. 19C). These findings align with our earlier results showing that IL-37 enhances FOXP3 expression in human cells (FIGS. 37C-E).

Whereas both WT and IL37 Treg cells were functionally suppressive in vitro, IL37 Treg cells more effectively suppressed the proliferation of naïve CD4+CD25− T responder (Tresp) cells isolated from WT mice compared to WT Treg cells (FIG. 19D). FIGS. 7B-C show that both WT and IL37 Treg cells were functionally suppressive in vitro and inhibited the proliferation of naïve CD4+CD25− T responder (Tresp) cells isolated from WT mice; however, Tresp cell proliferation was suppressed more by IL37 Treg cells compared to WT Treg cells, as shown in FIG. 7C. The suppressive activity of Treg cells was further analyzed in vivo using a contact-hypersensitivity (CHS) model where hapten (dinitrofluorobenzene, DNFB)-sensitized WT mice were adoptively transferred with WT or IL37 Treg cells and subsequently challenged with DNFB. A schematic of the CHS model is shown in FIG. 8A. Whereas the CHS responses in sensitized WT mice after DNFB challenge were ameliorated by the adoptive transfer of IL37 Treg cells (52% and 58% reduction at 24 and 48 h, respectively), as shown in FIG. 8B, such reduction was not observed in mice receiving WT Treg cells (FIG. 8B). The histology showed a significant reduction in epidermal hyperplasia, dermal edema, vasodilation, and dermal inflammatory infiltrates in the ears of mice receiving IL37 Treg cells but not in those receiving WT Treg cells, as shown in FIG. 8C. These data demonstrate that IL37 Treg cells are highly suppressive in vitro and in vivo compared to WT Treg cells.

The lack of CHS suppression by WT Treg cells despite their in vitro suppressive ability suggested that adoptively transferred WT Treg cells either died, failed to migrate to the inflamed tissues, or lost their suppressive function in vivo. To explore these possibilities, adoptively transferred Treg cells were tracked (Treg gating strategy in FIG. S4A) using CFSE. CFSE-labeled T cells were detected in the DNFB-challenged ear tissue specimens (FIG. 8D) from both mice that received WT Treg cells and those that received IL37 Treg cells, confirming the successful migration and survival of the adoptively transferred cells in the inflamed skin. Interestingly, it was noted a higher number of CFSE+ T cells in mice that received WT Treg cells than those that received IL37 Treg cells. These results suggested that, despite their increased number, adoptively transferred WT Treg cells might have lost their suppressive ability within the inflamed skin, thereby failing to suppress CHS.

Tracking of CFSE-labeled adoptively transferred Treg cells revealed their presence in the DNFB-challenged ear tissue specimens in both mice that received either WT Treg cells or IL37 Treg cells, as shown in FIGS. 9B-C, confirming the migration and survival of adoptively transferred Treg cells into the inflamed skin. The increased number of CFSE+ T cells in mice that received WT Treg cells compared to IL37 Treg cells suggested that, despite their increase, adoptively transferred WT Treg cells have lost their suppressive ability in the inflamed skin.

Elevated expression of IL-37 in Treg cells stabilizes FOXP3 expression and prevents Treg cell conversion to Tconv cells in inflammatory conditions

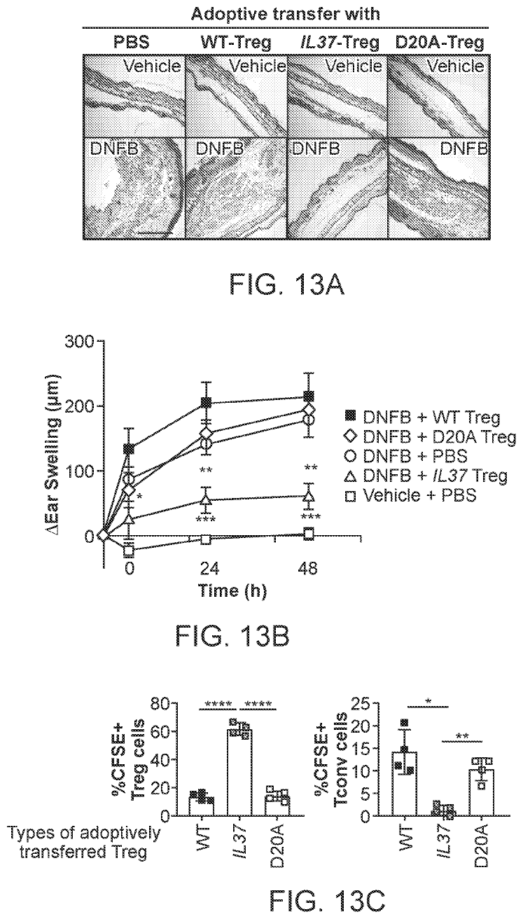

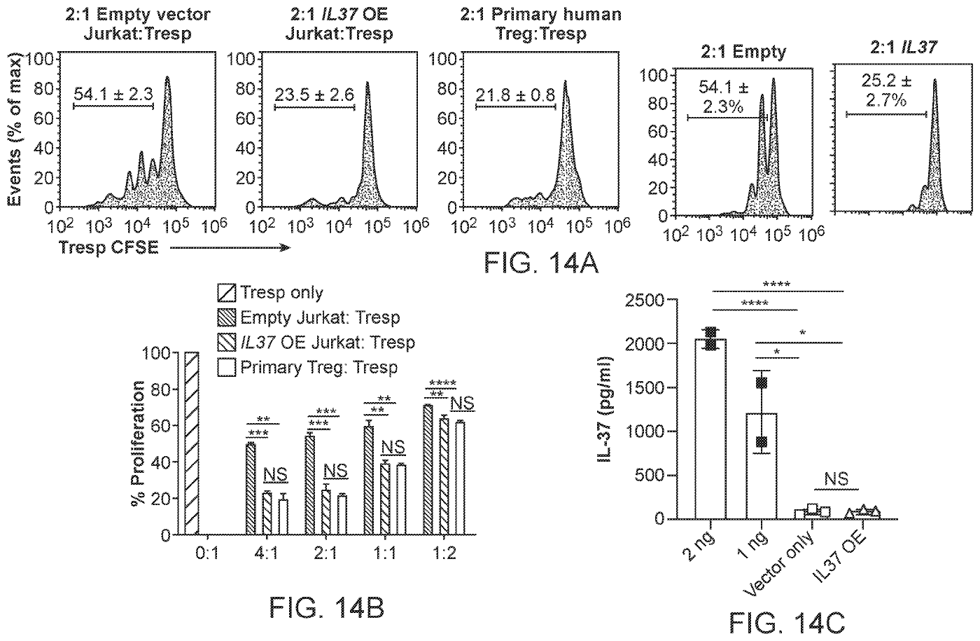

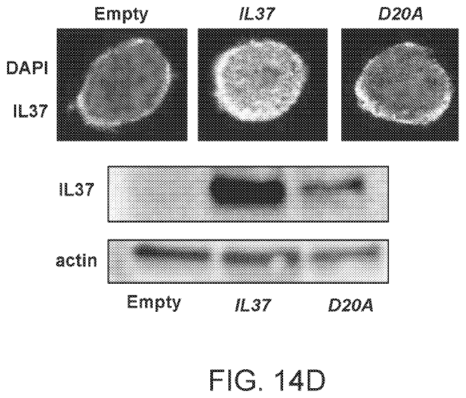

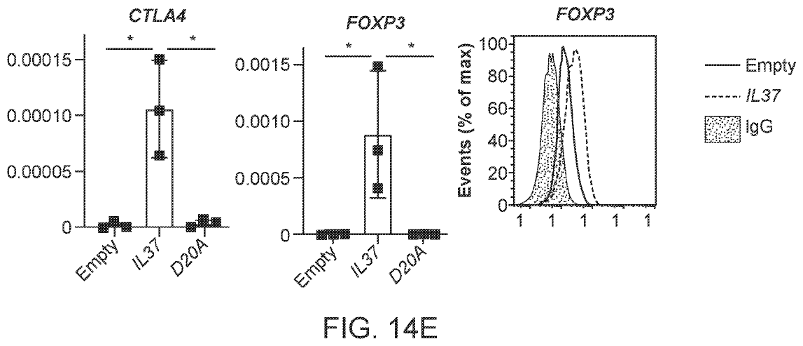

Under inflammatory or pathogenic circumstances, some Treg cells lose FOXP3 expression and their suppressive function (Gao et. al., 2012; Komatsu et. al., 2014; Zhou et. al., 2009). Therefore, it was speculated that CFSE-labeled adoptively transferred WT Treg cells could have lost Foxp3 expression, leading to a failure in maintaining their suppressive ability in vivo. Phenotypic analysis of the CHS skin in mice that received WT Treg cells revealed an appearance of CFSE+ Tconv cells and a reduction in CFSE+ Treg cell frequency, suggesting the reprogramming of some WT Treg cells to Tconv cells in vivo, as shown in FIGS. 13A-C.

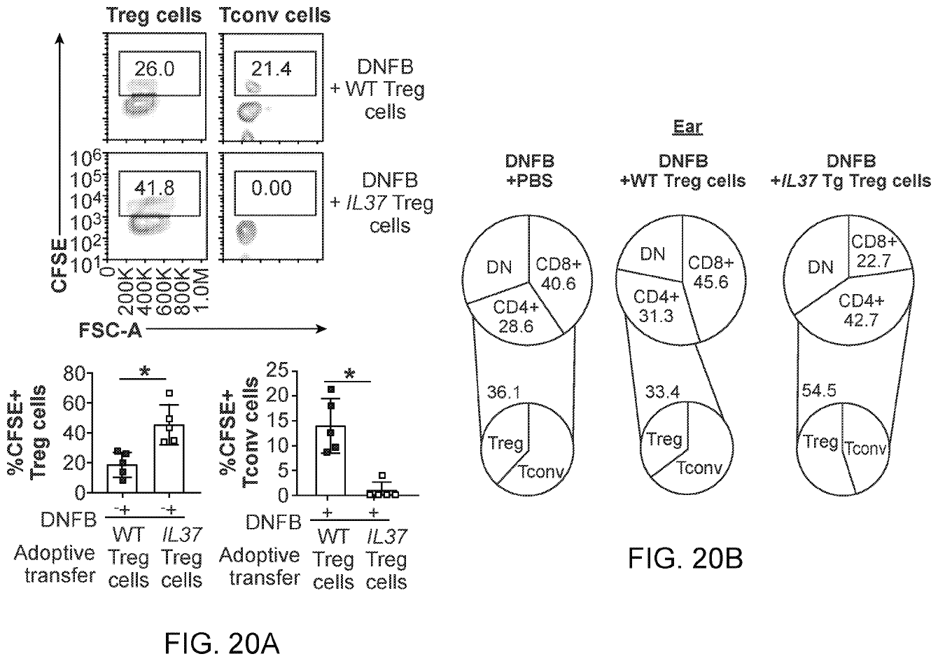

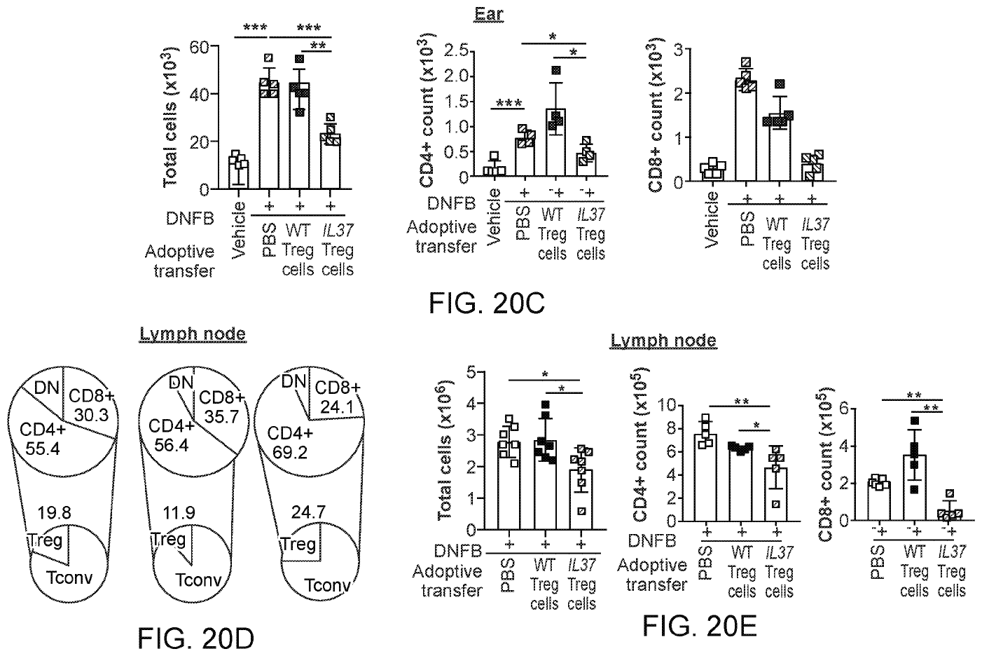

Detailed analysis of the CHS skin in mice in FIG. 8 revealed the emergence of CFSE+ CD4+CD25^{hi}CD45RB^{lo} (Tconv) cells and a decrease in the frequency of CFSE+ CD4+CD25^{hi}CD45RB^{lo} (Treg) cells in mice that received WT Treg cells (FIG. 20A), suggesting that some WT Treg cells might have been reprogrammed into Tconv cells in vivo. Concurrently, these Treg cells failed to decrease infiltrating cell numbers, as well as CD4+ and CD8+ T-cell counts in the inflamed skin (FIGS. 8B-C) and draining lymph nodes (dLN) (FIGS. 20D-E). Conversely, the inflamed skin of mice that received IL37 Treg cells did not contain Tconv cells (FIG. 20A). As a result, Treg cell frequency increased (FIGS. 20B,D), coupled with a reduction in infiltrating cell numbers, as well as CD4+ and CD8+ T-cell counts in the inflamed skin (FIGS. 20B-C) and dLN (FIGS. 20D-E). These findings suggest that IL-37 is crucial in maintaining Foxp3 expression in Treg cells in inflammatory conditions.

Because IL-37 upregulates FOXP3 in Treg cells, it was examined whether high expression of IL-37 in Treg cells resulted in maintaining FOXP3 expression and preventing Treg cell instability in inflammatory conditions. To investigate whether the expression of IL-37 in Treg cells leads to sustained Foxp3 expression and prevents Treg cell instability in inflammatory conditions, IL-6 was added in Treg cells. IL-6 is known to downregulate Foxp3 expression by activating Stat3 and counteracting Stat5 signaling within Treg cells (Yao et. al. Blood 109:4368, 2007). The introduction of IL-6 to the WT Treg cells significantly reduced both mRNA (94% decrease, FIG. 9A) and protein (20% decrease, FIG. 9B) levels of Foxp3, which aligns with previous studies (Bettelli et. al., 2006; Y. Gao et. al., 2015; Z. Gao et. al., 2012; Zheng et. al., 2008). However, in IL37 Treg cells, the reduction of Foxp3 was not observed in the presence of IL-6. Of note, IL-37 levels remained not decreased by the addition of IL-6 in IL37 Treg cells (FIG. 9C). These results were further corroborated by testing human Treg cells. When human Treg cells were stimulated with anti-TCR/CD28, the introduction of IL-6 led to a 34% decrease in the frequency of CD25+FOXP3+ cells within the gated IL-37^{lo} human Treg cell population (FIG. 9D). However, no such reduction was observed in the gated IL37^{hi} human Treg population. These findings demonstrate that IL-37 expression within Treg cells not only regulates FOXP3 expression at the transcriptional level but also stabilizes FOXP3 expression under inflammatory conditions in vitro.

The observed reduction in FOXP3 expression and suppressive function in WT Treg cells under inflammatory conditions, as well as IL-37^{lo} or IL37 knockdown human Treg cells, suggest that Treg cells without IL-37 expression may be converted to Tconv cells and lose their immunosuppressive characteristics under inflammatory conditions. To determine if IL37 knockdown prompts Treg cells into another T lymphocyte subset, alterations in T cell differentiation-specific gene expression were evaluated (FIG. 9E). Changes were noted in the common Th lineage transcription factors (TBET, GATA, and RORγT) (Zhu et. al., 2010) within siIL37 Treg cells, with an appropriate 1.3-fold increase in TBET, a 1.5-fold increase in RORγT, and a roughly 5.5-fold decrease in GATA. The elevated TBET, RORγT, and lower FOXP3 gene expression after IL37 knockdown indicate that the gene expression pattern in siIL37 Treg cells is similar to a Th17 lineage (Nalbant and Eskier, 2016). To verify if siIL37 Treg cells transition into a Th17 lineage, Th17-specific genes was measured and found increased expression of STAT3 (˜2.9-fold), IRF4 (˜5.7-fold), and IL17 (˜1.24-fold) in siIL37 Treg cells compared to control Treg cells. These findings align with those from WT and IL37 Tg Treg cells under inflammatory conditions (FIG. 9F). Similar to human siIL37 Treg cells treated with IL-6, WT Treg cells showed a 4-fold average increase in Th lineage markers Gata3 and Rorγt, along with increased expression of Th17 markers Irf4 and Il17. On the other hand, no such increase was observed in IL-6-treated IL37 Tg Treg cells, supporting the idea that IL-37 expression in Treg cells prevents their conversion into other T cell lineages and enhances Treg cell stability. These results demonstrate that IL-37 expression in Treg cells not only controls FOXP3 expression but also prevents the reduction of FOXP3 expression at both the mRNA and protein levels in inflammatory conditions in vitro, leading to the stability of FOXP3 expression.

IL-37 Interacts with p-SMAD3 S208 and Translocates into the Nucleus to Induce FOXP3 Expression in Human Treg Cells

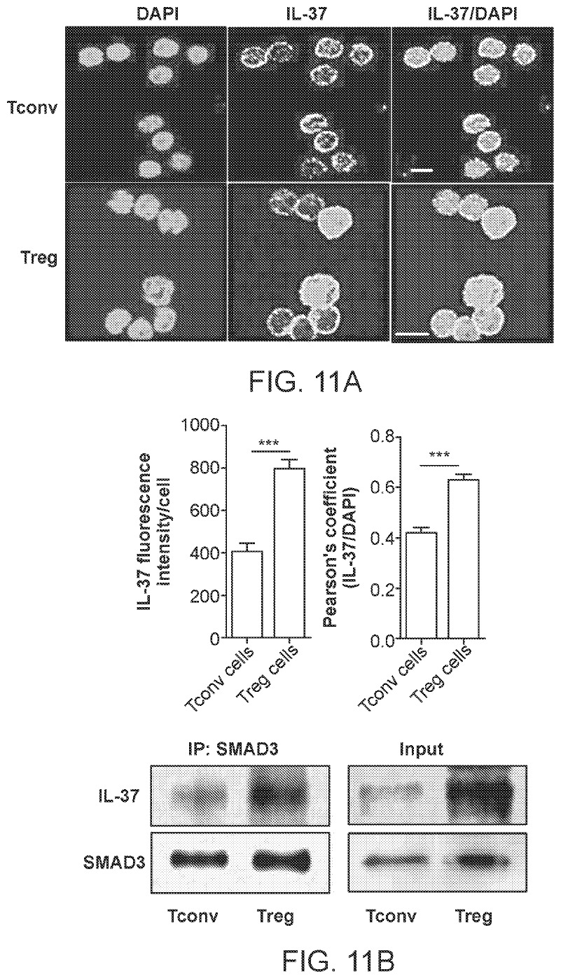

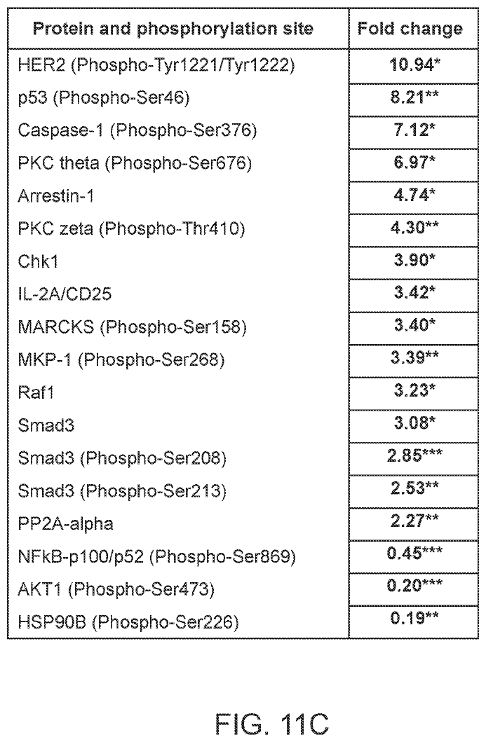

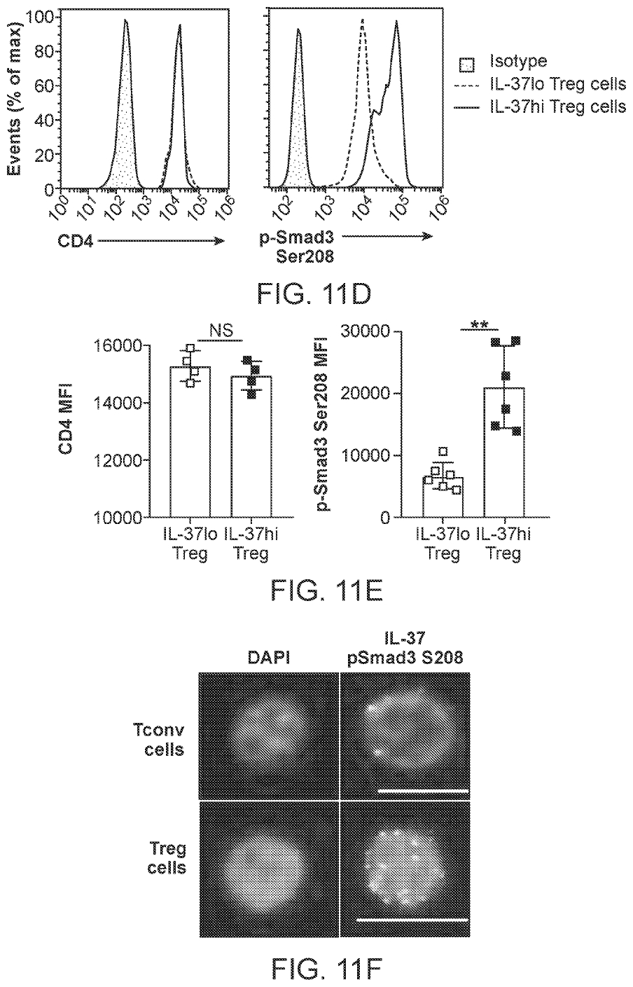

Next, the mechanism of IL-37-driven FOXP3 transcription in human Treg cells was investigated. In macrophages, IL-37 is present in the cytoplasm but upon LPS stimulation it translocates to the nucleus (Bulau et. al., 2014). IL-37 has a weak nuclear localization site and binds to transcription factors, particularly Smad3 (Grimsby et. al., 2004; Nold et. al., 2010), an important transcription factor for FOXP3 expression. IL-37 does not contain a DNA-binding domain. However, IL-37 has a weak nuclear localization site and was reported to bind to SMAD3 in macrophages. It is speculated that SMAD3 transported IL-37 to the nucleus, leading to the transcriptional regulation of FOXP3. (Bulau et. al., 2014). Given that IL-37 has a weak nuclear localization site and can bind to transcription factors, notably SMAD3 (Grimsby et. al., 2004; Nold et. al., 2010), which plays a crucial role in FOXP3 expression, the subcellular localization of IL-37 was investigated and its interaction with SMAD3 in human Treg cells. Through immunofluorescence analysis, it was observed that IL-37 exhibited a higher expression level (as indicated by fluorescence intensity) and prominent nuclear localization (as evidenced by colocalization with the nuclear stain DAPI) in human Treg cells compared to Tconv cells (FIG. 11A). Immunoprecipitation analyses further revealed an interaction of IL-37 with SMAD3 in human Treg cells (FIG. 11B), suggesting a potential role for IL-37 and SMAD3 in the transcriptional regulation of FOXP3 human Treg cells.

To analyze the protein changes associated with IL-37 expression in Treg cells, a phospho-proteomic analysis of mouse WT and IL37 Tg Treg cells was conducted (FIG. 11C). Three downregulated phosphoproteins in IL37 Treg cells compared to WT Treg cells (greater than 2-fold change and p-value <0.05) were investigated, including p-AKT1 (S473). Notably, p-AKT1 (S473) is a crucial protein that is activated in T effector cells and inhibited in Treg cells following TCR signaling (Crellin et. al., 2007; Li and Rudensky, 2016; Zanin-Zhorov et. al., 2010). Furthermore, six proteins (Arrestin-1, CHK1, IL-2RA/CD25, RAF1, Smad3, and PP2A-alpha) were found to be upregulated, along with nine upregulated phosphoproteins, including p-HER2 (Y1221/Y1222), p-P53 (S46), p-caspase-1 (S376), p-PKC theta (S676), p-PKC zeta (T410), p-MARCKS (S158), p-MKP-1 (S268) and p-Smad3 (S208 and S213) in IL37 Tg Treg cells. Using human Treg cells, elevated protein levels of CD25 were confirmed (not shown), one of FOXP3 targets, SMAD3 (Basak et. al., 2005), and p-SMAD3 (S208), in IL-37^{hi} human Treg cells compared to IL-37^{lo} human Treg cells (FIGS. 11D-E, CD4 used as a control). Given the observed elevation in SMAD3 and p-SMAD3 S208 compared to other SMAD3 phosphorylation sites in the phospho-proteomic analyses, the interaction between IL-37 and p-SMAD3 S208 was examined. An immunofluorescence-based proximity ligation assay (PLA) revealed that IL-37 binds to p-SMAD3 S208 in both the nucleus and cytoplasm of human Treg cells (FIG. 11F). These findings suggest that the interaction of IL-37 with p-SMAD3 S208 and its subsequent translocation into the nucleus contributes to the transcriptional regulation of FOXP 3 and the highly suppressive phenotype observed in human Treg cells.

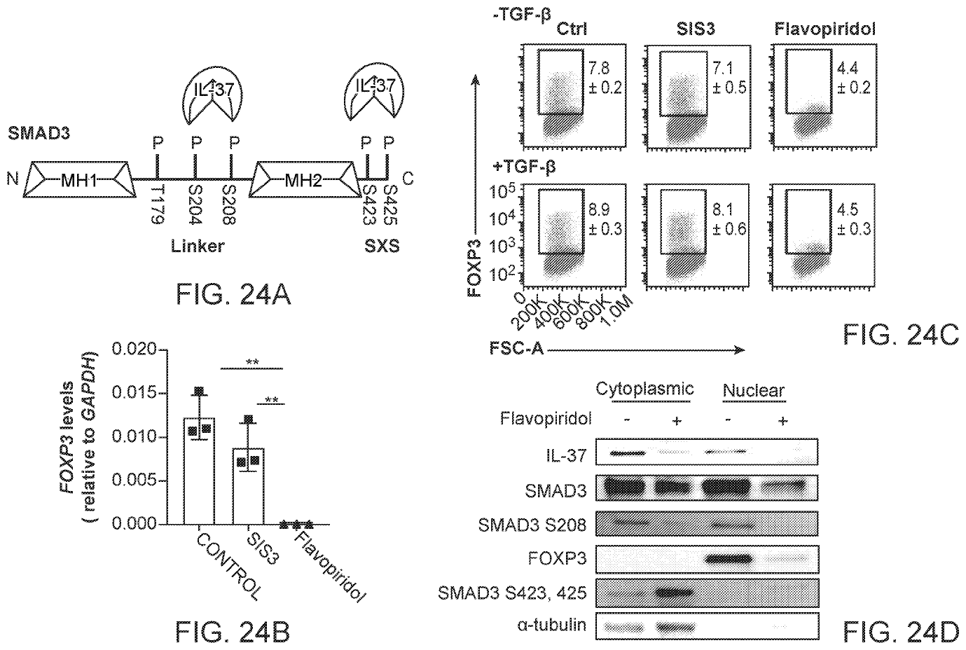

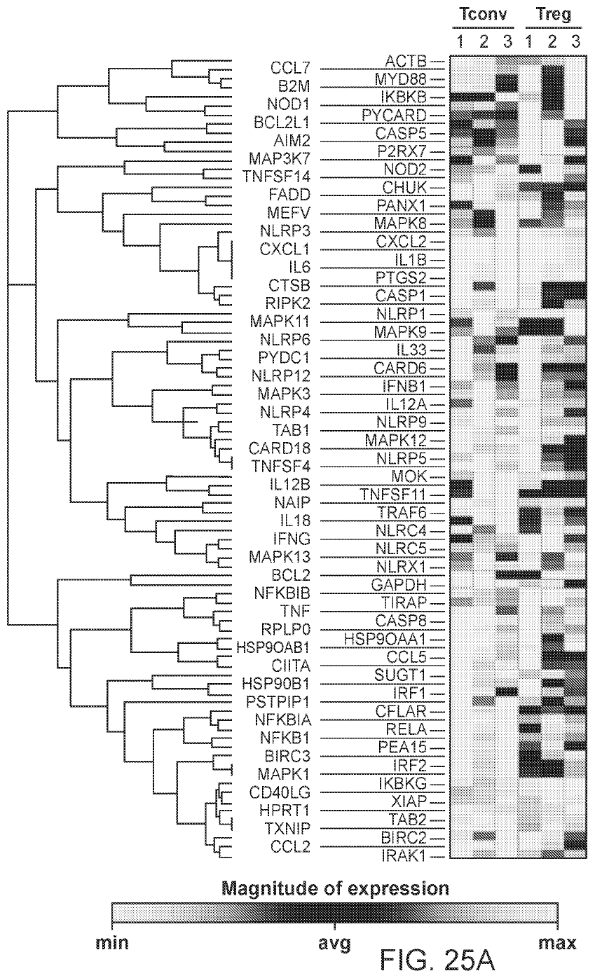

p-SMAD3 S423/425 is recognized for its involvement in TGFβ signaling. While treatment of CD4+ T with the p-SMAD3 S423/S425 inhibitor SIS3 has been shown to decrease Foxp3 expression in mouse Treg cells and EL4 cells (Tone et. al., 2008), our phospho-proteomic analysis showed no significant changes in p-Smad3 S423/425 levels between mouse WT and IL37 Tg Treg cells. Given the essential role of SMAD3 phosphorylation in nuclear localization and transcriptional activity controlling other genes, the phosphorylation status and its association with IL-37 in human Treg cells was examined (FIG. 24A). Inhibitors of S423/425 sites (SIS3) and T179/S208 sites (flavopiridol) were utilized to determine if they could decrease FOXP3 mRNA expression and disrupt the SMAD3/IL-37 interaction. Treatment of human Treg cells with SIS3 did not significantly alter FOXP3 mRNA expression. However, flavopiridol treatment almost completely obliterated FOXP3 expression in human Treg cells (FIG. 24B). This result was corroborated at the protein expression level. It was found that FOXP3 expression in a population of primary human CD4+ T cells resulted in a ˜2-fold decrease compared to control cells and was not rescued by the addition of TGF-β (FIG. 24C). Conversely, SIS3 treatment did not significantly decrease FOXP3 expression, and TGF-β even slightly increased FOXP3 expression. These findings suggest that the phosphorylation of SMAD3 at T179/S208 sites but not at S423/425 sites plays a vital role in regulating FOXP3 mRNA, even in the presence of TGF-β signaling. These findings also point to a crucial interplay between p-SMAD3 S208 and IL-37 in orchestrating the transcriptional regulation of FOXP3 in human Treg cells.

Therefore, cytoplasmic and nuclear proteins of primary human Treg cells was scrutinized following flavopiridol treatment to ascertain if p-SMAD3 S208 is essential for the nuclear localization of SMAD3 and IL-37. Immunoblotting for cytoplasmic and nuclear p-SMAD3 S208 along with total SMAD3 in FIG. 24D revealed that flavopiridol not only diminished p-SMAD3 S208 but also lowered nuclear SMAD3 levels, indicating that S208 is essential for SMAD3's nuclear entry. Concurrently, flavopiridol reduced the nuclear levels of FOXP3 and abolished the expression of IL-37 in the nucleus, suggesting that IL-37 requires p-SMAD3 S208 for nuclear entry and subsequent induction of FOXP3 expression. Interestingly, p-SMAD3 S423/S425 was undetectable in the nucleus until post-flavopiridol treatment, implying that in the absence of p-SMAD3 S208, p-SMAD3 S423/S425 might serve as an alternative route to induce FOXP3 expression in Treg cells. These data bolster the hypothesis that p-SMAD3 S208 is crucial for the interplay between SMAD3 and IL-37, enabling their nuclear entry and subsequent FOXP3 expression induction.

### Caspase-1 Activity is Required for Nuclear Localization of IL-37 and its Function to Maintain FOXP3 Expression and Immune Suppression in Inflammatory Conditions

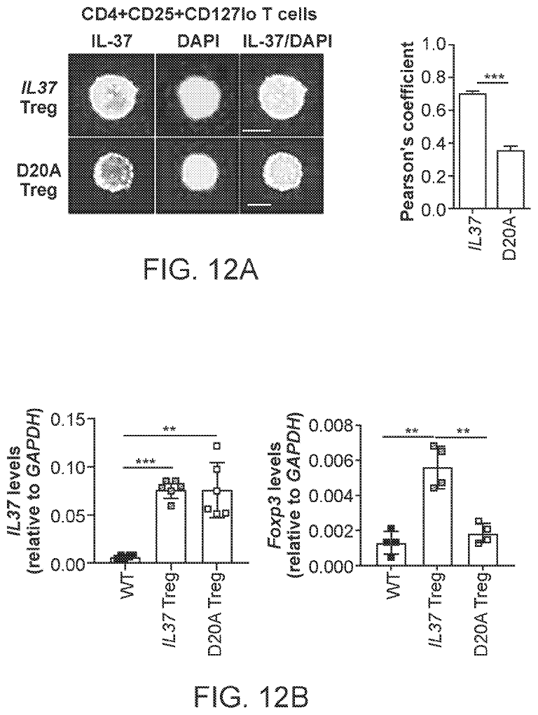

The translocation of IL-37 is caspase-1 dependent in macrophages (Bulau et. al., 2014). To analyze the requirement of caspase-1 activation and subsequent cleavage of transgenic IL-37 for its translocation to the nucleus and induction of Foxp3 transcription, IL37^{D20A} Tg (D20A) mice carrying a mutation in the caspase-1 cleavage site, which prevents IL-37 cleavage, were used. Unstimulated IL-37Tg and D20A Treg cells were stained with IL-37 (AF488) and DAPI. As shown in FIG. 12A, immunofluorescence imaging analysis showed a significant decrease in IL-37 localization to the nucleus in D20A Treg cells. IL-37 and FOXP3 expression was analyzed by qRT-PCR in WT, IL-37Tg and D20A Treg cells. GAPDH served as an internal control. Although this localization change was not associated with IL37 expression changes in D20A Treg cells, D20A Treg cells expressed significantly lower Foxp3 mRNA than IL37 Treg cells, as shown in FIG. 12B. Indeed, its expression was similar to that of WT Treg cells, demonstrating that IL-37 cleavage by caspase-1 was required for its nuclear translocation and Foxp3 induction in Treg cells.

### Cleavage by Caspase-1 is Required for Immune Suppression in Mice

The suppressive function of D20A Treg cells in vivo was investigated by adoptively transferring WT, IL37 Tg-Treg, and D20A Treg cells into DNFB-sensitized WT mice, which were subsequently challenged with DNFB, using the same CHS model workflow depicted in FIG. 8A. CHS responses were measured by ear thickness at 0, 6, 24 and 48 hours after vehicle or DNFB challenge in WT mice adoptively transferred with PBS, WT Treg cells, IL37 Tg Treg cells or D20A Treg cells. H&E staining was performed on ear specimens taken at 48 hours after DNFB challenge from mice adoptively transferred with PBS, WT-Treg, IL37 Tg or D20A Treg cells. Consistent with previous findings, the adoptive transfer of IL37 Treg cells suppressed CHS responses after the challenge, whereas this suppression was not observed in mice receiving WT or D20A Treg cells, as shown in FIGS. 13A-B. In particular, as shown in FIG. 13A, the reductions in epidermal hyperplasia, dermal edema, and dermal inflammatory infiltrates observed in mice receiving IL37 Treg cells were not observed in mice receiving WT or D20A Treg cells.

FIG. 13C shows the frequency of CFSE-labeled cells among Treg cells (CD4+CD25hiCD45RBlo) (left) and Tconv cells (CD4+CD25loCD45RBhi) (right) from ear specimens, taken at 48 h after DNFB challenge from mice adoptively transferred with WT-Treg, IL-37-Treg or D20A-Treg cells. Similar to the effect seen in mice that received WT Treg cells, inflamed skin tissues demonstrated a decrease in CFSE+ Treg cell frequency and the presence of CFSE+ Tconv cells in mice that received D20A Treg cells, as shown in FIG. 13C. These data demonstrate that IL37 requires caspase-1 to localize to the nucleus to maintain Treg phenotypes and be highly suppressive in vivo under inflammatory conditions.

In summary, these results identify a potentially crucial role for the dual-function cytokine, IL-37, in controlling FOXP3 expression, Treg stability, and Treg function. The expression of IL-37 in human Treg cells may upregulate and stabilize FOXP3, thereby maintaining Treg stability and highly suppressive function. This work may uncover a new mechanism controlling the stability and plasticity of human Treg cells, which will have an immense translational impact on immune tolerance, autoimmunity, and transplantation.

### Caspase-1 Activity is Required for Nuclear Localization of IL-37 and its Function to Maintain FOXP3 Expression and Immune Suppression in Inflammatory Conditions

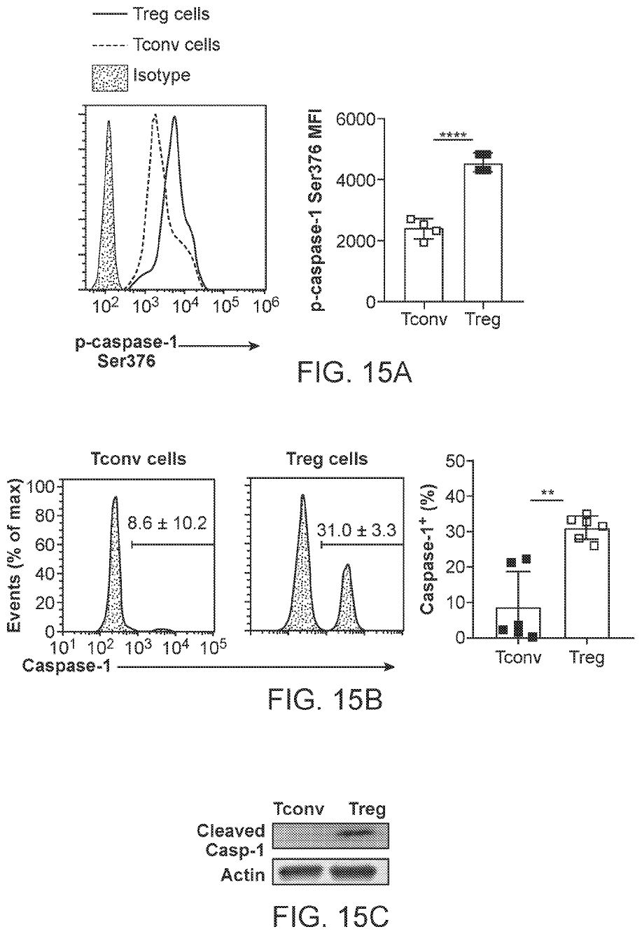

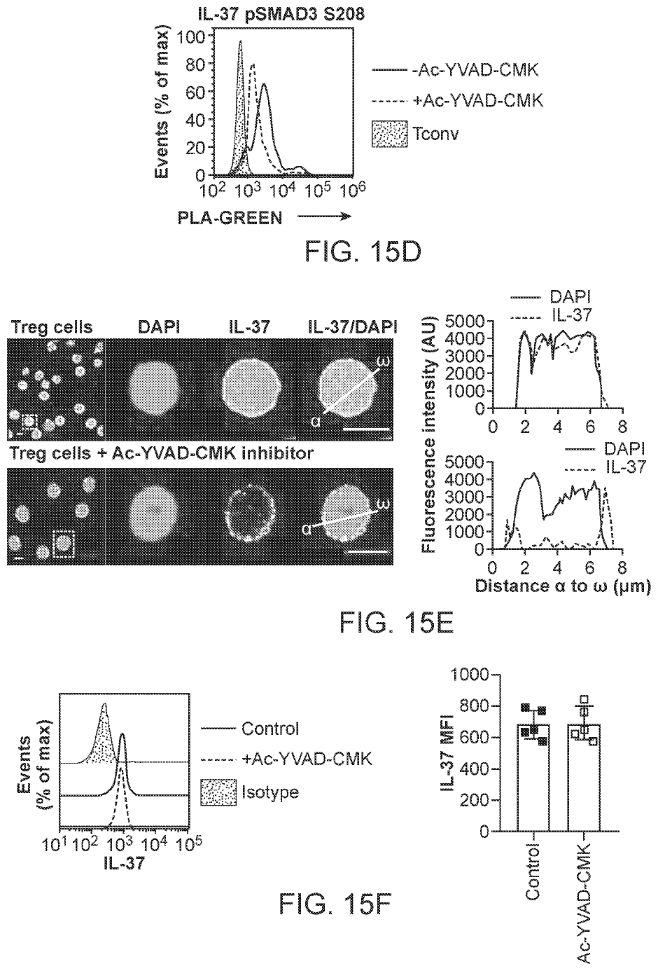

Therefore, the expression of caspase-1 and the effects of caspase-1 inhibition on IL-37 localization and FOXP3 expression in human Treg cells was examined. In macrophages, the translocation of IL-37 from the cytoplasm to the nucleus relies on caspase-1 (Bulau et. al., 2014). Our phospho-proteomic analysis (FIG. 11C) indicated elevated levels of p-caspase-1 S376, a phosphorylated form of caspase-1 involved in its activation (Basak et. al., 2005), in IL37 Tg Treg cells compared to WT Treg cells. Therefore, caspase-1 activity was explored and the impact of its inhibition on IL-37 localization and FOXP3 expression in human Treg cells. Unstimulated human Treg cells displayed higher phospho-caspase-1 (at S376) expression (FIG. 15A) and elevated caspase-1 enzymatic activity compared to Tconv cells (FIG. 15B). Treating human Treg cells with the caspase-1 inhibitor (Ac-YVAD-CMK) led to a diminished interaction between IL-37 and p-SMAD3 S208 (36.5% decrease) (FIG. 15D) and the shift of IL-37 localization from the nucleus to the perinuclear region of the cytoplasm (FIG. 15E) without alteration in IL-37 expression (FIG. 15F). These results indicate that IL-37 nuclear entry necessitataes active caspase-1.

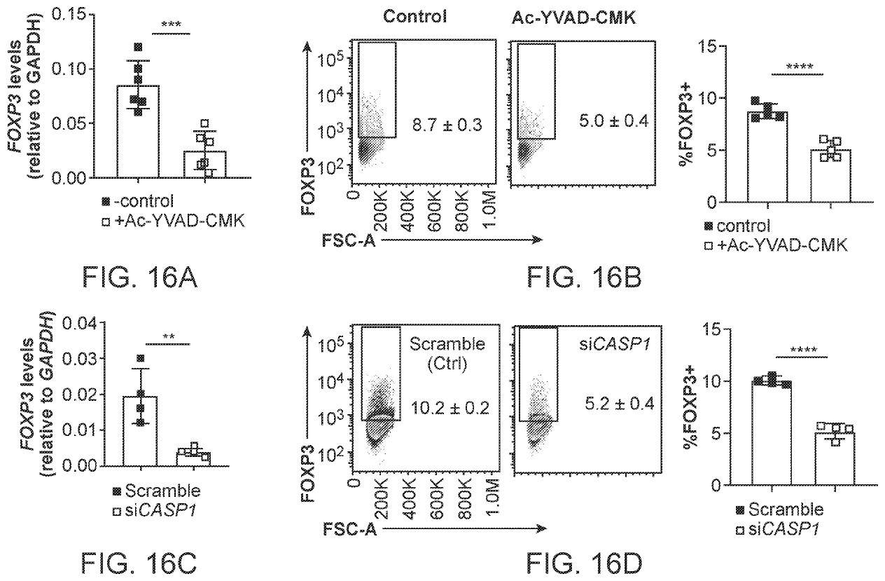

Next, it was investigated to determine if caspase-1 was indispensable for IL-37's regulation of FOXP3 expression. The use of the caspase-1 inhibitor Ac-YVAD-CMK during the culture of human CD4+ T cells resulted in the reduction of FOXP3 mRNA (FIG. 16A) and protein (FIG. 12G) expression. These results were corroborated with siRNA against caspase-1, CASPI, in a human CD4+ T cell population (FIGS. 16C-D). As shown in FIG. 15E, upon treatment with the caspase-1 inhibitor (Ac-YVAD-CMK), the subcellular localization of IL-37 in human Treg cells changed from the nucleus to the perinuclear region of cytoplasm.

It was examined whether caspase-1 was required for IL-37's regulation of FOXP3 expression. Culturing human T cells with the caspase-1 inhibitor Ac-YVAD-CMK resulted in the reduction of FOXP3 mRNA, as shown in FIG. 16B, and protein (FIG. 16B) expression without affecting IL-37 expression. These results were confirmed using siRNA against caspase-1 in a population of human CD4+ T cells (FIGS. 12H-I). FIG. 12H shows qRT-PCR analysis of FOXP3 gene expression following siRNA knockdown of caspase-1 in a population of human CD4+ T cells. GAPDH served as internal control. FIG. 12I shows contour plots (left) of FOXP3+ cell gating in human CD4+ T cells transfected with scramble siRNA (Scramble; Ctrl) and siCasp-1 48 h prior, as well as quantification of the percentage of FOXP3+ T cells (right). To further analyze the requirement of caspase-1 activation and subsequent cleavage of IL-37 for its translocation to the nucleus and induction of FOXP3 transcription, IL37^{D20A} Tg (D20A) mice carrying a mutation in the caspase-1 cleavage site that prevents IL-37 cleavage and thus nuclear translocation (Li et. al., 2019) were used.

To further analyze the requirement of caspase-1 activation and subsequent cleavage of IL-37 for its nuclear translocation and induction of FOXP3 transcription, IL37^{D20A} Tg (D20A) mice carrying a mutation in the caspase-1 cleavage site that prevents IL-37 cleavage and thus nuclear translocation were utlized (Li et. al., 2019). Confocal image analyses revealed a significant decrease in nuclear IL-37 localization in D20A Treg cells (FIG. 12A). While this alteration was not associated with changes in IL37 expression in D20A Treg cells (FIG. 12B), D20A Treg cells displayed significantly lower Foxp3 mRNA than IL37 Treg cells. Indeed, the Foxp3 expression in D20A Treg cells was similar to that of WT Treg cells, underscoring the necessity of IL-37 cleavage by caspase-1 for the nuclear translocation of IL-37 and the subsequent induction of Foxp3 expression in Treg cells.

### NLRC4 Inflammasome and Upstream Signaling are Required for IL-37-Directed FOXP3 Expression in Human Treg Cells

Caspase-1 activation requires forming and activating a proinflammatory innate immune multiprotein complex known as the inflammasome (Broz and Dixit, 2016). The detection of elevated active caspase-1 in human Treg cells and the essential role of caspase-1 in the cleavage of IL-37 for FOXP3 expression suggest that an active inflammasome complex is present and integral to the functionality of human Treg cells. To determine which inflammasome signaling pathway contributes to caspase-1 activation in human Treg cells, an inflammasome-specific PCR array using human Treg cells and Tconv cells for comparison was preformed. Among differentially expressed genes (greater than 2-fold change and p-value <0.05) were 5 upregulated genes (CCL5, CCL7, HSP90AA1, HSP90AB1, and NAIP) and 4 downregulated genes (CXCL1, CXCL2, IL1B, and IL6) in human Treg cells compared to Tconv cells (FIGS. 25A-B). Both the intracellular inflammasome receptor neuronal apoptosis inhibitory protein (NAIP) and the chaperone protein, heat-shock protein 90 (HSP90), play a role in inflammasome assembly and activation (Mayor et. al., 2007; Zhao and Shao, 2015).

Elevated NAIP (FIG. 25C) and HSP90 (FIG. 25D) in human Treg cells compared to Tconv cells was confirmed. HSP90 binds to several Nod-like receptors (NLR), including NLRC4, NLRP2, NLRP3, NLRP4, NLRP12, and NOD1 (listed in Table 3).

TABLE 3
IPA: NLR family members that interact with HSP90

| HSP90 | Protein |
|---|---|
| HSP90 | NLRP12 |
| HSP90 | NLRP2 |
| HSP90 | NLRP3 |
| HSP90 | NLRP4 |
| HSP90 | NOD1 |
| HSP90B | NLRP12 |
| HSP90B | NOD1 |
| HSP90A | NLRC4 |
| HSP90A | NLRP12 |
| HSP90A | NLRP2 |
| HSP90A | NLRP3 |
| HSP90A | NLRP4 |
| HSP90A | NOD1 |

On the other hand, the ligand-bound NAIP receptor is known to form an active inflammasome complex solely with the Nod-like receptors family CARD domain containing 4 (NLRC4). As such, it was hypothesized that the elevated NAIP/NLRC4 inflammasome complex activates caspase-1, which is crucial for IL-37-induced FOXP3 expression in human Treg cells. Higher levels of NLRC4 (FIG. 25E) and its active form were found, pNLRC4 S533 (Qu et. al., 2012) (FIG. 25F), in Treg cells compared to Tconv cells. Whereas many NLR family members lack a caspase activation and recruitment domain (CARD) and necessitate an adaptor protein called apoptosis-associated speck-like protein containing a CARD (ASC) to recruit caspase-1 and form functional inflammasome (Franchi et. al., 2009), NLRC4 has a CARD that can directly recruit and activate caspase-1 without ASC (Mayor et. al., 2007; Vance, 2015). However, NLRC4 interaction with ASC further augments NLRC4 inflammasome formation and NLRC4-dependent caspase-1 activation (Broz and Dixit, 2016). Therefore, to determine the role of the NAIP/NLRC4 inflammasome complex in human Treg cells, ASC was silenced, NLRC4, and other inflammasome complexes in primary human Treg cells and measured caspase-1 activation using a FAM-FLICA® assay (FIG. 25G). The inhibition of ASC and NLRC4 led to a decrease in caspase-1 activation compared to the siRNA scramble control (Ctrl) (−20.5% and −41.5%, respectively) in Treg cells, whereas silencing NLRP2 and NLRP4 had minimal impacts. These findings suggest that NLRC4 is the NLR inflammasome complex responsible for caspase-1 activation. Additionally, NLRC4 was knocked down in purified CD4+CD25+CD127^{dim} Treg cells and purified CD4+ T cells to demonstrate that NLRC4 is necessary for FOXP3 gene (FIG. 25H) and protein (FIG. 25I) expression, respectively.

### Basal PAK1/2/3 Signaling Promotes NLRC4 Inflammasome Activation in Human Treg Cells

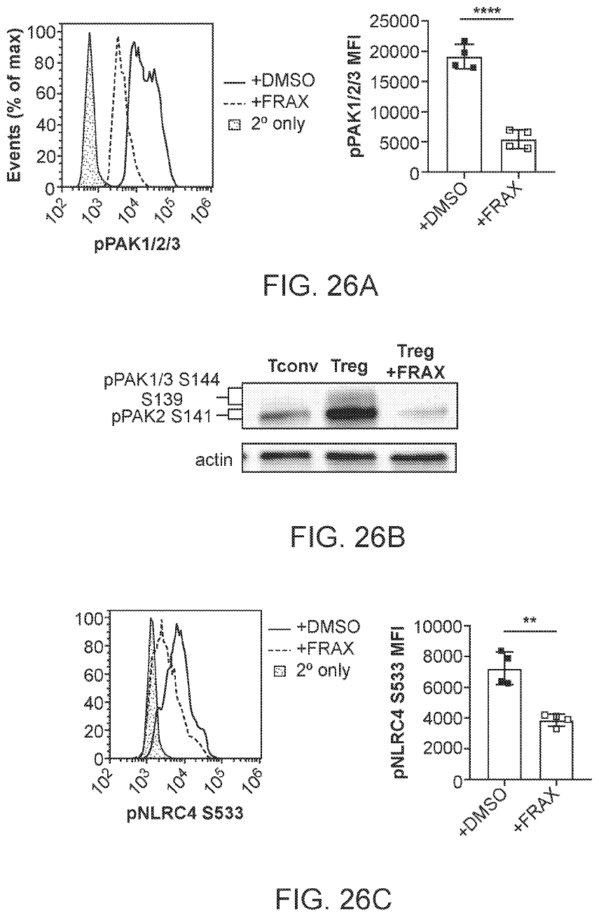

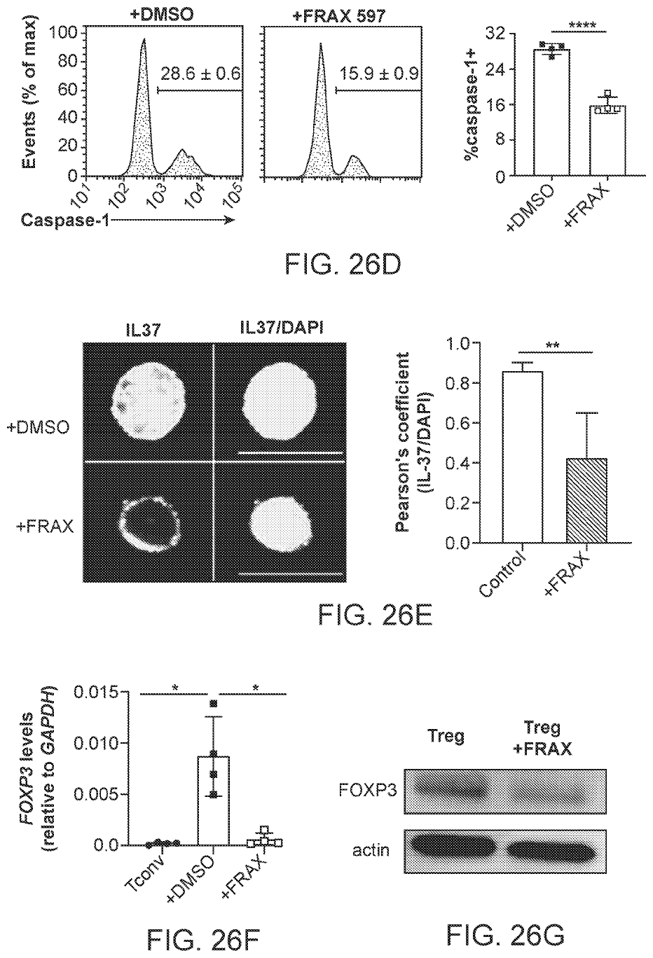

Next, the upstream signaling that could trigger NAIP/NLRC4 activation in Treg cells was examined. PKCS and the group 1 P21-activated kinases (PAKs) are crucial for NLRC4 phosphorylation (Qu et. al., 2012) and caspase-1 activation (Basak et. al., 2005). The PAKs, acting through high-affinity TCR signaling in the thymus during selection, are also necessary for FOXP3 expression and Treg stability (Choi et. al., 2018; O'Hagan et. al., 2015). Consequently, unstimulated human Treg cells were treated with the PAK1/2/3 inhibitor FRAX 597. It was observed that treatment with the inhibitor led to a 1.9-fold decrease in p-NLRC4 (S533) (confirmation of PAK1/2/3 inhibition in resting Treg cells by FRAX 597 is shown in FIGS. 26A-B, p-NLRC4 in FIG. 26C). In line with these findings, PAK1/2/3 inhibition also resulted in a notable reduction in caspase-1 activation (44% reduction, FIG. 26D), along with a shift in IL-37 localization from the nucleus to the cytoplasm (FIG. 26E), and a decrease in FOXP3 gene (FIG. 26F) and protein (FIG. 26G) expression. These results suggest that basal group 1 PAK activation is sufficient to activate NLRC4 inflammasome.

The data presented herein support the conclusion that PAK activation is required to activate NLRC4 inflammasome, which mediates caspase-1 activation and subsequent relocalization of IL-37 from the cytoplasm to the nucleus with p-SMAD3 S208 to induce FOXP3 expression in human Treg cells.

### Discussion

In this study it is revealed that human Treg cells FOXP3 expression requires the anti-inflammatory cytokine IL-37 via a signaling pathway that involves activation of caspase-1 by the NLRC4-inflammasome complex stimulated by PAK activation. Stability of peripheral FOXP3+ Treg cells has been well studied but has remained incomplete based on multiple reports showing the established signaling pathways required for Treg stability are dispensable (Hori, 2014; M. O. Li and Rudensky, 2016) (Schlenner et. al., 2012) ((Polansky et. al., 2008) (Chinen et. al., 2016; Rubtsov et. al., 2010). Stability of FOXP3 expression in human Treg cells is vital for supporting Treg suppressive function and in the maintenance of immune tolerance (Lu et. al., 2017).

Knockdown of IL37 or loss of the signaling pathway involved in the maturation of IL-37 in human Treg cells leads to a near loss of FOXP3+ Treg cells, showing the dependence of human Treg cells on IL-37. Inhibition of the IL-37 signaling pathway in WT mice, was also found to have no effect on Foxp3 expression (data not shown). A consistent finding between mouse and human Treg cells in our study was that elevated expression of IL-37 in Treg cells does lead to stability of FOXP3 expression in the presence of inflammatory conditions.

Immune tolerance, wherein DC-activated Treg cells maintain homeostatic conditions by preventing the generation of auto effector T cells, has been shown to involve IL-37 (Luo et. al., 2014). Treg cells provide multiple mechanisms to maintain tolerance, including IL-10 secretion, IL-2 consumption, and steric block of DCs (Sakaguchi et. al., 2008). Elevated levels of IL-37 in Treg cells suggest that IL-37 may be a novel mechanism for the development and function of tolerogenic Treg cells. IL-37 could have a homeostatic role rather than an immune suppressive role in Treg cells, and potentially play a role for the in vivo development of natural Treg cells.

The signaling required to induce FOXP3 expression and to maintain its expression has been well studied in mice. Mouse Treg cells are maintained through hypomethylated CpG islands in the promoter and in conserved non-coding DNA sequence (CNS or TSDR) of Foxp3 (Ohkura et. al., 2012; Yang et. al., 2015) through IL-2 signaling activation of transcription factors Stat5 that control Tet1 and Tet2 to support Foxp3 CNS hypomethylation (Ohkura et. al., 2012; Yang et. al., 2015). Peripheral IL-2, produced by autoreactive CD4+ Tconv cells, is required for immune tolerance by maintaining Treg cells (Setoguchi et. al., 2005; Stolley and Campbell, 2016). Conflicting studies have shown that loss of IL-2 in mice leads to only a slight decrease in Foxp3+ Treg cells, and the remaining Treg cells are functional at preventing autoimmunity (Chinen et. al., 2016) (Rubtsov et. al., 2010). While conflicting, these studies utilized mice to determine mechanisms of Treg cell maintenance. On the other hand, our study has examined human Treg cells due to the absence of IL-37 expression in mice (Nold et. al., 2010). This difference may suggest that the mechanism for FOXP3 expression is significantly different between mice and humans. Whereas some studies compared the difference between mouse and human Treg cells, the analyses were sometimes limited to Treg cell marker expression (Rodríguez-Perea et. al., 2016; Ziegler, 2006) or studied in the context of cancer biology (Akimova and Hancock, 2018), and molecular mechanisms have not been well compared or explored. In the current study, it was shown that IL37 knockdown or the loss of its upstream or downstream signaling involved in the maturation of IL-37 in human Treg cells led to a near complete loss of FOXP3+ Treg cells, showing the dependence of human Treg cells on IL-37.

Inflammatory IL-6 signaling can lead to the activation of DMNT1 in Treg cells resulting in methylation of the Foxp3 CNS and lower Foxp3 expression (Nair et. al., 2016; Yue et. al., 2016). IL37 Tg Treg cells maintained Foxp3 expression in the presence of the inflammatory cytokine, IL-6, in vitro and stabilized Treg cells in vivo during the hapten challenge by preventing their conversion to Tconv cells. Over the last decade, multiple functionally distinct Treg cell subsets have been reported and classified based in part on their heterogeneous expression of FOXP3 (Wing et. al., 2019). CD4+CD25+FOXP3^{hi} Treg cells are more suppressive and secrete high levels of IL-10 compared to CD4+CD25+FOXP3^{lo} Treg cells (Chauhan et. al., 2009; Miyara et. al., 2009; Strauss et. al., 2007; Wan and Flavell, 2007). In contrast, FOXP310 Treg cells convert to exTreg/effector T cells when activated with self-antigen, according to the “heterogeneity model” (Komatsu et. al., 2009) (Bailey-Bucktrout et. al., 2013). The instability and plasticity of Treg cells challenge the utilization of human Treg cell therapy to treat patients with autoimmunity. The data presented herein support the conclusion that in humans, FOXP3lo and FOXP3hi Treg cells have differential IL-37 expression. Treg cells expressing high levels of FOXP3 have high levels of IL-37; inversely, this is also true. IL37 Tg Treg cells are highly suppressive in vitro and in vivo compared to WT Treg cells and have sustained FOXP3 expression under inflammatory conditions. These findings that IL-37-expressing Treg cells are more likely to maintain the Treg cell functions during an inflammatory response may have significant implications for the treatment of autoimmunity as well as chronic inflammatory conditions.

It has been shown that immune tolerance, wherein IL-37-expressing DC-activated Treg cells maintain homeostatic conditions by preventing the generation of pathogenic T cells (Luo et. al., 2014). Treg cells provide multiple mechanisms to maintain tolerance, including IL-10 secretion, IL-2 consumption, and steric block of DCs (Sakaguchi et. al., 2008). Elevated levels of IL-37 in peripheral Treg cells suggest that IL-37 may be a novel mechanism for the development and function of tolerogenic Treg cells. IL-37 could have a homeostatic role rather than an immune suppressive role in Treg cells, potentially in the in vivo development of natural Treg cells. Thymic development of Treg cells has been shown to require TCR-dependent PAK signaling (Choi et. al., 2018) (O'Hagan et. al., 2015), and the data have demonstrated that PAK signaling is involved in NLRC4 and caspase-1 activation in Treg cells. These data suggest that during thymic Treg (tTreg) development, the IL-37 processing signaling pathways, including the NLRC4-inflammasome complex, may be involved. Further analyses of tTreg IL-37 expression and IL-37's role in Treg development are needed. Alternatively, as Treg cells enter the periphery and basal PAK signaling may dissipate with lower TCR engagement, different mechanisms, such as TLR5 signaling, can induce FOXP3 expression (Crellin et. al., 2005). In 2005, Crellin et. al. found that Treg cells have elevated expression of TLR5, and human Treg cells treated with flagellin (a ligand of TLR5) can induce FOXP3 (Crellin et. al., 2005). Where there is no clear report to link the TLR5 signaling pathway to FOXP3 expression, flagellin can directly activate NLRC4 through the receptor NAIP (Vance, 2015), which might lead to IL-37 processing and interaction with SMAD3 to induce FOXP3 expression in human Treg cells.

The inflammasome has recently been shown to play a role in the differentiation and development of Th1, Th2, and Th17 cells (Arbore et. al., 2016; Bruchard et. al., 2015; Martin et. al., 2016), but little has been explored on the role of inflammasomes in human Treg cells. Performing a PCR array of inflammasome components, it was shown that the chaperon protein HSP90 to be elevated in Treg cells compared to Tconv cells. HSP90, a known activator of the inflammasome (Mayor et. al., 2007), was determined to interact with the NLRC4-inflammasome complex and together activate caspase-1 in Treg cells. In the inflammasome PCR array, elevated expression of the cytosolic receptor NAIP which forms an activated inflammasome complex with NLRC4 was also found. Our data that ASC knockdown has significantly less impact on caspase-1 activation and FOXP3 expression compared to NLRC4 knockdown suggest that NLRC4 does not exclusively rely on the CARD domain provided by ASC but can use its own CARD domain to activate caspase-1. Overall, the distinct connections between IL-37 and Treg cell inflammasome activation, confirmed by multiple assays, shows strong support for a NLRC4 inflammasome-based mechanism required for FOXP3 expression in human Treg cells.

The mechanism for induction and continued expression of IL-37 in human Treg cells remains unknown. IL-37 expression in monocytes can be induced by TGF-β, suggesting that a similar signaling pathway is utilized for FOXP3 expression (Nold et. al., 2010). Previous studies have shown that TGF-β secreted from melanoma cell lines elevated IL-37 expression in human Treg cells. However, described herein is a mechanism by which resting (unstimulated) FOXP3+ human Treg cells are maintained in humans. IL-37 in Treg cells translocate to the nucleus via activated caspase-1 and induces not only an increase in FOXP3 expression but also stabilizes FOXP3 expression, allowing for sustained suppressive function in vitro and in vivo, even under inflammatory circumstances. These findings support a role for IL-37 as an essential cytokine in developing and maintaining human Treg cells and provide a novel role for the NLRC4 inflammasome and IL-37 in FOXP3 expression.

### Example 5: Control of Inflammatory and Immune Diseases In Vivo Using IL-37-Expressing Mouse Regulatory T Cells

The present Example herein demonstrates the effectiveness of IL-37-expressing mouse regulatory T cells (Treg cells) in controlling inflammatory and immune diseases in vivo, contrasting their performance with wild-type regulatory T cells lacking IL-37 expression. The results disclosed herein also provide a method for generating expanded IL-37-expressing mouse Treg cells for therapeutic use.

### Introduction

Immune tolerance is critical for suppressing autoimmunity, allergic reactions, and immune rejection. Dysregulation and loss of immune tolerance due to an inappropriate balance between proinflammatory or autoreactive T cells and Treg cells lead to the development of inflammatory diseases such as autoimmune disease, allergic reactions, chronic viral infection, and transplant rejection. Immunosuppressive drugs may be used to control these inflammatory conditions. However, they must be administered for a long time and often induces adverse effects such as systemic immunosuppression, leading to an increased risk of infections and malignancies. Treg cells are a specialized subpopulation of T cells that suppress and prevent inappropriate immune responses, thereby maintaining immune homeostasis and self-tolerance. Therefore, in recent years, researchers have used Treg cells to develop adoptive cellular therapies that can restore immune tolerance in autoimmune disease and transplantation. Because of its high target specificity, customization potential, and ability to generate immunological memory, Treg cell therapies have garnered significant attention among key industry stakeholders to treat various disease indications. However, whereas they have minimal side effects, clinical trials using an adoptive Treg infusion have shown poor persistency of Treg cells and expansion of undesired cytotoxic cells. While novel genome editing techniques, identification of stable Treg markers, and addition of Treg-supportive cytokines in vivo have improved the efficacy and expansion of primary Treg cells, translating the valuable findings into effective therapeutics has still proven challenging. These modified Treg cells may not be robust, persistent enough, or may not be antigen-specific.

Treg cells express the transcription factor, forkhead box P3 (FOXP3), to maintain their suppressive function. As FOXP3 loss results in dysfunctional Treg cells and therapeutic failure in autoimmunity and transplantation, the mechanisms that maintain FOXP3 mRNA expression in human Treg cells was studied and found that IL-37 transcriptionally regulates FOXP3 expression in human Treg cells, as shown in Examples 1 and 2. Dysregulated IL-37 expression has been reported in autoimmune diseases (RA, SLE, Hashimoto thyroiditis, inflammatory bowel disease, Graves' disease, multiple sclerosis), ankylosing spondylitis, asthma, cardiovascular diseases, cerebral ischemia, hepatic disorders, infections, type 2 diabetes, and cancers. Downregulated IL37 in active psoriasis skin was normalized when the JAK inhibitor tofacitinib controlled the disease. Similarly, downregulated IL37 in active atopic dermatitis skin was reversed after treatment with the JAK/SYK inhibitor, ASN002. These data clearly correlate IL37 expression with T-cell-mediated inflammation (Th17 in psoriasis and Th2 in atopic dermatitis), suggesting an immunosuppressive role for IL-37 in human diseases.

While 95 mammals have IL37 orthologs, this gene is not present in mice or rats. However, similar to other IL 1 family members, IL-37 has been shown to have no species specificity; thus, human-IL-37-expressing mouse cells show the functionality of IL-37. Indeed, Dinarello's group demonstrated that IL37 transgenic (Tg) mice exhibited a phenotype mimicking human IL-37 in human cells and inhibiting innate immunity in vitro and in vivo. In 2014, Fujita's group used the IL37 Tg mice to study contact hypersensitivity (CHS) induced by hapten 2,4-dinitrofluoro-benzene (DNFB) and reported that IL-37 promotes the generation of tolerogenic dendritic cells, thereby suppressing an antigen-specific immune response. This demonstrates IL-37's role in adaptive immunity and the maintenance of Foxp3+ Treg cells.

The results presented in Example 2 further demonstrated a crucial role for IL-37 in human Treg cells by sustaining FOXP3 expression and maintaining Treg cell stability, thereby regulating immune responses and fostering tolerance. These significant findings suggest a promising translational avenue for utilizing IL-37-expressing Treg cells as adoptive cell immunotherapy for treating autoimmune disorders and various inflammatory diseases. Since the adoptive cell therapy of Treg cells requires the isolation and in vitro expansion of Treg cells, in this Example 5, IL37 Tg mouse Treg cells were expanded and WT Treg cells (with no IL-37 expression). Their phenotypes and functions were compared in vitro and examined their therapeutic potential using various mouse models of immune and inflammatory conditions, including antigen-specific contact hypersensitivity model (CHS), Th-17-mediated psoriasis model, and neuroinflammation model such as traumatic brain injury (TBI). All diseases are very prevalent, affecting about 10% (CHS), 2-4% (psoriasis), and 40% (TBI) of populations. As Treg cells suppress various immune responses and provide tolerance, they can be used to treat various autoimmune diseases (such as Crohn's disease, systemic lupus erythematosus) and transplant recipients (solid organ transplantation or hematopoietic stem cell transfer), as well as diabetes mellitus, graft versus host disease (GVHD), bipolar disorder, Alzheimer's disease, allergic rhinoconjunctivitis, and COVID19. Some groups have also targeted neurodegenerative diseases and other chronic inflammation-related diseases, such as cardiovascular and metabolic diseases. Some data using type-1 diabetes (a type of autoimmune disease) and GVHD in Example 6 (but not in this Example 5) was included. Altogether, developing modified Treg cells that are robust, persistent, and effective in vivo would have a significant human impact.

### Results

### Ex Vivo Expanded IL37 Mouse Treg Cells Demonstrate Superior Proliferative Capacity, Higher Foxp3 Expression, Enhanced Suppressive Function In Vitro, and do not Express Il17 Compared to Expanded WT Mouse Treg Cells.

Treg cells constitute only ˜10% of CD4+ T cells in the human body, posing a significant challenge in purifying an effective dosage of Treg cells for adoptive immunotherapy. To address this challenge, various labs employ a strategy to expand the Treg cell population following purification using a cocktail of anti-CD3/CD28 and a high dose of IL-2. However, another obstacle that arises after cell expansion and adoptive transfer is the loss of expanded Treg cells upon adoptive transfer. Therefore, generating an adequate number of Treg cells with sustained suppressive function in vivo is crucial. Example 2 showed that IL37 mouse Treg cells displayed sustained and more robust suppressive function than WT mouse Treg cells; therefore, both types of Treg cells were expanded and proliferation, function, and phenotypes was analyzed.

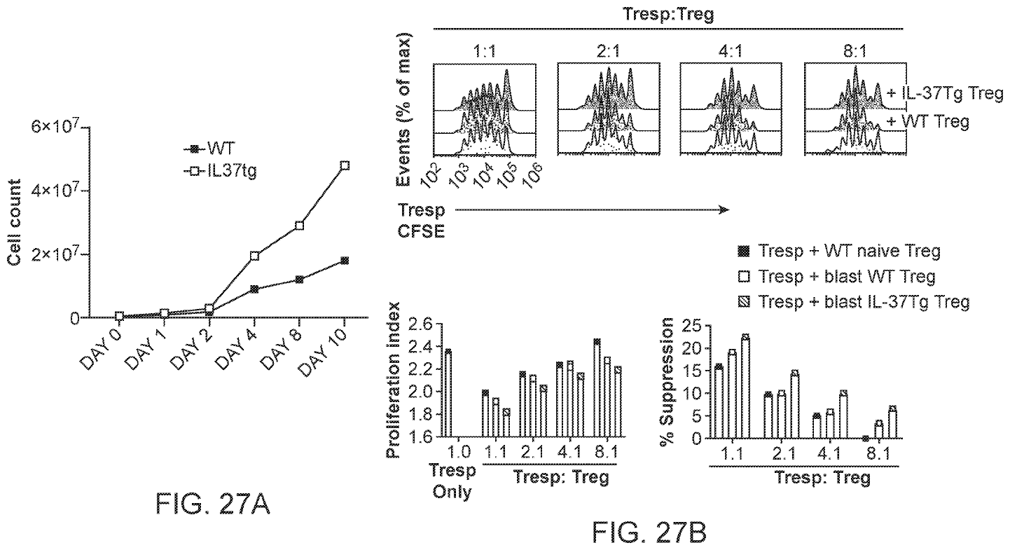

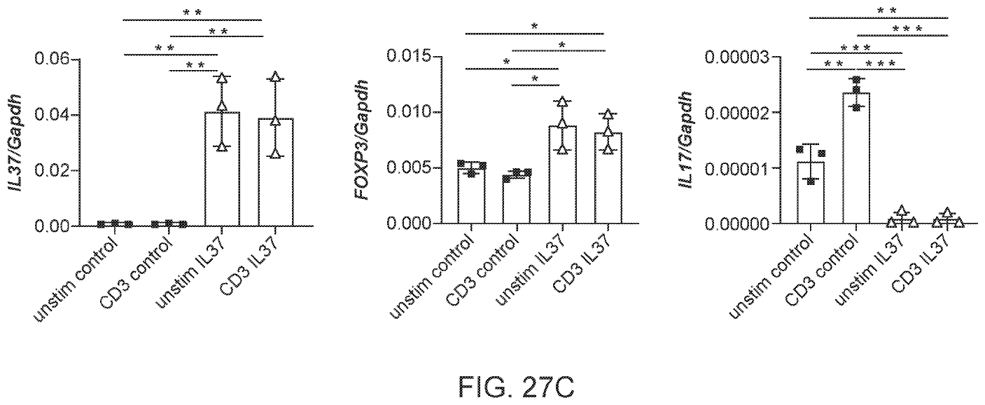

IL37 Treg cells exhibited accelerated proliferation compared to WT Treg cells (FIG. 27A). Compared to non-expanded WT Treg cells, both WT and IL37 Treg cells were more suppressive in vitro following expansion. However, expanded IL37 Treg cells demonstrated superior suppression to inhibit Tresp cell (naïve CD4+CD25− T responder) proliferation compared to expanded WT Treg cells (FIG. 27B). TCR stimulation in expanded Treg cells did not affect IL37 expression in IL37 Treg cells (FIG. 27C). Consistent with these findings, Foxp3 expression remained highly elevated in expanded IL37 Treg cells compared to expanded WT Treg cells. Notably, expanded WT Treg cells exhibited increased expression of Il17, a Th17 cell marker and effector cytokine, whereas its expression remained low in expanded IL37 Treg cells. These results suggest that the expansion process may render Treg cells susceptible to Th17 lineage conversion, whereas the presence of IL-37 protects Treg cells from undergoing this shift.

### Ex Vivo Expanded IL37 Mouse Treg Cells but not Expanded WT Treg Cells Remain Highly Suppressive In Vivo to Control Contact Hypersensitivity.

The suppressive activity of expanded IL37 mouse Treg cells was further analyzed in vivo using a contact-hypersensitivity (CHS) model where hapten (dinitrofluorobenzene, DNFB)-sensitized WT mice were i.v. injected with expanded WT or IL37 Treg cells and subsequently challenged with DNFB (FIG. 28A). This model was employed in Example 4 utilizing non-expanded Treg cells (FIG. 8). Consistent with the previous findings presented in FIG. 8, expanded IL37 Treg cells effectively suppressed CHS responses upon adoptive transfer (showing ˜80% reduction at 48 h). However, this reduction was not observed in mice receiving expanded WT Treg cells (FIGS. 28B-C).

Furthermore, tracking of CFSE-labeled cells revealed the presence of adoptively transferred Treg cells in the DNFB-challenged ear tissue specimens in both types of mice (FIG. 28D). However, CFSE+ conventional T cells (Tconv) were also detected in the ears of mice that received expanded WT Treg cells, whereas they were absent in mice that received expanded IL37Tg Treg cells. These findings demonstrate that, despite the expansion, expanded WT Treg cells remained unstable and incapable of suppressing immune responses in vivo. In contrast, expanded IL37 Treg cells exhibited a sustained and highly suppressive capacity in vivo.

### Expanded IL37 Mouse Treg Cells are Highly Suppressive to Inhibit Established Inflammation in a Mouse Model of Psoriasis

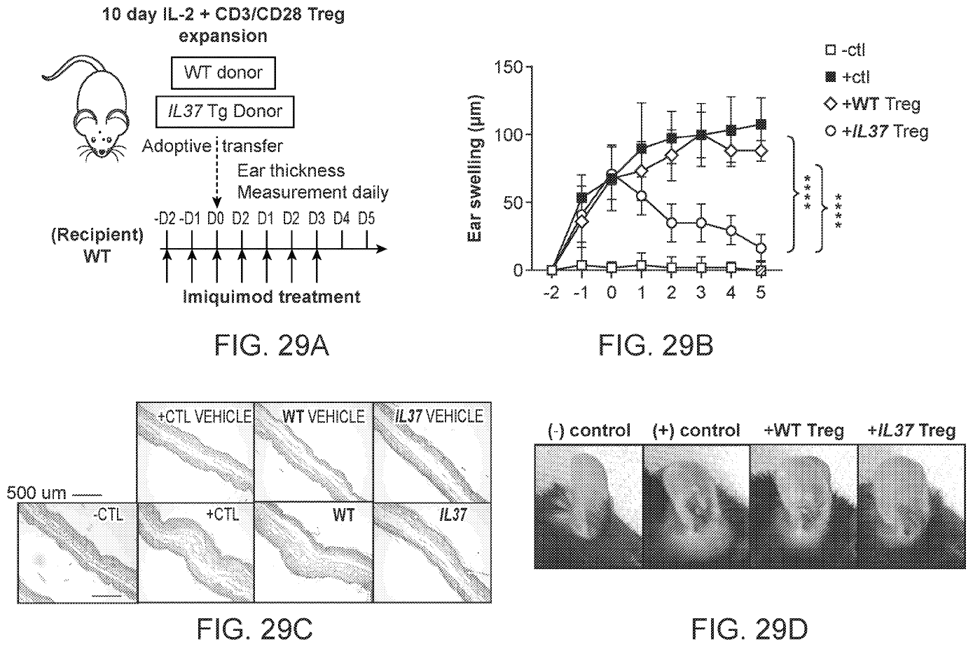

Next, a mouse model of psoriasis was employed to evaluate the suppressive capabilities of expanded IL37 Treg cells in an inflammatory context. Imiquimod (Aldara), a potent immune activator and a ligand for TLR7 and TLR8, is known to exacerbate psoriasis lesions in patients and induce psoriasis-like skin changes in mice. Therefore, imiquimod has been utilized in studying psoriasis pathogenesis and therapeutics in mice. In this model, mice were subjected to daily application of 5% imiquimod cream on the right ear for 7 consecutive days, with a daily dose of 3.125 mg of the active compound (FIG. 29A, schematic of psoriasis model). This dosage was determined empirically to induce optimal and reproducible skin inflammation in mice.

The psoriatic responses (erythema, scaling, and thickness) were scored daily on a scale from 0 to 4. On day 0 (48 hours after the initial imiquimod application), mice received adoptive transfer of either CFSE+ expanded WT or IL37 Treg cells. The effects of expanded IL37 Treg cells on controlling ear swelling were observed starting 24 hours after the Treg cell adoptive transfer (three days after the initial imiquimod treatment) (FIG. 29B). Due to the daily application of imiquimod cream, inflammation worsened each day in mice that received no treatment or expanded WT Treg cells. In contrast, mice that received expanded IL37 Treg cells exhibited diminishing ear swelling every day, leading to nearly normal-appearing skin by day 5 (indicating an approximately 80% reduction of ear swelling compared to control skin). This was confirmed by histological analysis (FIG. 29C) and clinical evaluation (FIG. 29D). Importantly, cell counts of the ears, spleen, and cervical lymph nodes supported significant lymphoproliferation in (+) control and expanded WT Treg mice compared to (−) control and expanded IL37 Treg mice (data not shown).

These findings unequivocally demonstrate that expanded IL37 Treg cells possess a high suppressive capacity to inhibit established inflammation in vivo effectively. In contrast, WT Treg cells do not exhibit the same level of effectiveness.

### Expanded IL37 Mouse Treg Cells Effectively Reduce Neurological Severity Scores and T-Cell-Mediated Inflammation Induced by Traumatic Brain Injury (TBI) in a TBI Mouse Model

Traumatic brain injury (TBI) is a significant public health problem; its lifetime prevalence is up to 40% among adults. The most common causes of TBI are falls and motor vehicle accidents, and TBI is the leading cause of death in children over one year old. After the primary injury, secondary injury gradually progresses from minutes to years. Among the various pathways contributing to secondary injury in TBI, immune activation and inflammation play a crucial role in determining clinical and functional outcomes. However, traditional approaches targeting immune activation have not improved clinical outcomes in TBI patients. A potential strategy to switch the immune balance toward the regulatory arm is the utilization of adoptive Treg cell therapy.

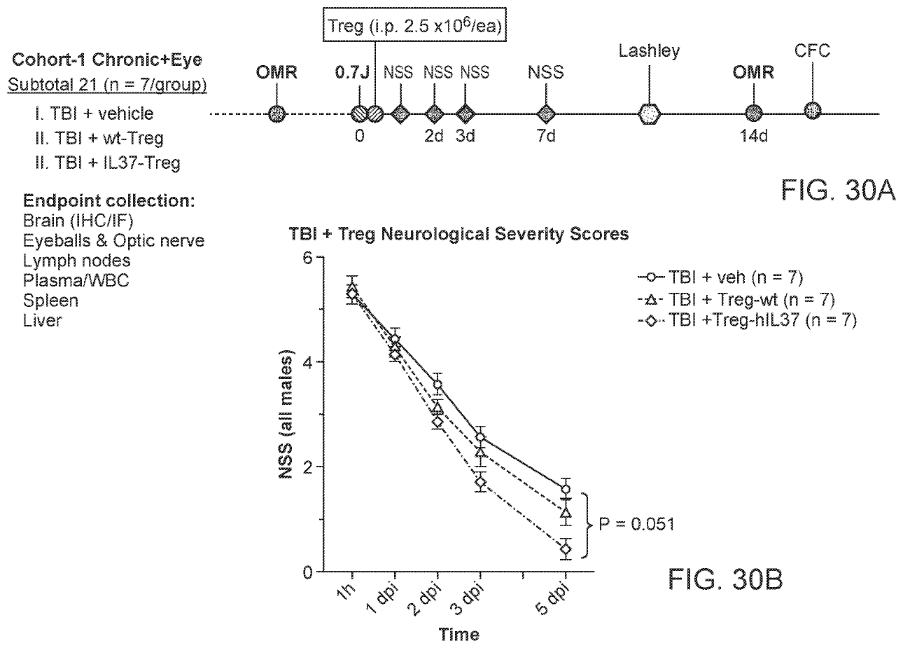

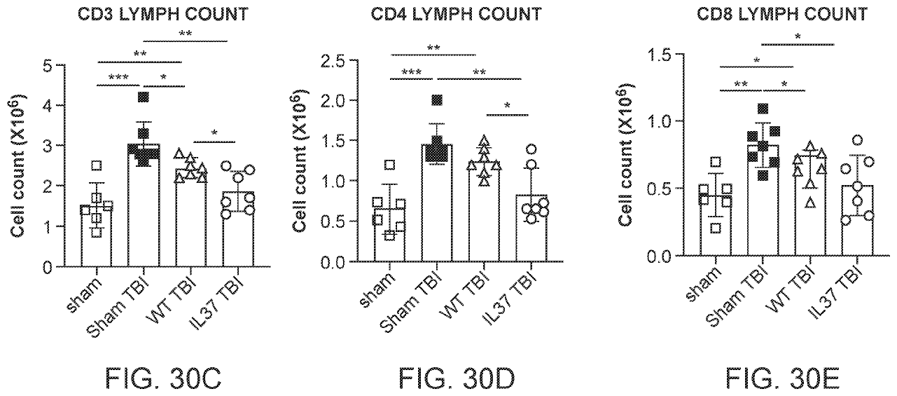

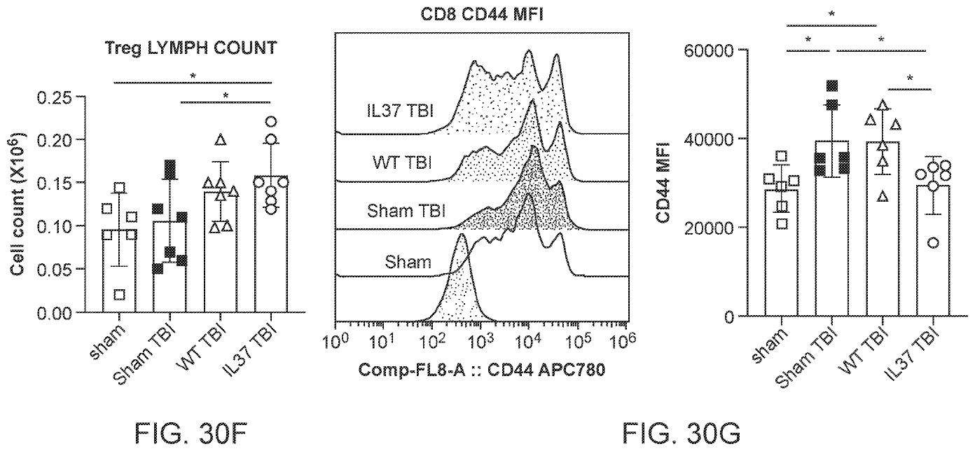

To investigate the potential of IL-37 in reducing inflammation induced by TBI, a TBI model (0.7J×1) was employed in 4-month-old C57BL/6 mice. 3 hours after inducing TBI, expanded WT or IL37 Treg cells were adoptively transferred into WT mice (refer to the scheme of our TBI experiment in FIG. 30A). The results demonstrated significantly lower neurological severity scores in mice treated with IL37 Treg cells compared to mice injected with PBS or WT Treg cells (p=0.014 on day 2 and p=0.0002 on day 5) (FIG. 30B). Surprisingly, by day 5, the scores were nearly diminished to zero in mice treated with IL-37-expressing Treg cells.

Further analysis of the cervical lymph nodes revealed significantly lower counts of CD3+, CD4+, and CD8+ T cells in mice treated with expanded IL37 Treg cells compared to those with no treatment (sham TBI) or treated with expanded WT Treg cells (WT) (FIGS. 30C-E). Notably, mice that received IL37 Treg cells exhibited CD3+/CD4+/CD8+ T cell counts similar to those without TBI or sham, suggesting the successful suppression of T cell-mediated immune responses by IL37 Treg cells following TBI induction. Additionally, in FIG. 30F, a significant increase in Treg cell number in the cervical lymph nodes of mice that received IL37 Treg cells was observed, compared to those that received vehicle control.

Moreover, it was found that the expression of the activation marker CD44 on CD8+ T cells in the cervical lymph nodes of TBI mice treated with IL37 Treg cell was significantly lower compared to TBI mice with no treatment (sham TBI) or WT Treg cells (WT) (FIG. 30G), suggesting that IL37 Treg cells effectively reduced the activation of cytotoxic CD8+ T cells, potentially preventing TBI-mediated secondary inflammation.

### Discussion:

In this Example, the data illustrate a novel approach to mitigate immune responses and inflammation in mouse models of contact hypersensitivity, psoriasis, and traumatic brain injury (TBI) by employing adoptive transfer of expanded IL-37-expressing mouse Treg cells. Treg cells harvested and expanded from genetically modified IL-37-expressing mice demonstrate enhanced proliferation and maintain elevated Foxp3 expression, rendering them more functionally suppressive than expanded WT mouse Treg cells both in vitro and in vivo.

### Conclusion:

This Example demonstrates the therapeutic potential of IL-37-expressing mouse Treg cells in managing inflammatory and immune diseases. These findings highlight the significance of IL-37 as a critical regulator in modulating immune responses and offer a promising avenue for developing novel treatments targeting these diseases.

### Example 6: Mitigation of Xenogeneic Graft-Versus-Host Disease Using IL-37-Expressing Human Regulatory T Cells

The present Example herein elucidates the distinct differences between the ability of expanded human Treg cells and those overexpressing IL-37 to regulate inflammation and immune responses in vivo. Moreover, it exhibits the therapeutic promise of IL-37-expressing human Treg cells in mitigating various human inflammatory and immune disorders, including graft-versus-host disease (GVHD). The results disclosed herein also outline a method for generating stable Treg cells for therapeutic applications.

### Introduction

Human inflammatory and immune disorders often arise or persist due to the dysfunction of Treg cells. Adoptive cellular transfer (ACT) therapy involving expanded human Treg cells has gained prominence in the past decade as a means to address inflammatory and immune disorders and conditions associated with Treg cellular dysfunction. However, a challenge in ACT is that the human Treg cells requiring expansion are the patient's Treg cells with dysfunctional and unstable features, and expansion or genetic engineering alone may not rectify Treg cell functionality. By utilizing IL-37 overexpression, which regulates FOXP3 and promotes Treg cell stability, the challenges related to human Treg cell instability and dysfunction in ACT can be overcome, as demonstrated in Examples 1 and 2.

To conduct a preclinical study of human Treg cells overexpressing IL-37, a specific mouse model capable of accommodating human cells was required. However, the xenogeneic response provoked by mouse immune cells towards human cells poses a challenge in investigating human T cells or Treg cells in vivo. Nevertheless, mouse models of xenogeneic graft-versus-host disease (GVHD), triggered by transferring human peripheral blood mononuclear cells (PBMC) into immunocompromised NOD-scid IL-2Ra^{-/-} (NSG) mice or BALB/c Rag2^{-/-}Il2rg^{-/-}SirpaNOD (BRGS) mice, offer invaluable tools for studying human immune function in vivo. As these mice lack functional T, B, and NK cells, they enable efficient engraftment of human T cells. These mice can be subsequently utilized to investigate the effects of human Treg cells on human PBMC-induced GVHD.

By employing adoptive transfer of IL-37-expressing human Treg cells in a mouse model of GVHD, it was demonstrated that IL-37 enhances Treg stability and significantly mitigates GVHD responses compared to control human Treg cells. These results highlight the therapeutic potential of IL-37 in enhancing Treg cell functionality in vivo and underscore their therapeutic promise in reducing inflammation and immune responses across various human disorders and conditions.

### Results

### Type 1 Diabetes (T1D) Patients Exhibit Reduced Treg Cell Counts and Diminished IL37 Expression in Treg Cells.

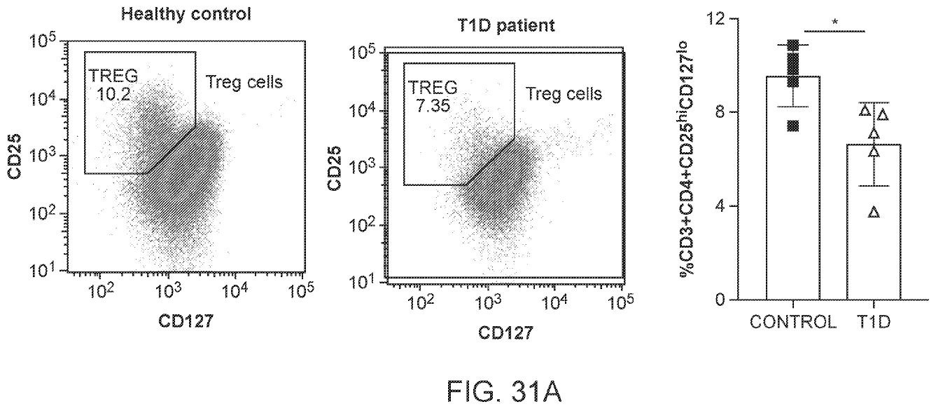

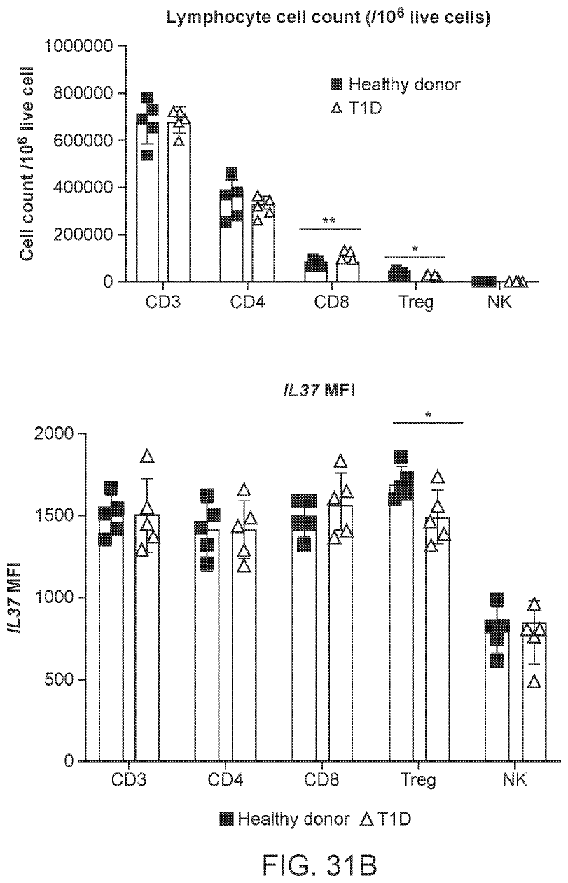

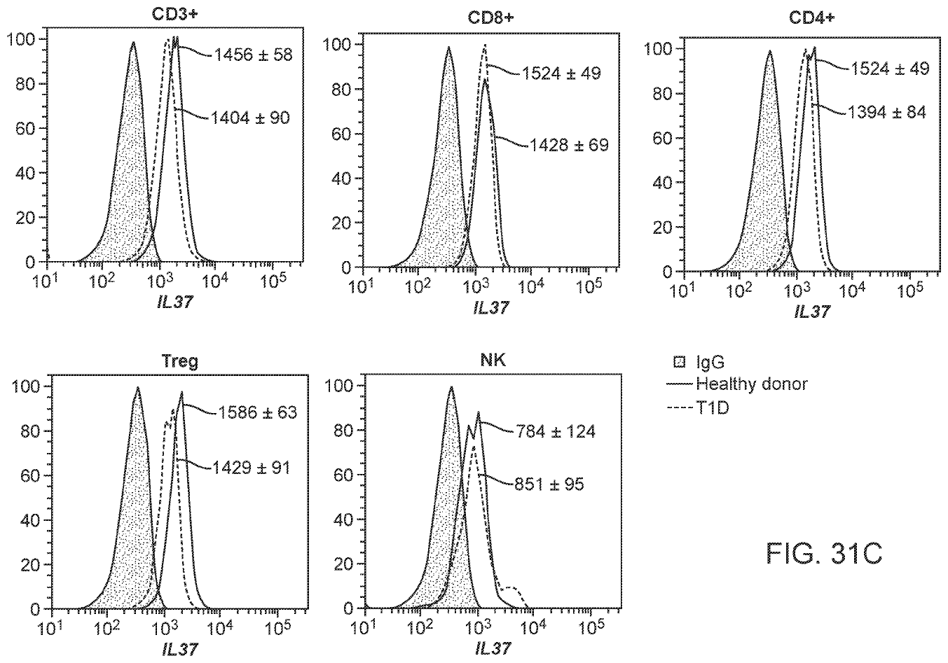

Evidence supports that type 1 diabetes (T1D) is caused by an immune-mediated attack of pancreatic beta cells and that an inappropriate balance between autoreactive T cells and regulatory T (Treg) cells impairs peripheral tolerance. As described in Example 2, FIG. 21, FOXP3 expression is regulated by 3 signaling pathways such as TCR, IL-2, and TGF-β, which activate TFs (NFAT, STAT5, and SMAD3) to FOXP3 transcription. It is well known that Treg cells from T1D patients have decreased suppressive function and impaired IL-2 receptor (IL-2R) signaling. Aberrant IL-2R signaling decreases STAT5 phosphorylation and contributes to decreased FOXP3 expression in Treg cells in TID patients. In FIGS. 31A-B, it was confirmed that TID patients have significantly lower percentages (A) and amount (B) of Treg cells in the peripheral blood compared to healthy donors.

Considering the unique role of IL-37 in human Treg cells, it is also possible that some TID patients may have aberrant IL-37 expression, further contributing to low FOXP3 expression in Treg cells. The Primeflow® assay (described in FIG. 23) was used to assess the expression of IL37 in multiple human immune cells in TID patients (FIG. 31C). Only Treg cells from TID showed a significant reduction in IL37 expression, suggesting that reduced IL-37 expression may lead to Treg cell instability and their conversion into non-Treg CD4+ T cells, with a subsequent decrease in Treg cell number and function. Therefore, improving the Treg cell function ex vivo and in situ using adoptive cell transfer of autologous Treg cells expressing IL-37 could present a compelling therapeutic strategy for TID patients.

### Ex Vivo Expanded Human Treg Cells Exhibit Diminished IL37 Expression, Reduced FOXP3 Levels, and Increased Th17 Gene Expression

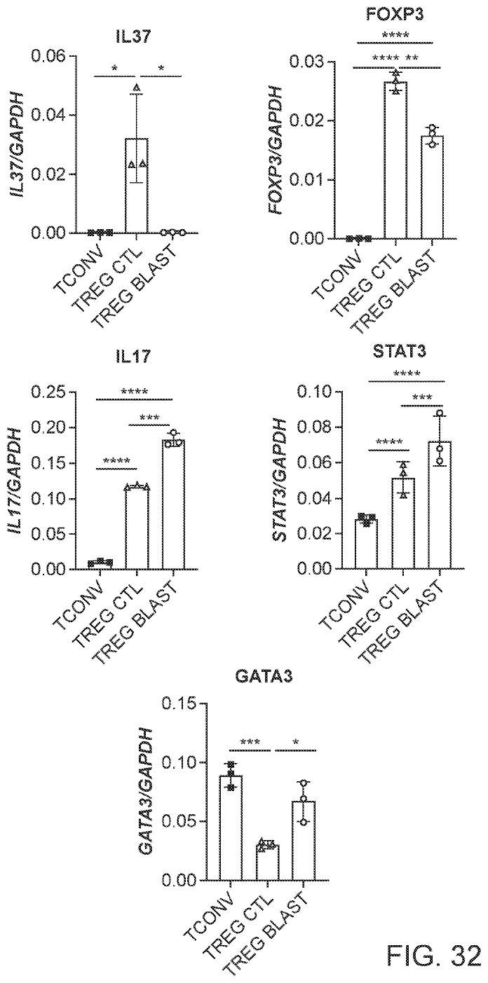

Because the therapeutic use of adoptive transfer of Treg cells often requires the ex vivo expansion of Treg cells, a method for expanding and transducing primary human Treg cells was developed. Building on a protocol described by Jeff Bluestone's lab and refined by other groups, purified human Treg cells were expanded. After a 10-day expansion period, the gene expression of these expanded Treg cells was analyzed and compared to human Tconv and naïve Treg cells (prior to expansion) (FIG. 32). Naïve Treg cells demonstrated elevated IL-37 levels compared to Tconv cells, which aligns with our findings in Example 4. However, expanded (blast) human Treg cells exhibited diminished IL37 expression and lower FOXP3 levels than naïve Treg cells. They also displayed increased gene expression of Tconv leaneage, such as IL17, STAT3, and GATA3. These elevated Th17 markers, along with decreased FOXP3 levels, suggest that these expanded human Treg cells may become susceptible to convertion into a more proinflammatory phenotype.

### IL37 Overexpression Enhances the Stability of Expanded Human Treg Cells.

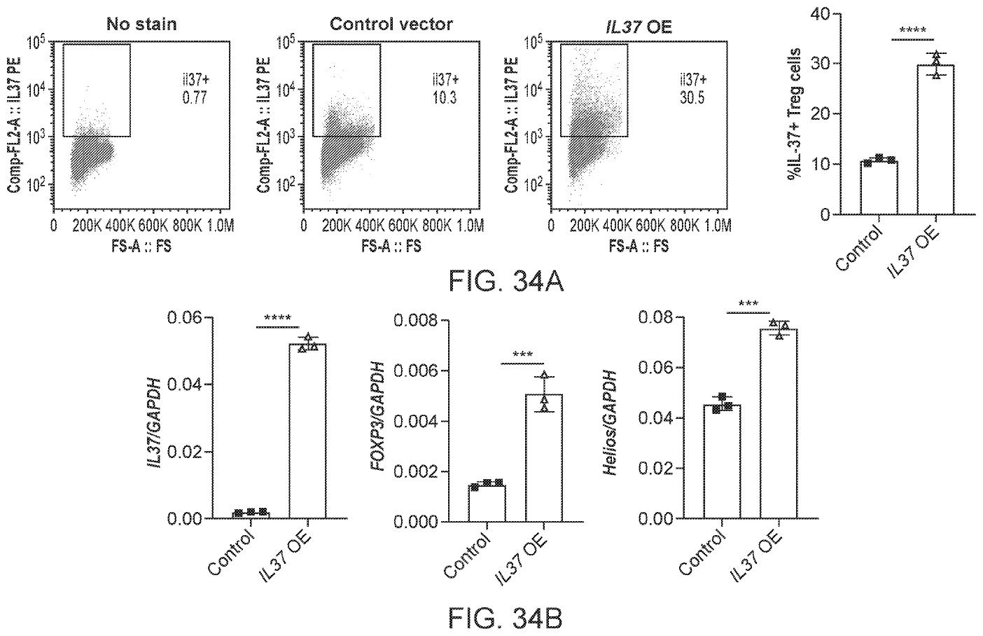

Given that expanded human Treg cells did not express IL37 mRNA, they were transduced with a control or IL37 overexpressing (OE) lentivirus vector (FIG. 33). Flow cytometry density plots confirmed the overexpression of IL-37 in IL37OE vector-transduced expanded human Treg cells as shown in FIG. 34A, which was further confirmed by qRT-PCR in FIG. 34B. Gene expression analysis in FIG. 34B also demonstrated a significant increase in FOXP3 and Helios, the marker for Treg cell stability, compared to expanded human Treg cells transduced with the empty control vector. These findings suggest that IL37 overexpression enhances the stability of expanded human Treg cells.

Ex Vivo Expanded Human Treg Cells Fail to Suppress Xenogeneic GVHD, but Overexpression Sustains their Suppressive Function In Vivo

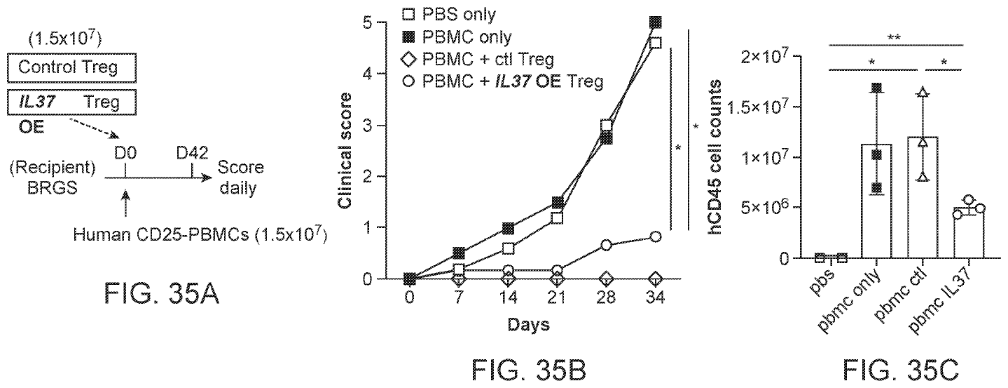

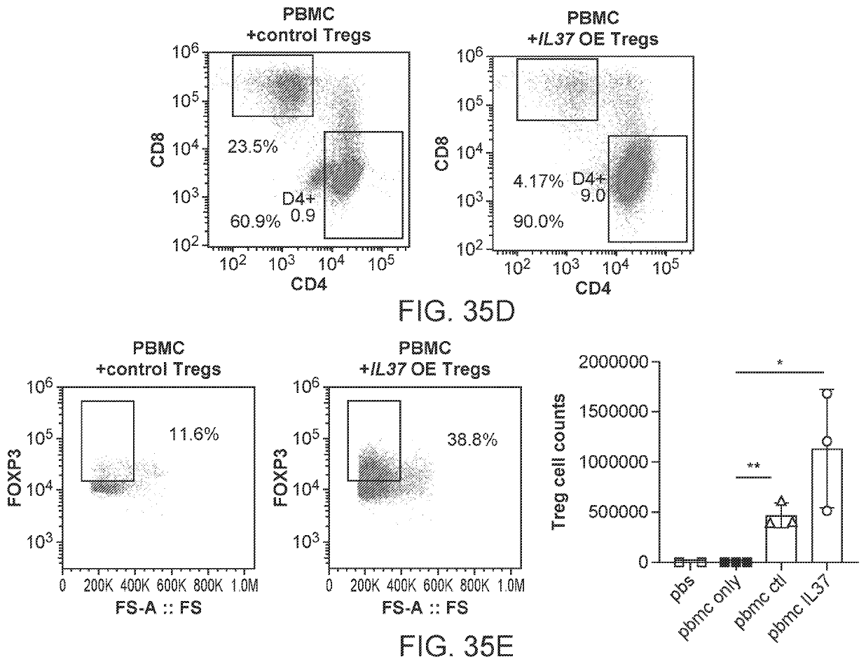

To investigate the suppressive function of IL37-overexpressing expanded human Treg cells in vivo, a mouse model of xenogeneic GVHD (xenoGVHD) was employed, as described in the Introduction. This model involves the adoptive transfer of human PBMCs into immunocompromised BALB/c Rag2^{-/-}Il2rg^{-/-}Sirpa^{NOD} (BRGS) mice, which lack lymphocytes (T, B, and NK cells) and SIRPA, enabling efficient engraftment of human PBMC and subsequent xenogeneic reactions. Human PBMCs, after depletion of CD4+CD25+CD127lo T (Treg) cells, were adoptively transferred into BRGS mice to induce GVHD. Mice were then co-transferred with PBS, control-vector-transduced expanded human Treg cells, or IL37-overexpressing (OE) expanded human Treg cells, as per the schematic in FIG. 35A. Utilizing the scoring criteria from our methods section (Example 1), it was noted that mice receiving IL37 OE Treg cells exhibited minimal clinical scores until day 21. Whereas they began showing some signs of GVHD later, their clinical scores remained lower than those of mice that received either PBMC only or PBMC+control Treg cells, even 34 days post-PBMC injection (FIG. 35B). The mice that received PBMC only or PBMC+control Treg cells displayed splenomegaly and severe clinical findings (ruffled fur, hair loss, skin scaling, and hunching), but mice treated with IL37OE Treg cells presented only mild to moderate changes, affirming the capacity of IL-37-overexpressing expanded primary human Treg cells to suppress GVHD in mice.

Splenocyte analysis revealed that mice receiving IL37OE Treg cells had significantly fewer human CD45+ (FIG. 35C), CD3+, and CD4+ (FIG. 35D) cells than mice treated with control Treg cells. Although both groups of mice exhibited detectable amounts of human CD4+ T cells, most CD4+ T cells in mice receiving IL37OE Treg cells were identified as Treg cells (FIG. 35E).

These findings suggest that expanded human Treg cells are insufficient in suppressing GVHD, whereas IL-37 overexpression in these cells preserves their suppressive function in vivo. This is likely attributable to the stabilization of FOXP3 expression and enhanced Treg cell stability under inflammatory conditions. Taken in conjunction with the mouse Treg data described in Example 5, these results underscore the therapeutic potential of IL-37-expressing Treg cells in mitigating inflammation and immune responses across various human disorders and conditions.

### Discussion

Example 6 illustrates a stark contrast in the capacity of expanded human Treg cells and those overexpressing IL-37 to modulate inflammation and immune responses in vivo, utilizing a xenogeneic mouse model of GVHD. The mice receiving expanded human Treg cells with IL-37 overexpression exhibited significantly lower clinical scores, reduced human lymphocyte proliferation, and maintained elevated levels of human Treg cells even after one month. These findings emphasize the pivotal role of IL37 in enabling expanded human Treg cells to overcome instability and dysfunction under inflammatory and immune conditions by bolstering Treg cell stability and enhancing suppressive function. Overall, this Example supports the utility of IL37 overexpression in the adoptive cell therapy of human Treg cells.

### EMBODIMENTS

Embodiment 1. A method of producing a population of modified CD4+ T cells comprising introducing into a plurality of human T cells, a composition comprising an Interleukin-37 (IL-37) or a nucleic acid sequence encoding the IL-37 under suitable conditions that express the IL-37 in the nucleus of the human T cell, thereby producing a plurality of modified CD4+ T cells.

Embodiment 2. The method of embodiment 1, wherein the population of modified CD4+ T cells are regulatory T cells.

Embodiment 3. The method of embodiment 2, wherein the population of modified CD4+ T cells are non-regulatory T cells.

Embodiment 4. The method of any one of embodiments 1-3, wherein the nuclear expression of IL-37 in the plurality of modified CD4+ T cells is at least about 5-fold greater than the nuclear expression of IL-37 in a population of wildtype human T cells.

Embodiment 5. The method of any one of embodiments 1-3, wherein the nuclear expression of IL-37 in the plurality of modified CD4+ T cells is about 5-fold to about 10-fold greater than the nuclear expression of IL-37 in a population of wildtype human T cells.

Embodiment 6. The method of any one of embodiments 1-5, wherein at least about 75% of the plurality of modified CD4+ T cells express at least one marker of a regulatory T cell.

Embodiment 7. The method of embodiment 6, wherein at least about 95% of the plurality of modified CD4+ T cells express at least one marker of a regulatory T cell.

Embodiment 8. The method of any one of embodiments 6-7, wherein at least one marker of a regulatory T cell is selected from a group consisting of FOXP3, CD25, CD4, CTLA4, IL-10, GITR, TGF-beta and CD127.

Embodiment 9. The method of embodiment 8, wherein at least one marker is FOXP3.

Embodiment 10. The method of embodiment 8, wherein at least one marker is FOXP3 and CD25.

Embodiment 11. A composition comprising a population of modified CD4+ T cells produced by the method of any one of embodiments 1-10.

Embodiment 12. The composition of embodiment 11, for use in the treatment of a immune disease or disorder selected from the group consisting of: allergic contact hypersensitivity, graft versus host disease, transplant rejection, type 1 diabetes, systemic lupus erythematosus, inflammatory bowel disease, Crohn's disease, ulcerative colitis and multiple sclerosis.

Embodiment 13. The composition of embodiment 12, wherein the immune disease or disorder is allergic contact hypersensitivity.

Embodiment 14. The composition of embodiment 12, wherein the immune disease or disorder is graft versus host disease.

Embodiment 15. The composition of embodiment 12, wherein the immune disease or disorder is inflammatory bowel disease.

Embodiment 16. A method of treating an immune disease or disorder comprising administering to a subject in need thereof a therapeutically effective amount of a composition comprising a population of modified CD4+ T cells that express nuclear IL-37.

Embodiment 17. The method of embodiment 16, wherein the population of modified CD4+ T cells are regulatory T cells.

Embodiment 18. The method of any one of embodiments 16-17, wherein the expression of nuclear IL-37 in the population of modified CD4+ T cells is at least about 50% greater than the expression of nuclear IL-37 in a population of wildtype CD4+ T cells.

Embodiment 19. The method of embodiment 18, wherein the expression of nuclear IL-37 in the population of modified CD4+ T cells is about 50% to about 80% greater than the expression of nuclear IL-37 in a population of wildtype CD4+ T cells.

Embodiment 20. The method of any one of embodiments 16-19, wherein the population of modified CD4+ T cells are allogeneic CD4+ T cells.

Embodiment 21. The method of any one of embodiments 16-19, wherein the population of modified CD4+ T cells are autologous CD4+ T cells.

Embodiment 22. The method of embodiment any one of embodiments 16-21, wherein the immune disease or disorder is selected from the group consisting of: allergic contact hypersensitivity, graft versus host disease, transplant rejection, type 1 diabetes, systemic lupus erythematosus, inflammatory bowel disease, Crohn's disease, ulcerative colitis and multiple sclerosis.

Embodiment 23. The method of embodiment 22, wherein the immune disease or disorder is allergic contact hypersensitivity.

Embodiment 24. The method of embodiment 22, wherein the immune disease or disorder is graft versus host disease.

Embodiment 25. The method of embodiment 22, wherein the immune disease or disorder is type 1 diabetes.

1. A method of producing a population of modified CD4⁺ T cells comprising introducing into a plurality of human T cells, a composition comprising an Interleukin-37 (IL-37) or a nucleic acid sequence encoding the IL-37 under suitable conditions that express the IL-37 in the nucleus of the human T cell, thereby producing a plurality of modified CD4⁺ T cells.

2. The method of claim 1, wherein the population of modified CD4⁺ T cells are regulatory T cells.

3. The method of claim 2, wherein the population of modified CD4⁺ T cells are non-regulatory T cells.

4. The method of claim 1, wherein the nuclear expression of IL-37 in the plurality of modified CD4⁺ T cells is at least 5-fold greater than the nuclear expression of IL-37 in a population of wildtype human T cells.

5. The method of claim 1, wherein the nuclear expression of IL-37 in the plurality of modified CD4⁺ T cells is about 5-fold to about 10-fold greater than the nuclear expression of IL-37 in a population of wildtype human T cells.

6. The method of claim 1, wherein at least about 75% of the plurality of modified CD4⁺ T cells express at least one marker of a regulatory T cell.

7. The method of claim 6, wherein at least about 95% of the plurality of modified CD4⁺ T cells express at least one marker of a regulatory T cell.

8. The method of claim 6, wherein said at least one marker of a regulatory T cell is selected from a group consisting of FOXP3, CD25, CD4, CTLA4, IL-10, GITR, TGF-beta and CD127.

9. The method of claim 8, wherein said at least one marker is FOXP3.

10. The method of claim 8, wherein said at least one marker is FOXP3 and CD25.

11. A composition comprising a population of modified CD4⁺ T cells produced by a method comprising introducing into a plurality of human T cells, a composition comprising an Interleukin-37 (IL-37) or a nucleic acid sequence encoding the IL-37 under suitable conditions that express the IL-37 in the nucleus of the human T cell.

12-16. (canceled)

17. A method of treating an immune disease or disorder or an inflammatory disease or disorder comprising administering to a subject in need thereof a therapeutically effective amount of a composition comprising a population of modified CD4⁺ T cells that express nuclear IL-37.

18. The method of claim 17, wherein the population of modified CD4⁺ T cells are regulatory T cells.

19. The method of claim 17, wherein the expression of nuclear IL-37 in the population of modified CD4⁺ T cells is at least 50% greater than the expression of nuclear IL-37 in a population of wildtype CD4⁺ T cells.

20. The method of claim 19, wherein the expression of nuclear IL-37 in the population of modified CD4⁺ T cells is about 50% to about 80% greater than the expression of nuclear IL-37 in a population of wildtype CD4⁺ T cells.

21. The method of claim 17, wherein the population of modified CD4⁺ T cells are allogeneic CD4⁺ T cells.

22. The method of claim 17, wherein the population of modified CD4⁺ T cells are autologous CD4⁺ T cells.

23. The method of claim 17, wherein the immune disease or disorder is selected from the group consisting of: allergic contact hypersensitivity, graft versus host disease, transplant rejection, type 1 diabetes, systemic lupus erythematosus, inflammatory bowel disease, Crohn's disease, ulcerative colitis and multiple sclerosis.

24. The method of claim 23, wherein the immune disease or disorder is allergic contact hypersensitivity.

25. The method of claim 23, wherein the immune disease or disorder is graft versus host disease.

26. The method of claim 23, wherein the immune disease or disorder is type 1 diabetes.

27. The method of claim 17, wherein the inflammatory disease or disorder is selected from the group consisting of psoriasis, traumatic brain injury, bronchitis and pneumonitis.
